SEQUENCE LISTING (280 sequences)

SEQ ID NO: 1 - DNA, 20 bp, Artificial Sequence
  cagApro probe
gttgataacgctgtcgcttc

SEQ ID NO: 2 - DNA, 20 bp, Artificial Sequence
  P1S1 vacA-derived probe
ggagcrttrgtcagcatcac

SEQ ID NO: 3 - DNA, 21 bp, Artificial Sequence
  P22S1a vacA-derived probe
gctttagtaggagcrttrgtc

SEQ ID NO: 4 - DNA, 20 bp, Artificial Sequence
  P1S1b vacA-derived probe
ggagcgttgattagykccat

SEQ ID NO: 5 - DNA, 19 bp, Artificial Sequence
  P2S1b vacA-derived probe
gttttagcaggagcgttga

SEQ ID NO: 6 - DNA, 20 bp, Artificial Sequence
  P1S2(VAS2) vacA-derived probe
gctaayacgccaaaygatcc

SEQ ID NO: 7 - DNA, 20 bp, Artificial Sequence
  P2S2 vacA-derived probe
gatcccatacacagcgagag

SEQ ID NO: 8 - DNA, 19 bp, Artificial Sequence
  P1M1 vacA-derived probe
ttgatacgggtaatggtgg

SEQ ID NO: 9 - DNA, 20 bp, Artificial Sequence
  P2M1 vacA-derived probe
gggtaatggtggtttcaaca

SEQ ID NO: 10 - DNA, 21 bp, Artificial Sequence
  P1M2 vacA-derived probe
acgaatttaagagtgaatggc

SEQ ID NO: 11 - DNA, 21 bp, Artificial Sequence
  P2M2 vacA-derived probe
agagcgataacgggctaaaca

SEQ ID NO: 12 - DNA, 24 bp, Artificial Sequence
  cagF primer
ttgaccaacaaccacaaaccgaag

SEQ ID NO: 13 - DNA, 22 bp, Artificial Sequence
  cagR primer
cttcccttaattgcgagattcc

SEQ ID NO: 14 - DNA, 20 bp, Artificial Sequence
  VA1XR primer
cctgaraccgttcctacagc

SEQ ID NO: 15 - DNA, 20 bp, Artificial Sequence
  M1F primer
gtggatgcycatacrgctwa

SEQ ID NO: 16 - DNA, 20 bp, Artificial Sequence
  M1R primer
rtgagcttgttgatattgac

SEQ ID NO: 17 - DNA, 21 bp, Artificial Sequence
  HPMGF primer
cacagccactttcaataacga

SEQ ID NO: 18 - DNA, 20 bp, Artificial Sequence
  HPMGR primer
cgtcaaaataattccaaggg

SEQ ID NO: 19 - DNA, 19 bp, Artificial Sequence
  cagSF primer
caacaaccacaaaccgaag

SEQ ID NO: 20 - DNA, 21 bp, Artificial Sequence
  cagSR primer
gattggtttttgatcaggatc

SEQ ID NO: 21 - DNA, 20 bp, Artificial Sequence
  cagFN1 primer
gataagaaygatagggataa

SEQ ID NO: 22 - DNA, 18 bp, Artificial Sequence
  cagRN1 primer
aatactgattctttttgg

SEQ ID NO: 23 - DNA, 20 bp, Artificial Sequence
  VAMSFb primer
gtggatgcccatacggctaa

SEQ ID NO: 24 - DNA, 20 bp, Artificial Sequence
  VAMSFc primer
gtggatgctcatacagctwa

SEQ ID NO: 25 - DNA, 20 bp, Artificial Sequence
  VAMSFd primer
gtggatgcccatacgatcaa

SEQ ID NO: 26 - DNA, 20 bp, Artificial Sequence
  VAMSFe primer
gcgagcgctcatacggtcaa

SEQ ID NO: 27 - DNA, 20 bp, Artificial Sequence
  cagprobe3 cagA-derived probe
ggatttttgatcgctttatt

SEQ ID NO: 28 - DNA, 20 bp, Artificial Sequence
  P3S1 vacA-derived probe
gggytattggtyagcatcac

SEQ ID NO: 29 - DNA, 20 bp, Artificial Sequence
  P4S1 vacA-derived probe
gctttagtrgggytattggt

SEQ ID NO: 30 - DNA, 19 bp, Artificial Sequence
  P1M1new vacA-derived probe
ttgatackggtaatggtgg

SEQ ID NO: 31 - DNA, 20 bp, Artificial Sequence
  P2M1new vacA-derived probe
kggtaatggtggtttcaaca

SEQ ID NO: 32 - DNA, 20 bp, Artificial Sequence
  P1M2new vacA-derived probe
kggtaatggtggtttcaaca

SEQ ID NO: 33 - DNA, 21 bp, Artificial Sequence
  P2M2new vacA-derived probe
agagcgataayggkctaaaca

SEQ ID NO: 34 - DNA, 21 bp, Artificial Sequence
  P1M3 vacA-derived probe
agggtagaaatggtatcgaca

SEQ ID NO: 35 - DNA, 23 bp, Artificial Sequence
  HpdiaS1 vacA-derived probe
catgcygccttctttacaaccgt

SEQ ID NO: 36 - DNA, 23 bp, Artificial Sequence
  HpdiaS2 vacA-derived probe
catgccgcctttttcacraccgt

SEQ ID NO: 37 - DNA, 23 bp, Artificial Sequence
  HpdiaS3 vacA-derived probe
catgccgctctttttacaaccgt

SEQ ID NO: 38 - DNA, 23 bp, Artificial Sequence
  HpdiaS4 vacA-derived probe
catgccgccttttttacaaccgt

SEQ ID NO: 39 - DNA, 23 bp, Artificial Sequence
  HpdiaS5 vacA-derived probe
agtcgcgcytttttyacaaccgt

SEQ ID NO: 40 - DNA, 184 bp, Artificial Sequence
  Helicobacter pylori vacA nucleic acid sequence
ccctctggtttctctcgctttagtaggagcattggtcagcatcacaccgcaacaaagtcatgccgcctttttcacaaccgtgatcattccagccattgttgggggtatcgctacaggcaccgctgtaggaacggtctcagggcttcttagttggggactaaaacaagccgaagaagccaataaa

SEQ ID NO: 41 - DNA, 199 bp, Artificial Sequence
  Helicobacter pylori vacA nucleic acid sequence
tcgccctctngtttctctcgctttagtagnagcattngtcagcatcacaccgcaacanagtcatgccgcctttttcacaaccgtnatcattccagccattgttgggggtatngctacaggcaccgctgtaggaacggtctcagggcttcttagttggggactaaaacaagccgaagaagccaataaaaccccagataaa

SEQ ID NO: 42 - DNA, 227 bp, Artificial Sequence
  Helicobacter pylori vacA nucleic acid sequence
gaatcgccctytggtttctcttgctttagtaggagcattgrttagyrycayaccgcaacaaagtcatgccgcctttttyacraccgtgatcattccagccattgttggrggtatcgctacaggcactgctgtaggaacggtctcagggcttcttagttggggrctcaaacaagccgaagaagcsaataaaaccccrgataaacccgataaagtttggcgcattcaag

SEQ ID NO: 43 - DNA, 176 bp, Artificial Sequence
  Helicobacter pylori vacA nucleic acid sequence
gccctttagtttctctcgctttagtggggttattggtcagcatcacaccacaaaaaagtcatgccgcctttttcacaaccgtgatcattccagccattgttggaggtatcgctacaggtgctgctgtaggaacggtctcagggcttcttggttgggggctcaaacaagccgaagaa

SEQ ID NO: 44 - DNA, 185 bp, Artificial Sequence
  Helicobacter pylori vacA nucleic acid sequence
gccctctggtttctctcgctttagtaggagcattggtcagcatcacaccgcaacaaagtcatgccgcctttttcacaaccgtgatcattccagccattgttggaggtatcgctacaggcgctgctgtaggaacggtctcagggcttcttagctgggggctcaaacaagccgaagaagccaataaa

SEQ ID NO: 45 - DNA, 204 bp, Artificial Sequence
  Helicobacter pylori vacA nucleic acid sequence
aatcgccctctggtttctctcgctttagtaggagcattggtcagcatcacaccgcaacaaagtcatgccgccttttttacaaccgtgatcattccagccattgttggaggtatcgctacaggcgctgctgtaggaacggtctcagggcttcttagctgggggctcaaacaagccgaacaagccaataaagccccggacaaaccc

SEQ ID NO: 46 - DNA, 207 bp, Artificial Sequence
  Helicobacter pylori vacA nucleic acid sequence
gaatcgccctttagtttctcttgctttagtaggagcattggtcagcatcacaccgcaacaaagtcatgccgcctttttcacaaccgtgatcattccagccattgttgggggtatcgctacaggcgctgctgtaggaacggtttcagggcttcttggctgggggctaaaacaagccgaagaagccaataaaaccccagataaacccga

SEQ ID NO: 47 - DNA, 207 bp, Artificial Sequence
  Helicobacter pylori vacA nucleic acid sequence
caatcgccctctagtttctctcgctttagtaggagcattggtcagcatcacaccgcaacaaagtcatgccgcctttttcacaaccgtgatcattccagccattgtggggggtatcgctacaggcgctgctgtaggaacggtctcagggcttcttagctgggggctcaaacaagccgaagaagccaataaaaccccggacaaacccga

SEQ ID NO: 48 - DNA, 207 bp, Artificial Sequence
  Helicobacter pylori vacA nucleic acid sequence
caatcgccctctggtttctcttgctttagtaggagcgttagtcagcatcacaccgcaacaaagtcatgccgcctttttcacaaccgtgatcattccagccattgttggggggatcgctacaggcgctgctgtaggaacggtctcagggcttcttagctgggggctcaaacaagccgaagaagccaataaaaccccagataaacccga

SEQ ID NO: 49 - DNA, 176 bp, Artificial Sequence
  Helicobacter pylori vacA nucleic acid sequence
ccctttagtttctcttgttttagcaggagcgttgattagcgccataccgcaacaaagtcatgccgcctttttcacgaccgtgatcattccagccattgttgggggtatcgctacaggcaccgctgtaggaacggtttcagggcttcttagctgggggctcaaacaagccgaagaag

SEQ ID NO: 50 - DNA, 187 bp, Artificial Sequence
  Helicobacter pylori vacA nucleic acid sequence
ccctttagtttctcctgttttagcaggagcgttgattagctccataccgcaacaaagtcatgccgcctttttcacaaccgtgatcattccagccattgttgggggtatcgctacaggcactgctgtaggaacggtctcagggcttcttagctgggggctcaaacaagcygaasaagcsaataaagcc

SEQ ID NO: 51 - DNA, 193 bp, Artificial Sequence
  Helicobacter pylori vacA nucleic acid sequence
cttgagtttctcttgttttagcaggagcgttgattagcgccataccgcaacaaagtcatgccgcctttttcacgaccgtgatcattccagccattgttgggggtatcgctacaggcaccgctgtaggaacggtttcagggcttcttagctgggggctcaaacaagccgaagaagccaataaaaccccagataa

SEQ ID NO: 52 - DNA, 196 bp, Artificial Sequence
  Helicobacter pylori vacA nucleic acid sequence
gccctttagtttctctcgttttagcaggagcgttgattagctccataccgcaacaaagtcatgccgcctttttcacaaccgtgatcattccagccattgttgggggtatcgctacaggcaccgctgtaggaacggtttcagggcttcttagctgggggctcaaacaagccgaacaagccaataaagccccggacaa

SEQ ID NO: 53 - DNA, 131 bp, Artificial Sequence
  Helicobacter pylori vacA nucleic acid sequence
aatcgccctttagtttctcttgttttagcaggagcgttgattagcgccataccgcaacaaagtcatgccgcctttttcacgaccgtnatcattccagccattgttnnnngtatcgctacaggcaccgctgt

SEQ ID NO: 54 - DNA, 185 bp, Artificial Sequence
  Helicobacter pylori vacA nucleic acid sequence
ctacgccctttagtttctctcgttttagcaggagcgttgattagcgccataccgcaacaaagtcatgccgcctttttcacaaccgtgatcattccagccattgttgggggcatcgcttcaggcgctgctgtaggaacggtctcagggcttcttagttggggactcaaacaagccgaagaagcgaa

SEQ ID NO: 55 - DNA, 201 bp, Artificial Sequence
  Helicobacter pylori vacA nucleic acid sequence
aatcgccctttagtttctctcgttttagcaggagcgttgattagcgccataccgcaagagagtcatgccgcctttttcacaaccgtnatcattccagccattgttgggggtatcgctacaggcaccgctgtaggaacggtctnagggcttyttagttggggactnwaacaagccgaagaagccaataaaaccccggataaa

SEQ ID NO: 56 - DNA, 187 bp, Artificial Sequence
  Helicobacter pylori vacA nucleic acid sequence
ccctttagtttctctcgttttagcannagcgttgattagcrccataccgcaagagagtcatgccgccttttttacaaccgtnatcattccagccattgttgggggtatcgctacaggygctgctgtaggaacggtctcagggcttcttagctgggggctcaaacaagccgaacaagccaataaagcc

SEQ ID NO: 57 - DNA, 187 bp, Artificial Sequence
  Helicobacter pylori vacA nucleic acid sequence
ccctttggtttctctcgttttagcaggagcgttgattagctccataccgcaagagagtcatgccgcctttttcacaaccgtgatcattccagccattgttgggggtatcgctacaggcgctgctgtaggaacggtctcagggcttcttagctgggggctcaaacaagccgaagaagcgaataaaacc

SEQ ID NO: 58 - DNA, 187 bp, Artificial Sequence
  Helicobacter pylori vacA nucleic acid sequence
ccctttagtttctctcgttttagcaggagcgttgattagctccataccgcaagagagtcatgccgcctttttcacaaccgtgatcattccagccattgttgggggtatcgctacaggcaccgctgtaggaacggtctcagggcttcttagctggggactcaaacaagccgaagaagcgaataaaacc

SEQ ID NO: 59 - DNA, 185 bp, Artificial Sequence
  Helicobacter pylori vacA nucleic acid sequence
ccctttagtttctctcgttttagcagnagcgttnattagtgccataccgcaananagtcatgccgcctttttcacnaccgtnatcattccagccattgttgggggtatcgccacaggcaccgctgtaggaacggtctcagggcttcttagttggggactnaaacaagccgaagaagcgaataaaa

SEQ ID NO: 60 - DNA, 199 bp, Artificial Sequence
  Helicobacter pylori vacA nucleic acid sequence
caggagcgttgattagtgccataccgcaagagagtcatgccgcctttttcacgaccgtgatcattccagccattgttgggggtatcgccacaggcaccgctgtaggaacggtctcagggcttcttagttggggactcaaacaagccgaagaagcgaataaaaccccagtataaacccgataaagtttggcgcattcaag

SEQ ID NO: 61 - DNA, 188 bp, Artificial Sequence
  Helicobacter pylori vacA nucleic acid sequence
tttctctcgttttagcaggagcgttgattagctccataccgcaagagagtcatgccgcctttttcacaaccgtgatcattccagccattgttgggggtatcgctacaggcgctgctgtaggaacggtctcagggcttcttagctgggggctcaaacaagccgaagaagcgaataaaaccccagataaa

SEQ ID NO: 62 - DNA, 206 bp, Artificial Sequence
  Helicobacter pylori vacA nucleic acid sequence
gaatcgccctttagtttctctcgttttagcaggagcgttgattagtgccataccgcaagagagtcatgccgcctttttcacgaccgtgatcattccagccattgttgggggtatcgccacaggcaccgctgtaggaacggtctcagggcttcttagttggggactcaaacaagccgaagaagcgaataaaaccccagtataaaccc

SEQ ID NO: 63 - DNA, 206 bp, Artificial Sequence
  Helicobacter pylori vacA nucleic acid sequence
aatcgccctttggtttctctcgttttagcaggagcgttgattagctccataccgcaagagagtcatgccgcctttttcacaaccgtgmtcattccagccattgttgggggtatggctacaggcgctgctgtaggaacggtctnagggcttyttagctgggggctnwaacaagccgaagaagcgaataaaaccccagataaacccga

SEQ ID NO: 64 - DNA, 201 bp, Artificial Sequence
  Helicobacter pylori vacA nucleic acid sequence
aatcgccctattatttctctcgttttagcaggagcgttgattagctccataccgcaagaaagtcatgccgcctttttcacgaccgtgatcattccagccattgttgggggtatcgccacaggcgctgctgtaggaacggtctcagggcttcttagctgggggctcaaacaagccgaacaagccaataaagccccggacaaa

SEQ ID NO: 65 - DNA, 197 bp, Artificial Sequence
  Helicobacter pylori vacA nucleic acid sequence
gccctttagtttctcttgttttagcaggagcgttgattagtgccataccgcaagagagctatgccgcctttttcacaaccgtgatcattccagccattgttgggggtatcgctacaggcaccgctgtaggaacggtctcagggcttcttagttggggactcaaacaagccgaagaagcgaataaaaccccagataaa

SEQ ID NO: 66 - DNA, 201 bp, Artificial Sequence
  Helicobacter pylori vacA nucleic acid sequence
aatcgccctttagtttctctcgttttagcaggagcgtttattagcgccataccgcaagagagtcatgccgcctttttcacgaccgtgatcattccagccattgttgggggtatcgccacaggcaccgctgtaggaacggtctcagggcttcttagttggggactcaaacaagccgaagaagcgaataaaaccccagataaa

SEQ ID NO: 67 - DNA, 195 bp, Artificial Sequence
  Helicobacter pylori vacA nucleic acid sequence
gtgggggtgttaatgggcaccgaactaggggctaatacgccaaacgatcccatacacagcgagagtcgcgcctttttcacaaccgtgatcattccagccattgttgggggtatcgccacaggcactgctgtaggaacggtctcagggcttcttagttggggactcaaacaagccgaagaagcgaataaaacccca

SEQ ID NO: 68 - DNA, 196 bp, Artificial Sequence
  Helicobacter pylori vacA nucleic acid sequence
agtgggggtgttaatgggcactgaactaggggctaacacgccaaacgatcccatacacagcgagagtcgcgccttttttacaaccgtgatcattccagccattgttgggggtatcgctacaggcgctgctgtaggaacggtttcagggcttcttagctgggggctcaaacaagccgaacaagccaataaagccccg

SEQ ID NO: 69 - DNA, 196 bp, Artificial Sequence
  Helicobacter pylori vacA nucleic acid sequence
tagtgggggcattaatgagtaccgaactaggggctaacacgccaaatgatcccatacacagcgagagtcgcgccttttttacaaccgtgatcattccagccattgttgggggtatcgctacaggcgctgctgtaggaacggtctcagggcttcttagtcgggggctcaaacaagccgaacaagccaataaagcccc

SEQ ID NO: 70 - DNA, 232 bp, Artificial Sequence
  Helicobacter pylori vacA nucleic acid sequence
caatcgccctattatctctctcgctctagtgggggtgttaatgggtaccgaactaggggctaacacgccaaacgatcccatacacagcgagagtcgcgccttttttacaaccgtgatcattccagccattgttgggggtatcgctacaggcgctgctgtaggaacggtttcagggcttcttagctgggggctcaaacaagccgaacaagccaataaagccccggacaaaccc

SEQ ID NO: 71 - DNA, 228 bp, Artificial Sequence
  Helicobacter pylori vacA nucleic acid sequence
aatcgccctattatctctctcgctctagtgggggtgttaatgggtaccgaactaggggctaacacgccaaacgatcccatacacagcgagagtcgcgcctttttcacaaccgtgatcattccagccattgttggaggtatcgctacaggtgctgctgtaggaacggtctcagggcttcttagctgggggctcaaacaagccgaacaagccaataaagccccggacaaa

SEQ ID NO: 72 - DNA, 228 bp, Artificial Sequence
  Helicobacter pylori vacA nucleic acid sequence
aatcgccctattatctctctcgctttagtgggggtrttaatgggcaccgaactaggggctaacacgccaaacgatcccatacacagcgagagtcgcgcctttttcacaaccgtgatcattccagccattgttgggggtatcgctacaggcgctgctgtaggaacggtctcagggcttcttagctgggggctcaaacaagccgaacaagccaataaagccccggataaa

SEQ ID NO: 73 - DNA, 233 bp, Artificial Sequence
  Helicobacter pylori vacA nucleic acid sequence
aatcgccctattatctctctcgctttagagggggtgttaataggcaccgaactaggggctaacacgccaaatgatcccatacacagcgagagtcgcgccttttttacaaccgttattattccagccattgttgggggtatcgctacaggcgctgctgtaggaacggtctcagggcttcttagctgggggctcaaacaagccgaacaagccaataaagccccggataaacccga

SEQ ID NO: 74 - DNA, 300 bp, Artificial Sequence
  Helicobacter pylori vacA nucleic acid sequence
tttaaaggtggatgctcatacagctaattttaaaggtattgatacgggtaatggtggtttcaacaccttagattttagtggtgttacaggtaaggtcaatatcaacaagctcatcacagcttccactaatgtggccgttaaaaacttcaacattaatgaattgattgttaaaaccaatggtgtgagtgtgggggaatacactcattttagcgaagatataggcagtcaatcgcgcatcaataccgtgcgtttggaaactggcactaggtcaatcttttctgggggtgttaaatttaaagg

SEQ ID NO: 75 - DNA, 300 bp, Artificial Sequence
  Helicobacter pylori vacA nucleic acid sequence
tttaaaggtggatgctcatacagctaattttaaaggtattgatacgggtaatggtggtttcaacaccttagattttagtggtgttacaggtaaggtcaatatcaacaagctcattacggcttccactaatgtggccgttaaaaacttcaacattaatgaattgttggttaagaccaatggggtgagtgtgggggaatacactcattttagcgaagatataggcagtcaatcgcgcatcaataccgtgcgtttggaaactggcactaggtcaatcttttctgggggtgtcaaatttaaagg

SEQ ID NO: 76 - DNA, 300 bp, Artificial Sequence
  Helicobacter pylori vacA nucleic acid sequence
tttaaaagtggatgctcatacagctaattttaaaggtattgatacgggtaatggtggtttcaacaccttggattttagtggcgttacagacaaagtcaatatcaacaagctcatcacagcttccactaatgtggccattaaaaacttcaacattaatgaattgttggttaagaccaatggggtgagtgtgggggaatacactcattttagcgaagatataggcagtcaatcgcgcatcaacaccgtgcgtttagaaactggcactaggtcaatcttttctgggggtgtcaaatttaaaag

SEQ ID NO: 77 - DNA, 300 bp, Artificial Sequence
  Helicobacter pylori vacA nucleic acid sequence
tttaaaggtggatgctcatacagctaattttaaaggtattgatacgggtaatggtggtttcaacaccttagattttagtggtgttacaggtaaggtcaatatcaacaagctcatcacagcttccactaatgtggccgctaaaaacttcaacattaatgaattgattgttaaaaccaatggggtgagtgtgggggaatacactcattttagcgaagatataggcagtcaatcgcgcatcaataccgtgcgtttggaaactggcactaggtcaatctattctggcggtgttaaatttaaagg

SEQ ID NO: 78 - DNA, 300 bp, Artificial Sequence
  Helicobacter pylori vacA nucleic acid sequence
tttaaaagtggatgctcatacagctaattttaaaggtattgatacgggtaatggtggtttcaacaccttagattttagtggtgttacaggtaaggtcaatatcaacaagctcatcacagcttccactaatgtggccgctaaaaacttcaacattaatgaattgattgttaaaaccaatggggtgagtgtgggggaatacactcattttagcgaagatataggcagtcaatcgcgcatcaataccgtgcgtttggaaactggcactaggtcaatctattctggcggtgttaaatttaaagg

SEQ ID NO: 79 - DNA, 300 bp, Artificial Sequence
  Helicobacter pylori vacA nucleic acid sequence
tttaaaagtggatgctcatacagctaattttaaaggtattgatactggtaatggtggtttcaacaccttagattttagtggtgttacaaacaaagtcaatatcaacaagctcattacagcttccactaatgtggccgttaaaaacttcaacattaatgaattgttggttaagattaatggggtgagtgtgggggaatacacttattttagcgaagatataggcagtcaatcgcgcatcaacaccgtgcgtttggaaactggcactaggtcaatctattctggcggtgttaaatttaaagg

SEQ ID NO: 80 - DNA, 300 bp, Artificial Sequence
  Helicobacter pylori vacA nucleic acid sequence
tttaaaggtggatgctcatacagctaattttaaaggtattgatacgggtaatggtggtttcaacaccttagattttagtggtgttacaggtaaggtcaatatcaacaagctcatcacggcttccactaatgtggccgttaaaaacaacaacattaatgaattggtggttaaaaccaatgggataagtgtgggggaatacactcattttagcgaagatataggcagtcaatcgcgcatcaataccgtgcgtttggaaacaggcactaggtcaatcttttctgggggtgtcaaatttaaaag

SEQ ID NO: 81 - DNA, 300 bp, Artificial Sequence
  Helicobacter pylori vacA nucleic acid sequence
tttaaaagtggatgctcatacagctaattttaaaggtattgatacgggtaatggtggtttcaacaccttagattttagtggtgttacaggtaaggtcaatatcaacaagctcattacggcttccactaatgtagccgttaaaaacttcaacattaatgaattgttggttaagaccaatggggtgagtgtgggggaatacactcattttagcgaagatataggcagtcaatcgcgcatcaacaccgtgcgtttggaaactggcactaggtcaatcttttctgggggtgtcaaatttaaaag

SEQ ID NO: 82 - DNA, 300 bp, Artificial Sequence
  Helicobacter pylori vacA nucleic acid sequence
tttaaaggtggatgctcatacagctaattttaaaggtattgatacgggtaatggtggtttcaacaccttagattttagtggtgttacaggtaaggtcaatatcaacaagctcatcacagcttccactaatgtggccgttaaaaacttcaacattaatgaattgattgttaaaaccaatgggataagtgtgggggaatacactcattttagcgaagatataggaagtcaatcgcgcatcaataccgtgcgtttggaaactggcactagatcaatcttttctgggggtgttaaatttaaagg

SEQ ID NO: 83 - DNA, 375 bp, Artificial Sequence
  Helicobacter pylori vacA nucleic acid sequence
tttaagagtggacgctcatacagcttattttaatggcaatatttatctgggaaaatccacgaatttaagagtgaatggccatagcgctcattttaaaaatattgatgccagcaagagcgataacgggctaaacactagcgctttggatttcagcggcgttacagacaaagtcaatatcaacaagctcactacatctgccactaatgtgaacgttaaaaactttgacgttaaggaattggtggttacaacccgtgttcagagttttgggcaatacactatttttggcgaaaatataggcgataagtctcgcattggtgtcgtgagtttgcaaacgggatatagcccggcctattctgggggcgttacttttaaaag

SEQ ID NO: 84 - DNA, 375 bp, Artificial Sequence
  Helicobacter pylori vacA nucleic acid sequence
cttaagagtggatgctcatacagcttattttaatggcaatatttatttgggaaaatccacgaatttaagagtgaatggccatagcgctcattttaaaaatattgatgccagtaagagcgataacgggctaaacactagtgctttggattttagcggcgttacagataaagtcaatatcaacaagctcactacatctgccactaatgtgaacgttaaaaactttgacattaaggaattggtggttacaacccgagttcaaagttttgggcaatacactatttttggcgaaaatataggcgataagtctcgcattggtgtcgttagtttgcaaacgggatatagcccggcctattctgggggcgttacttttaaaag

SEQ ID NO: 85 - DNA, 374 bp, Artificial Sequence
  Helicobacter pylori vacA nucleic acid sequence
tttaagagtggatgctcatacagcttattttaatggcaatatttatctgggaaaatccacgaatttaagagtgaatggccatagcgctcattttaaaaatattgatgccagtaagagcgataacgggctaaacactaccactttggatttcagcggcgttacagataaagtcaatatcaacaagctcactacatctgccactaatgtgaacattaaaaactttgacattaaggaattagtggttacaacccgagttcagagttttgggcaatacactatttttggcgaaaatataggcgataagctgcacattggtgtcgtgagtttgcaaacgggatatagcccagcctattctggggggcttacttttaaag

SEQ ID NO: 86 - DNA, 375 bp, Artificial Sequence
  Helicobacter pylori vacA nucleic acid sequence
tttaagagtggatgctcatacagcttattttaatggcaatatttatctgggaaaatccacgaatttaagagtgaatggccatagcgctcattttaaaaatattgatgccagtaagagcgataacgggctaaacactagctctttggatttcagtggcgttacagacaaagtcaatatcaacaagctcactacatctgccactaatgtgaacgttaaaaactttgacattaaggaattggtggttacaacccgcgttcagagttttgggcaatacactatttttggcgaaaatataggcgataagtctcgcattggtgtcgttagtttgcaaacgggatatagcccggcctattctgggggcgttacttttaaaag

SEQ ID NO: 87 - DNA, 365 bp, Artificial Sequence
  Helicobacter pylori vacA nucleic acid sequence
gatgctcatacagcttattttaatggcaatatttatctgggaaaatccacgaatttaagagtgaatggccatagcgctcattttaaaaatattgatgccagtaagagcgataacgggctaaacactagcgctttggatttyagcggcgttacagayaaagtcaatatcaacaagctcactacatctgccactaatgtgaacgttaaaaactttgacattaaggaattagtggttacaacccgagttcaaagttttgggcaatacactatttttggcgaaaatataggcgataagtctcgcattggtgtcgttagtttgcaaacgggatatagcccggcctattctgggggcgttacttttaaaag

SEQ ID NO: 88 - DNA, 375 bp, Artificial Sequence
  Helicobacter pylori vacA nucleic acid sequence
tttaagaggggatgctcatacagcttattttaatggcaatatttatttgggaaaatccacgaatttaagagtgaatggccatagcgctcattttaaaaatattgatgccagtaagagcgataacgggctaaacactagcgytttggattttagcggcgttacagayaaagtcaatatcaacaagctcactacatctgccactaatgtgaacrttaaaaactttgayattaaggaattggtggttacaacccgagttcaaagttttgggcaatacactatttttggcgaaaatataggcgatmagtctcgcattggtgtcgttagtttgcaaacgggatatagcccrgcctattctgggggcgttacttttaaaag

SEQ ID NO: 89 - DNA, 375 bp, Artificial Sequence
  Helicobacter pylori vacA nucleic acid sequence
tttaagcgtggatgctcatacagcttattttaatggtaatatttatctgggaaaatccacgaatttaagagtgaatggccatagcgctcattttaaaaatattgatgccacaaagagcgataacgggctaaacactagcgctttggatttcagcggcgttacagataaagtcaatatcaacaagctcactacatctgccactaacgtgaacattaaaaactttgacattaaggaattggtggttacaacccgagttcaaagttttgggcaatacactatttttggcgaaaatataggcgataagtctcgcattggtgtcgtgagtttgcaaacgggatatagcccggcctattctgggggcgttacttttaaaag

SEQ ID NO: 90 - DNA, 375 bp, Artificial Sequence
  Helicobacter pylori vacA nucleic acid sequence
tttaagagtggatgctcatacagcttattttaatggcaatatttatctgggaaaatccacgaatttaaaagtgaatggccatagcgctcattttaaaaatattgatgccagtaagagcgataatggtctaaacactagtgctttggatttgagcggcgttacagacaaagtcaatatcaacaagctcactacagctgccactaatgtgaacattaaaaactttgacattaaggaattagtggttacgacccgtgttcagagttttgggcaatacactatttttggcgaaaatataggagatcaatcgcgcattggtgtcgttagtttgcaaactggctatagcccggcctattctgggggcgttacttttaaaag

SEQ ID NO: 91 - DNA, 375 bp, Artificial Sequence
  Helicobacter pylori vacA nucleic acid sequence
cttaagagtggatgctcatacagcttattttaatggcaatatttatctgggaaaatccacgaatttaagagtgaatggccatagcgctcattttaaaaatattgatgctagtaagagcgataacgggctaaacactagcgctttggattttagcggcgttacagacaaagtcaatatcaacaagctcactacatctgccactaatgtgaacattaaaaactttgacattaaggaattggtggttacaacccgagttcaaagttttgggcaatacactatttttggcgaaaatataggcgataagtctcgcattggtgtcgtgagtttgcaaacgggatatagcccggcctattctgggggcgttacttttaaaag

SEQ ID NO: 92 - DNA, 449 bp, Artificial Sequence
  Helicobacter pylori cagA nucleic acid sequence
atgactaacgaaaccattaaccaacaaccacaaagcgaagcggcttttaacccgcagcaatttatcaataatcttcaagtagcttttcttaaagttgataacgctgtcgcttcatacgatcctgatcaaaaaccaatcgttgataagaacgatagggataataggcaagcttttgatggaatctcgcaattaagggaagaatactccaataaagcgatcaaaaatcctaccaaaaagaatcagtatttttcagactttatcaataagagcaatgatttaatcaacaaagacgctctcattgatgtagaatcttccacaaagagctttcagaaatttggggatcagcgttaccgaattttcacaagttgggtgtcccatcaaaacgatccgtctaaaatcaacacccgatcgatccgaaattttatggaaaatatcatacaaccccctat

SEQ ID NO: 93 - DNA, 449 bp, Artificial Sequence
  Helicobacter pylori cagA nucleic acid sequence
atgactaacgaaaccattaaccaacaaccacaaaccgaagcggcttttaacccgcagcaatttatcaataatcttcaagtagcttttcttaaagttgataacgctgtcgcttcatacgatcctgatcaaaaaccaatcgttgataagaacgatagggataacaggcaagcttttgatggaatctcgcaattaagggaagaatactccaataaagcgatcaaaaatcctaccaaaaagaatcagtatttttcagactttatcaataagagcaatgatttaatcaacaaagacgctctcattgatgtagaatcttccacaaagagctttcagaaatttggggatcagcgttaccgaattttcacaagttgggtgtcccatcaaaacgatccgtctaaaatcaacacccgatcgatccgaaattttatggaaaatatcatacaaccccctat

SEQ ID NO: 94 - DNA, 449 bp, Artificial Sequence
  Helicobacter pylori cagA nucleic acid sequence
atggctaacgaaactattaaccaacaaccacaaaccgaagcggcttttaacccgcagcaatttatcaataatcttcaagtagcttttcttaaagttgataacgctgtcgcttcatacgatcctgatcaaaaaccaatcgttgataagaacgatagggataacaggcaagcttttgatggaatctcgcaattaagggaagaatactccaataaagcgatcaaaaatcctaccaaaaagaatcagtatttttcagactttatcaataagagcaatgatttaatcaacaaagacgctctcattgatgtagaatcttccacaaagagctttcagaaatttggggatcagcgttaccgaattttcacaagttgggtgtcccatcaaaacgatccgtctaaaatcaacacccgatcgatccgaaattttatggaaaatatcatacaaccccctat

SEQ ID NO: 95 - DNA, 449 bp, Artificial Sequence
  Helicobacter pylori cagA nucleic acid sequence
atgactaacgaaaccattaaccaacaaccacaaaccgaagcggcttttaacccgcagcaatttatcaataatcttcaagtggcttttcttaaagttgataacgctgtcgcttcatacgatcctgatcaaaaaccaatcgttgataagaacgatagggataataggcaagcttttgatggaatctcgcaattaagggaagaatactccaataaagcgatcaaaaatcctaccaaaaagaatcagtatttttcagactttatcaataagagcaatgatttaatcaacaaagacgctctcattgatgtagaatcttccacaaagagctttcagaaattttgggatcagcgttaccgaattttcacaagttgggtgtcccatcaaaacgatccgtctaaaatcaacacccgatcgatccgaaatttcatggaaaatatcatacaaccccctat

SEQ ID NO: 96 - DNA, 449 bp, Artificial Sequence
  Helicobacter pylori cagA nucleic acid sequence
atgactaacgaaaccattaaccaacaaccacaaaccgaagcggcttttaacccgcagcaatttatcaataatcttcaagtggcttttcttaaagttgataacgctgtcgcttcatacgatcctgatcaaaaaccaatcgttgataagaacgatagggataacaggcaagcttttgatggaatctcgcaattaagggaagaatactccaataaagcgatcaaaaatcctaccaaaaagaatcagtatttttcaaactttatcaataagagcaatgatctaatcaacaaagacaatctcattgatgtagaatcttccaaaaagagctttcagaaatttggggatcagcgttaccgaattttcacaagttgggtgtcccatcaaaacgatccgtctaaaatcaacacccgatcgatccgaaattttatggaaaatatcatacaaccccctat

SEQ ID NO: 97 - DNA, 449 bp, Artificial Sequence
  Helicobacter pylori cagA nucleic acid sequence
atgactaacgaaaccattaaccaacaaccacaaaccgaagcggcttttaacccgcagcaatttatcaataatcttcaagtagcttttcttaaagttgataacgctgtcgcttcatacgatcctgaccaaaaaccaatcgttgataagaacgatagggataacaggcaagcttttgatagaatctcacaattaagggaggaatactccaataaagcgatcaaaaatcctaccaaaaagaatcagtatttttcagactttatcgataagagcaacgatttaatcaacaaagacgctctcattgatgtagaatcttccacaaagagctttcagaaatttggggatcagcgttaccgaattttcacaagttgggtgtcccatcaaaacgatccgtctaaaatcaacacccgatcgatccgaaattttatggaaaatatcatacaaccccctat

SEQ ID NO: 98 - DNA, 449 bp, Artificial Sequence
  Helicobacter pylori cagA nucleic acid sequence
atgactaacgaaaccattaaccaacaaccacaaaccgaagcggcttttaacccgcagcaatttatcaataatcttcaagtggcttttcttaaagttgataacgctgtcgcttcatacgatcctgatcaaaaaccaattattgataagaacgatagggataacaggcaagcttttgatggaatctcgcaattaagggaagaatattccaataaagcgatcaaaaatcctaccaaaaagaatcagtatttttcagactttatcgataagagcaatgatttaatcaacaaagacaatctcattgatgtagaatcttccacaaagagctttcagaaatttggggatcagcgttaccgaattttcacaagttgggtgtcccatcaaaacgatccgtctaaaatcaacacccgatcgatccgaaattttatggaaaatatcatacaaccccctat

SEQ ID NO: 99 - DNA, 449 bp, Artificial Sequence
  Helicobacter pylori cagA nucleic acid sequence
atgactaacgaaaccattaaccaacaaccacaaaccgaagcggcttttaacccgcagcaatttatcaataatcttcaagtagcttttcttaaagttgataatgctgtcgcttcatacgattctgatcaaaaaccaatcattgataagaacgatagggataacaggcaagcttttgatagaatctcgcaattaagggaagaatactccaataaagcgatcaaaaatcctaccaaaaagaatcagtatttttcagactttatcgataagagcaacgatttaatcaacaaagacaatctcattgatgtagaatcttccacaaagagctttcagaaatttggggatcagcgttaccgaattttcacaagttgggtgtcccatcaaaatgatccgtctaaaatcaacacccgatcgatccgaaattttatggaaaatatcatacaaccccctat

SEQ ID NO: 100 - DNA, 449 bp, Artificial Sequence
  Helicobacter pylori cagA nucleic acid sequence
atgactaacgaaactattgaccaacaaccacaaaccgaagcggcttttaacccgcagcaatttattaataatcttcaggtagcttttcttaagcttgataacgctgtcgcttcatttgatcctgatcaaaaaccaatcgttgataagaatgatagggataacaggcaagcttttgatggaatctcgcaattaagggaagaatactccaataaagcgatcaaaaatcctaccaaaaagaatcagtatttttcagactttatcaataagagcaatgatttaatcaacaaagacgctctcattgatgtagaatcttccacaaagagctttcagaaatttggggatcagcgttaccgaattttcacaagttgggtgtcccatcaaaacgatccgtctaaaatcaacacccgatcgatccaaaattttatggaaaatatcatacaaccccctat

SEQ ID NO: 101 - DNA, 449 bp, Artificial Sequence
  Helicobacter pylori cagA nucleic acid sequence
atgactaacgaaactattgaccaacaaccacaaactgaagcggcttttaacccgcagcaatttatcaataatcttcaagtggcttttcttaagcttgataacgctgtcgcttcatttgatcctgatcaaaaaccaatcgttgataagaacgatagggataacaggcaagcttttgatggaatctcgcaattaagggaagaatactccaataaagcgatcaaaaatcctaccaaaaagaatcagtatttttcagactttatcaataagagcaatgatttaatcaacaaagacgctctcattgatgtagaatcttccacaaagagctttcagaaatttggggatcagcgttaccgaattttcacaagttgggtgtcccatcaaaacgatccgtctaaaatcaacacccgatcgatccgaaattttatggaaaatatcatacaaccccctat

SEQ ID NO: 102 - DNA, 449 bp, Artificial Sequence
  Helicobacter pylori cagA nucleic acid sequence
atgactaacgaaactattaaccaacagccacaaaccgaagcggcttttaacccgcagcaatttatcaataatcttcaagtagcttttcttaagcttgataacgctgtcgcttcatttgatcctgatcaaaaaccaatcgttgataagaacgatagggataataggcaggcttttgatggaatctcgcaattaagggaagaatactccaataaagcgatcaaaaatcctaccaaaaagaatcagtatttttcagactttatcaataagagcaatgatttaatcaacaaagacaatctcattgatgtagaatcttccacaaagagctttcagaaatttggggatcagcgttaccgaattttcacaagttgggtgtcccatcaaaacgatccgtctaaaatcaacacccgatcgatccgaaattttatggaaaatatcatacaaccccctat

SEQ ID NO: 103 - DNA, 449 bp, Artificial Sequence
  Helicobacter pylori cagA nucleic acid sequence
atgactaacgaaaccattaaccaacaaccacaaaccgaagcggcttttaacccgcagcaatttatcaataatcttcaagtggcttttcttaagcttgataatgctgttgcttcatttgatcctgatcaaaaaccaatcgttgataagaacgatagggataacaggcaagcttttgatggaatctcgcaattaagggaagaatactccaataaagcgatcaaaaatcctaccaaaaagaatcagtatttttcagactttatcgataagagcaacgatttaatcaacaaagacaatctcattgatgtagaatcttccacaaagagctttcagaaatttggggatcagcgttaccgaattttcacaagttgggtgtcccatcaaaacgatccgtctaaaatcaacacccgatcgatccgaaattttatggaaaatatcatacaaccccctat

SEQ ID NO: 104 - DNA, 449 bp, Artificial Sequence; identical to SEQ ID NO: 95
  Helicobacter pylori cagA nucleic acid sequence

SEQ ID NO: 105 - DNA, 449 bp, Artificial Sequence
  Helicobacter pylori cagA nucleic acid sequence
atgactaacgaaactattgatcaacaaccacgaaccgaagcggcttttaacccgcagcaatttatcaataatcttcaagtagcttttcttaaagttgataacgttgtcgcttcatttgatcctaatcaaaaaccaatcgttgataagaacgatagggataacaggcaagcttttgatggaatctcgcaattaagggaagaatactccaataaagcgatcaaaaatcctgccaaaaagaatcagtatttttcagactttatcaataagagcaatgatctaatcaacaaagacaatctcattgatgtagaatcttccacaaagagctttcagaaatttggggatcagcgttaccaaattttcacaagttgggtgtcccatcaaaacgatccgtctaaaatcaacacccgatcgatccgaaattttatggaaaatatcatacaaccccctat

SEQ ID NO: 106 - DNA, 449 bp, Artificial Sequence
  Helicobacter pylori cagA nucleic acid sequence
atgactaacgaaaccattaaccaacaaccacaaaccgaagcggcttttaacccgcagcaatttatcaataatcttcaagtggcttttattaaagttgataatgttgtcgcttcatttgatcctgatcaaaaaccaatcgttgataagaatgatagggataataggcaagcttttgagaaaatctcgcagctaagggaggaattcgctaataaagcgatcaaaaatcctgccaaaaagaatcagtatttttcaagctttatcagtaagagcagtgatttaatcaacaaagacagtctcattgatacaggttcttccataaagagctttcagaaatttgggactcagcgttaccaaatttttatgaattgggtgtcccatcaaaaagatccatctaaaatcaacacccaaaaaatccgaggttttatggaaaatatcatacaaccccctat

SEQ ID NO: 107 - DNA, 464 bp, Artificial Sequence
  Helicobacter pylori cagA nucleic acid sequence
atgactaacgaaactattgatcaaacaagaacaccagaccaaacacaaagccaaacagcttttgatccgcaacaatttatcaataatattcaagtggcttttcttaaagttgataacgctgtcgcttcatttgatcctgatcaaaaaccaatcgttgataagaatgatagggataacaggcaagcttttgagaaaatctcgcagctaagggaggaattcgctaataaagcgatcaaaaatcctgccaaaaagaatcagtatttttcaagctttatcagtaagagcagtgatttagtcaacaaagacagtctcattgatacaggttcttccataaagagctttcagaaatttgggactcagcgttaccaaatttttatgaattgggtgtcccatcaaaaagatccatctaaaatcaacacccaaaaaatccaagattttatggaaaatatcatacaaccccctat

SEQ ID NO: 108 - DNA, 464 bp, Artificial Sequence
  Helicobacter pylori cagA nucleic acid sequence
atgactaatgaaaccattgatcaaacaacaacaccagatcaaacaccaaatcaaacagattttgttccgcaacgatttatcaataatcttcaagtagcttttattaaagttgataacgctgtctcttcatttgatcctgatcaaaaaccaatcgttgataagaatgatagggataacaggcaagcttttgagaaaatctcgcaactaagggaagaatacgccaataaagcgatcaaaaatcctgccaaaaagaatcagtatttttcagactttatcaataagagcaatgatttgatcaacaaagacaatctcattgctgtagattcttccgtagagacctttaagaaatttggggatcagcgttaccaaatttttacgaattgggtgtcccttcaaaaagatccgtctaaaatcaacacccgacaaatccgaaattttatggaaaatatcatacaaccccctat

SEQ ID NO: 109 - DNA, 464 bp, Artificial Sequence
  Helicobacter pylori cagA nucleic acid sequence
atgactaacgaaaccattgatcaaacaacaacaccagatcaaacaccaaaccaaacggattttgttccgcaacgatttatcaataatcttcaagtagctttccttaaagttgatagcgctgtcgcttcatttgatcctgatcaaaaaccaatcgttgataagaatgatagggataacaggcaagcttttgagaaaatctcgcaactaagggaagaatacgccaataaagcgatcaaaaatcctgccaaaaagaatcagtatttttcagactttatcaataagagcaatgatttgatcaacaaagacaatctcattgctgtagattcttctgtagagagctttaagaaatttggggatcagcgttaccaaatttttacgagttgggtgtcccttcaaaaagatccgtctcaaatcaacacccgacaaatccgaaattttatggaaaatatcatacaaccccctat

SEQ ID NO: 110 - DNA, 464 bp, Artificial Sequence
  Helicobacter pylori cagA nucleic acid sequence
atgactaacgaaaccattgatcaaacaacaacaccagatcaaacaccaagccaaacagattttgttccgcaacgatttatcaataatcttcaagtagcttttcttaaagttgataacgctgtcgcttcatttgatcctgatcaaaaaccaatcgttgataagaatgatagggataacaggcaagcttttgagaaaatctcgcaactaagggaagaatacgccaataaagcgatcaaaaatcctgccaaaaagaatcagtatttttcagactttatcaataagaccaatgatttgatcaacaaagacaatctcattgctgtagattcttccgtagatagctttaagaaatttggggatcagcgttaccaaatttttacgagttgggtgtcccttcaaaaagatccgtctaaaatcaacacccaacaaatccgaaattttatggaaaatatcatacaaccccctat

SEQ ID NO: 111 - DNA, 464 bp, Artificial Sequence
  Helicobacter pylori cagA nucleic acid sequence
atgactaacgaaaccattgatcaaacaacaacaccagatcaaacaccaaatcaaacagattttgttccgcaacgatttatcaataatcttcaagtagcttttattaaagttgatgacgctgtcgcttcatttgatcccgatcaaaaaccaatcgttgataagaatgatagggataacaggcaagcttttgagaaaatctcgcaactaagggaagaatacgccaataaagcgatcaaaaatcccaccaaaaagaatcagtatttttcagactttatcaataagaccaatgatttgatcaacaaagacaatctcattgctgtagattcttccgtagagagctttaagaaatttggggatcagcgttaccaaatttttacgagttgggtgtcccttcaaaaagatccgtctaaaatcaacacccaacaaatccgaaattttatggaaaatatcatacaaccccctat

SEQ ID NO: 112 - DNA, 464 bp, Artificial Sequence
  Helicobacter pylori cagA nucleic acid sequence
atgactaacgaaaccattgatcaaacaacaacaccagatcaaacaccaaatcaaacagattttgttccgcaacgatttatcaataatcttcaagtagcttttattaaagttgataacgctgttgcttcatttgatcccgatcaaaaaccaatcgttgataagaatgatagggataacaggcaagcttttgagaaaatctcgcaactaagggaagaatacgccaataaagcgatcaaaaatcctgccaaaaagaatcagtatttttcagactttatcaataagagcaatgatttgatcaacaaagacaatctcattgctgtagattcttccgtagatagctttaagaaatttggggatcagcgttaccaaatttttacgagttgggtgtcccttcaaaaagatccgtctcaaatcaacacccaacaaatccaaaattttatggaaaatatcatacaaccccctat

SEQ ID NO: 113 - DNA, 464 bp, Artificial Sequence
  Helicobacter pylori cagA nucleic acid sequence
atgactaacgaaaccattgatcaaacaacaacaccagatcaaacactaaaccaaacggattttgttccgcaacgatttatcaataatcttcaagtagcttttattaaagttgataacgctgtcgctttatttgatcccgatcaaaaaccaatcgttgataagaatgatagggataacaggcaagcttttgagaaaatctcgcaactaagggaagaatacgccaataaagcgatcaaaaatcccaccaaaaagaatcagtatttttcagactttatcaataagagcaatgatttgatcaacaaagacaatctcattgctgtagattcttccgtagatagctttaagaaatttggggatcagcgttaccaaatttttacgagttgggtgtcccttcaaaaagatccgtctcaaatcaacacccgacaaatccgaaattttatggaaaatatcatacaaccccctat

SEQ ID NO: 114 - DNA, 464 bp, Artificial Sequence
  Helicobacter pylori cagA nucleic acid sequence
atgactaacgaaaccattgatcaaacaataacaccagatcaaacaccaaaccaaacggattttgttccgcaacgatttatcaataatcttcaagtagcttttatcaaagttgataacgctgtcgcttcatttgatcctgatcaaaaaccaatcgttgataagaatgatagggataacaggcaagcttttgagaaaatctcgcaattaagggaagaatacgccaataaagcgatcaaaaatcctgccaaaaagaatcagtattttttagactttatcaataagagcaatgatttgatcaacaaagacaatctcattgctgtagattcttccgtagatagctttaagaaatttggggatcagcgttaccaaatttttacgagttgggtgtcccttcaaaaagatccgtctaaaatcaacacccaacaaatccgaaattttatggaaaatatcatacaaccccctat

SEQ ID NO: 115 - DNA, 132 bp, Artificial Sequence
  Helicobacter pylori vacA nucleic acid sequence
ccctattatctctctcgctctagtgggggtgttaatgggtaccgaactaggggctaacacgccaaacgatcccatacacagcgagagtcgcgccttttttacaaccgtgatcattccagccattgttggggg

SEQ ID NO: 116 - DNA, 132 bp, Artificial Sequence
  Helicobacter pylori vacA nucleic acid sequence
ccctattatctctctcgctctagtgggggtgttaatgggtaccgaactaggggctaacacgccaaacgatcccatacacagcgagagtcgcgcctttttcacaaccgtgatcattccagccattgttggagg

SEQ ID NO: 117 - DNA, 132 bp, Artificial Sequence
  Helicobacter pylori vacA nucleic acid sequence
ccctattatctctctcgctctagtgggggtgttaatgggcaccgaactaggggctaatacgccaaacgatcccatacacagcgagagtcgcgcctttttcacaaccgtgatcattccagccattgttggggg

SEQ ID NO: 118 - DNA, 132 bp, Artificial Sequence; identical to SEQ ID NO: 117
  Helicobacter pylori vacA nucleic acid sequence

SEQ ID NO: 119 - DNA, 132 bp, Artificial Sequence
  Helicobacter pylori vacA nucleic acid sequence
ccctattatctctctcgctctagtgggggtgttaatgggcactgaactaggggctaacacgccaaacgatcccatacacagcgagagtcgcgccttttttacaaccgtgatcattccagccattgttggggg

SEQ ID NO: 120 - DNA, 132 bp, Artificial Sequence
  Helicobacter pylori vacA nucleic acid sequence
ccctattatctctctcgctctagtgggggtgttaatgggcaccgaactaggggctaacacgccaaatgatcccatacacagcgagagtcgcgcctttttcacaacygtgatcattccagccattgttggggg

SEQ ID NO: 121 - DNA, 132 bp, Artificial Sequence
  Helicobacter pylori vacA nucleic acid sequence
ccctattatctctctcgctttagtgggggtrttaatgggcaccgaactaggggctaacacgccaaacgatcccatacacagcgagagtcgcgcctttttcacaaccgtgatcattccagccattgttggggg

SEQ ID NO: 122 - DNA, 132 bp, Artificial Sequence
  Helicobacter pylori vacA nucleic acid sequence
ccctattatttctctcgctttagtgggggtgttaatgggcaccgaactaggggctaacacgccaaacgatcccatacacagcgagagtcgcgccttttttacaaccgtgatcattccagccattgttggggg

SEQ ID NO: 123 - DNA, 132 bp, Artificial Sequence
  Helicobacter pylori vacA nucleic acid sequence
ccctattatctctctcgctctagtgggggcattaatgagtaccgaactaggggctaacacgccaaatgatcccatacacagcgagagtcgcgccttttttacaaccgtgatcattccagccattgttggggg

SEQ ID NO: 124 - DNA, 132 bp, Artificial Sequence
  Helicobacter pylori vacA nucleic acid sequence
ccctattatctctctcgctttagtgggggtattaataggcaccgaactaggggctaatacgccaaatgatcccatacacagcgagagtcgtgcttttttcacaaccgtgatcattccagccattgttggggg

SEQ ID NO: 125 - DNA, 132 bp, Artificial Sequence
  Helicobacter pylori vacA nucleic acid sequence
ccctattatctctctcgctctagtgggggtgttaataagcaccgaactaggggctaacacgccaaatgatcccatacacagcgagagtcgcgcctttttcacaacggggatcattccagccattgttggggg

SEQ ID NO: 126 - DNA, 105 bp, Artificial Sequence
  Helicobacter pylori vacA nucleic acid sequence
ccctttagtttctctcgctttagtggggttattggtcagcatcacaccacaaaaaagtcatgctgccttctttacaaccgtgatcattccagccattgttggggg

SEQ ID NO: 127 - DNA, 105 bp, Artificial Sequence; identical to SEQ ID NO: 126
  Helicobacter pylori vacA nucleic acid sequence

SEQ ID NO: 128 - DNA, 105 bp, Artificial Sequence
  Helicobacter pylori vacA nucleic acid sequence
ccctttagtttctctcgctttagtggggttattggtcagcatcacaccgcaaaaaagtcatgctgccttctttacaaccgtgatcattccagccattgttggggg

SEQ ID NO: 129 - DNA, 105 bp, Artificial Sequence
  Helicobacter pylori vacA nucleic acid sequence
ccctctagtttctctcgctttagtggggttattggtcagcatcacaccgcaaaaaagtcatgccgccttctttacaaccgtgatcattccagccattgttggggg

SEQ ID NO: 130 - DNA, 105 bp, Artificial Sequence
  Helicobacter pylori vacA nucleic acid sequence
ccctctagtttctctcgctttagtggggttattggtcagcatcacaccgcaaaaaagtcatgccgccttctttacaaccgtgatcattccadccattgttggggg

SEQ ID NO: 131 - DNA, 105 bp, Artificial Sequence
  Helicobacter pylori vacA nucleic acid sequence
ccctttagtttctctcgctttagtggggttattggtcagcatcacaccgcaaaaaagtcatgccgccttctttacaaccgtgatcattccggccattgttggggg

SEQ ID NO: 132 - DNA, 105 bp, Artificial Sequence
  Helicobacter pylori vacA nucleic acid sequence
ccctttagtttctctcgctttagtggggttattggtcagcatcacaccgcaaaaaagtcatgccgccttctttacaaccgtgatcattccagccattgttggggg

SEQ ID NO: 133 - DNA, 105 bp, Artificial Sequence
  Helicobacter pylori vacA nucleic acid sequence
ccctttagtttctctcgctttagtggggctattggtcagcatcacaccgcaaaaaagtcatgccgccttctttacaaccgtgatcattccagccattgttggggg

SEQ ID NO: 134 - DNA, 105 bp, Artificial Sequence
  Helicobacter pylori vacA nucleic acid sequence
ccctttagtttctctcgctttagtggggttattggtcagcatcacaccgcaaaaaagtcatgccgcctttttcacaaccgtgatcattccggccattgttggggg

SEQ ID NO: 135 - DNA, 105 bp, Artificial Sequence
  Helicobacter pylori vacA nucleic acid sequence
ccctttagtttctctcgctttagtggggctattggtcagcatcacaccgcaaaaaagtcatgccgccttctttacaaccgtgattatcccggccattgttggggg

SEQ ID NO: 136 - DNA, 105 bp, Artificial Sequence
  Helicobacter pylori vacA nucleic acid sequence
ccctttagtttctctcgctttagtggggctattggtcagcatcacaccacaaaaaagtcatgccgccttttttacaaccgtgatcattccagccattgttgggag

SEQ ID NO: 137 - DNA, 105 bp, Artificial Sequence
  Helicobacter pylori vacA nucleic acid sequence
ccctttagtttctctcgctttagtggggctattggtcagcatcacaccacaaaaaagtcatgccgccttttttacaaccgtgatcattccagccattgttggagg

SEQ ID NO: 138 - DNA, 105 bp, Artificial Sequence
  Helicobacter pylori vacA nucleic acid sequence
ccctctggtatctctcgctttagtggggctattggtcagcatcacaccgcaaaaaagtcatgccgccttctttacaaccgtgaatattccagcmattgttggggg

SEQ ID NO: 139 - DNA, 105 bp, Artificial Sequence
  Helicobacter pylori vacA nucleic acid sequence
ccctctggtttctctcgctttagtggggctattggtcagcatcacaccgcaaaaaagtcatgccgcctttttcacaaccgtgatcattccggccattgttggggg

SEQ ID NO: 140 - DNA, 105 bp, Artificial Sequence
  Helicobacter pylori vacA nucleic acid sequence
ccctctggtttctctcgctttagtggggctattggtcagcatcacaccgcaaaaaagtcatgccgccttctttacaaccgtgatcattccagcmattgttggggg

SEQ ID NO: 141 - DNA, 105 bp, Artificial Sequence
  Helicobacter pylori vacA nucleic acid sequence
ccctctggtttctctcgctttagtggggctattggtcagcatcacaccgcaaaaaagtcatgccgccttctttacaaccgtgatcattccagccattgttggggg

SEQ ID NO: 142 - DNA, 105 bp, Artificial Sequence
  Helicobacter pylori vacA nucleic acid sequence
ccctctggtttctctcgctttagtggggctattggtcagcatcacaccacaaaaaagtcatgccgccttctttacaaccgtgatcattccagchattgttggggg

SEQ ID NO: 143 - DNA, 105 bp, Artificial Sequence
  Helicobacter pylori vacA nucleic acid sequence
ccctctggtttctctcgctttagtggggctattggtcagcatcacaccacaaaaaagtcatgccgccttctttacaaccgtgatcattccagcyattgttggagg

SEQ ID NO: 144 - DNA, 105 bp, Artificial Sequence
  Helicobacter pylori vacA nucleic acid sequence
ccctctggtttctctcgctttagtggggttattggtcagcatcacaccgcaaaaaagtcatgccgccttctttacaaccgtgatcattccagccattgttggagg

SEQ ID NO: 145 - DNA, 105 bp, Artificial Sequence
  Helicobacter pylori vacA nucleic acid sequence
ccctttagtttctctcgctttagtggggttattggttagcatcacaccgcaaaaaagtcatgccgcctttttcacaaccgtgattattccagccattggttgggg

SEQ ID NO: 146 - DNA, 105 bp, Artificial Sequence
  Helicobacter pylori vacA nucleic acid sequence
ccctctggtttctctcgctttagtggggctattggtcagcatcacaccacaaaaaagtcatgccgccttctttacaaccgtgatcattccagccatcgttccccc

SEQ ID NO: 147 - DNA, 105 bp, Artificial Sequence
  Helicobacter pylori vacA nucleic acid sequence
ccctttagtttctcctgttttagcaggagcgttgattagctccataccgcaacaaagtcatgccgcctttttcacaaccgtgatcattccagccattgttggggg

SEQ ID NO: 148 - DNA, 105 bp, Artificial Sequence
  Helicobacter pylori vacA nucleic acid sequence
ccctttagtttctctcgttttagcaggagcgttgattagtgccataccgcaagaaagtcatgccgccttttttacaaccgtaatcattccagctattgttggggg

SEQ ID NO: 149 - DNA, 105 bp, Artificial Sequence
  Helicobacter pylori vacA nucleic acid sequence
ccctttagtttctctcgttttagcaggagcgttgattagcgccataccgcaagagagtcatgccgcctttttcacgaccgtgatcattccagccattgttggggg

SEQ ID NO: 150 - DNA, 105 bp, Artificial Sequence
  Helicobacter pylori vacA nucleic acid sequence
ccctttagtttctctcgttttagcaggagcgtttattagcgccataccgcaagagagtcatgccgcctttttcacgaccgtgatcattccagccattgttggggg

SEQ ID NO: 151 - DNA, 105 bp, Artificial Sequence
  Helicobacter pylori vacA nucleic acid sequence
ccctttagtttctctcgttttagcaggagcgttgattagcgccataccgcaagaaagttatgccgcctttttcacgaccgtgatcattccagccattgttggggg

SEQ ID NO: 152 - DNA, 105 bp, Artificial Sequence
  Helicobacter pylori vacA nucleic acid sequence
ccctttagtttctctcgttttagcaggagcgttgattagcgccataccgcaacaaagtcatgccgcctttttcacaaccgtgatcattccagccattgttggggg

SEQ ID NO: 153 - DNA, 105 bp, Artificial Sequence
  Helicobacter pylori vacA nucleic acid sequence
ccctattatttctctcgttttagcaggagcgttgattagctccataccgcaagaaagtcatgccgcctttttcacgaccgtgatcattccagccattgttggggg

SEQ ID NO: 154 - DNA, 105 bp, Artificial Sequence
  Helicobacter pylori vacA nucleic acid sequence
ccctttagtttctctcgttttagcagnagcgttnattagtgccataccgcaananagtcatgccgcctttttcacnaccgtnatcattccagccattgttggggg

SEQ ID NO: 155 - DNA, 105 bp, Artificial Sequence
  Helicobacter pylori vacA nucleic acid sequence
ccctttggtttctctcgttttagcaggagcgttgattagcgccataccgcaacaaagtcatgccgcctttttcacaaccgtgatcattccagccattgtgggggg

SEQ ID NO: 156 - DNA, 105 bp, Artificial Sequence; identical to SEQ ID NO: 152
  Helicobacter pylori vacA nucleic acid sequence

SEQ ID NO: 157 - DNA, 105 bp, Artificial Sequence; identical to SEQ ID NO: 152
  Helicobacter pylori vacA nucleic acid sequence

SEQ ID NO: 158 - DNA, 105 bp, Artificial Sequence
  Helicobacter pylori vacA nucleic acid sequence
ccctttagtttctctcgttttagcaggagcgttgattagctccataccgcaacaaagtcatgccgcctttttcacaaccgtgatcattccagccattgttggggg

SEQ ID NO: 159 - DNA, 105 bp, Artificial Sequence
  Helicobacter pylori vacA nucleic acid sequence
ccctttagtttctcttgttttagcaggagcgttgattagcgccataccgcaacaaagtcatgccgcctttttcacgaccgtgatcattccagccattgttggggg

SEQ ID NO: 160 - DNA, 105 bp, Artificial Sequence; identical to SEQ ID NO: 159
  Helicobacter pylori vacA nucleic acid sequence

SEQ ID NO: 161 - DNA, 105 bp, Artificial Sequence
  Helicobacter pylori vacA nucleic acid sequence
ccctttggtttctctcgttttagcaggagcgttgattagcgccataccgcaacaaagtcatgccgcctttttcacaaccgtgatcattccagcvattgtggggag

SEQ ID NO: 162 - DNA, 105 bp, Artificial Sequence; identical to SEQ ID NO: 149
  Helicobacter pylori vacA nucleic acid sequence

SEQ ID NO: 163 - DNA, 105 bp, Artificial Sequence
  Helicobacter pylori vacA nucleic acid sequence
ccctttagtttctctcgttttagcaggagcgttgattagtgccataccgcaagagagtcatgccgcctttttcacgaccgtgatcattccagccattgttggggg

SEQ ID NO: 164 - DNA, 105 bp, Artificial Sequence; identical to SEQ ID NO: 163
  Helicobacter pylori vacA nucleic acid sequence

SEQ ID NO: 165 - DNA, 105 bp, Artificial Sequence
  Helicobacter pylori vacA nucleic acid sequence
ccctttagtttctctcgttttagcaggagcgttggttagtgccataccgcaagagagtcatgccgcctttttcacgaccgtgatcattccagccattgttggggg

SEQ ID NO: 166 - DNA, 105 bp, Artificial Sequence
  Helicobacter pylori vacA nucleic acid sequence
ccctttagtttctctcgttttagcaggagcgttgattagtgccataccgcaagagagtcatgccgcctttttcacaaccgtgatcattccarccattgttggggg

SEQ ID NO: 167 - DNA, 105 bp, Artificial Sequence
  Helicobacter pylori vacA nucleic acid sequence
ccctttagtttctcttgttttagcaggagcgttgattagcgccataccgcaagagagtcatgccgcctttttcacaaccgtaatcattccagccattgttggggg

SEQ ID NO: 168 - DNA, 105 bp, Artificial Sequence
  Helicobacter pylori vacA nucleic acid sequence
ccctttagtttctcttgttttagcaggagcgttgattagcgccataccgcaagagagtcatgccgcctttttcacgaccgtaatcattccagccattgttggggg

SEQ ID NO: 169 - DNA, 105 bp, Artificial Sequence
  Helicobacter pylori vacA nucleic acid sequence
ccctttagtttctctcgttttagcaggagcgttgattagcgccataccgcaagagagtcatgccgcctttttcacaaccgtnatcattccagccattgttggggg

SEQ ID NO: 170 - DNA, 105 bp, Artificial Sequence
  Helicobacter pylori vacA nucleic acid sequence
ccctttggtttctctcgttttagcaggagcgttgattagctccataccgcaagagagtcatgccgcctttttcacacccgtgatcattccagccattgttggggg

SEQ ID NO: 171 - DNA, 105 bp, Artificial Sequence
  Helicobacter pylori vacA nucleic acid sequence
ccctttggtttctctcgttttagcaggagcgttgattagctccataccgcaagagagtcatgccgcctttttcacaaccgtgatcattccagccattgttggggg

SEQ ID NO: 172 - DNA, 105 bp, Artificial Sequence; identical to SEQ ID NO: 171
  Helicobacter pylori vacA nucleic acid sequence

SEQ ID NO: 173 - DNA, 105 bp, Artificial Sequence; identical to SEQ ID NO: 171
  Helicobacter pylori vacA nucleic acid sequence

SEQ ID NO: 174 - DNA, 105 bp, Artificial Sequence
  Helicobacter pylori vacA nucleic acid sequence
ccctttagtttctctcgttttagcaggagcgttgattagctccataccgcaagagagtcatgccgcctttttcacaaccgtgatcattccagccattgttggggg

SEQ ID NO: 175 - DNA, 105 bp, Artificial Sequence
  Helicobacter pylori vacA nucleic acid sequence
ccctttagtttctctcgttttagcannagcgttgattagcrccataccgcaagagagtcatgccgccttttttacaaccgtgatcattccagccattgttggggg

SEQ ID NO: 176 - DNA, 105 bp, Artificial Sequence
  Helicobacter pylori vacA nucleic acid sequence
ccctttagtttctcttgttttagcaggagcgttgattagcgccataccgcaacaaagtcatgccgcctttttcacgaccgtnatcattccagccattgttnnnng

SEQ ID NO: 177 - DNA, 105 bp, Artificial Sequence
  Helicobacter pylori vacA nucleic acid sequence
ccctctggtttctctggttttagcaggagcgttgattagcatcacaccacaacaaagtcatgccgcctttttcacgaccgtgatcattccagccattgttggggg

SEQ ID NO: 178 - DNA, 105 bp, Artificial Sequence
  Helicobacter pylori vacA nucleic acid sequence
ccctctggtttctctcgctttagtaggagcattggtcagcatcacaccgcaacaaagtcatgccgcctttttcacaaccgtgatcattccagccattgttggggg

SEQ ID NO: 179 - DNA, 105 bp, Artificial Sequence; identical to SEQ ID NO: 178
  Helicobacter pylori vacA nucleic acid sequence

SEQ ID NO: 180 - DNA, 105 bp, Artificial Sequence
  Helicobacter pylori vacA nucleic acid sequence
ccctctggtttctctcgctttagtaggagcattggtcagcatcacaccgcaacaaagtcatgccgcctttttcacaaccgtgatcattccagccattgttggagg

SEQ ID NO: 181 - DNA, 105 bp, Artificial Sequence
  Helicobacter pylori vacA nucleic acid sequence
ccctctggtttctctcgctttagtaggagcattggtcagcatcacaccgcaacaaagtcatgccgccttttttacaaccgtgatcattccagccattgttggagg

SEQ ID NO: 182 - DNA, 105 bp, Artificial Sequence
  Helicobacter pylori vacA nucleic acid sequence
ccctttggtttctctcgctttagtaggagcattggtcagcatcacaccgcaacaaagtcatgccgcctttttcacgaccgtgatcattccagccattgttggggg

SEQ ID NO: 183 - DNA, 105 bp, Artificial Sequence
  Helicobacter pylori vacA nucleic acid sequence
ccctctggtttctcttgctttagtaggagcgttagtcagcatcacaccgcaacaaagtcatgccgcctttttcacaaccgtgatcattccagccattgttggggg

SEQ ID NO: 184 - DNA, 105 bp, Artificial Sequence
  Helicobacter pylori vacA nucleic acid sequence
ccctctagtttctctcgctttagtaggagcattggtcagcatcacaccgcaacaaagtcatgccgcctttttcacaaccgtgatcattccagccattgtgggggg

SEQ ID NO: 185 - DNA, 105 bp, Artificial Sequence
  Helicobacter pylori vacA nucleic acid sequence
ccctttagtttctcttgctttagtaggagcattggtcagcatcacaccgcaacaaagtcatgccgcctttttcacaaccgtgatcattccagccattgttggggg

SEQ ID NO: 186 - DNA, 105 bp, Artificial Sequence
  Helicobacter pylori vacA nucleic acid sequence
ccctctngtttctctcgctttagtagnagcattngtcagcatcacaccgcaacanagtcatgccgcctttttcacaaccgtnatcattccagccattgttggggg

SEQ ID NO: 187 - DNA, 105 bp, Artificial Sequence
  Helicobacter pylori vacA nucleic acid sequence
ccctctggtttctctcgctttagtaggagcgttggtcagcatcacaccgcaacaaagtcatgccgcctttattacaaccgtgatcattccagccattgttggggg

SEQ ID NO: 188 - DNA, 105 bp, Artificial Sequence
  Helicobacter pylori vacA nucleic acid sequence
ccctctggtttctcttgctttagtaggagcattggtcagcatcacaccgcaacaaagtcatgccgccttttttacaaccgtgattattccagccattgtgggggg

SEQ ID NO: 189 - DNA, 105 bp, Artificial Sequence
  Helicobacter pylori vacA nucleic acid sequence
ccctctggtttctcttgctttagtaggagcattggtcagcatcacaccgcaacaaagtcatgccgctctttttacaaccgtgattattccagccattgtgggggg

SEQ ID NO: 190 - DNA, 105 bp, Artificial Sequence; identical to SEQ ID NO: 189
  Helicobacter pylori vacA nucleic acid sequence

SEQ ID NO: 191 - DNA, 105 bp, Artificial Sequence
  Helicobacter pylori vacA nucleic acid sequence
ccctctggtttctctcgctttagtaggagcattggtcagcatcacaccgcaacaaagtcatgccgctctttttacaaccgtgattattccagccattgtgggggg

SEQ ID NO: 192 - DNA, 105 bp, Artificial Sequence
  Helicobacter pylori vacA nucleic acid sequence
ccctytggtttctcttgctttagtaggagcattgrttagyrycayaccgcaacaaagtcatgccgcctttttyacraccgtgatcattccagccattgttggrgg

SEQ ID NO: 193 - DNA, 105 bp, Artificial Sequence
  Helicobacter pylori vacA nucleic acid sequence
ccctattatctctctcgctttagtaggagcattggtcagcatcacaccgcaacaaagtcatgccgcctttttcacaaccgtgttcattccagccattgttggggg

SEQ ID NO: 194 - DNA, 362 bp, Artificial Sequence
  Helicobacter pylori vacA nucleic acid sequence
gtggatgctcatacagcttattttaatggcaatatttatctgggaaaatccacgaatttaagagtgaatggccatagcgctcattttaaaaatattgatgccacaaagagcgataacgggctaaacactagcactttggattttagcggcgttacagacaaagtcaatatcaacaagctcactacatctgccactaatgtgaacattaaaaactttgacattaaggaattagtggttacgacccgtgttcagagttttgggcaatacactatttttggcgaaaatataggcgataagtctcgcattggtgtcgttagtttgcaaactggctatagcccggcctattctgggggcgttacttt

SEQ ID NO: 195 - DNA, 362 bp, Artificial Sequence
  Helicobacter pylori vacA nucleic acid sequence
gtggatgctcatacagcttattttaatggcaatatttatctgggaaaatccacgaatttaagagtgaatggccatagcgctcattttaaaaatattgatgccacaaagagcgataacgggctaaacactagtgctttggatttgagcggcgttacagacaaagtcaatatcaacaagctcactacatctgccactaatgtgaacattaaaaactttgacattaaggaattagtggttacaacccgtgttcagagttttgggcaatacactatttttggcgaaaatataggcgataagtcgcacattggtgtcgttagtttgcaaacgggatatagcccggcctattctgggggcgttacttt

SEQ ID NO: 196 - DNA, 362 bp, Artificial Sequence
  Helicobacter pylori vacA nucleic acid sequence
gtggatgctcatacagcttattttaatggcaatatttatctgggaaaatccacgaatttaagagtgaatggccatagcgctcattttaaaaatattgatgccacaaagagcgataacgggctaaacactagcgctttggatttgagcggcgttacagacaaagtcaatatcaacaagctcactacatctgccactaatgtgaacgttaaaaactttgacattaaggaattggtggttacaacccgagttcaaagttttgggcaatacactatttttggcgaaaatataggcgataagtcgcgcattggtgtcgttagtttgcaaacgggatatagcccggcctattctgggggcgttacttt

SEQ ID NO: 197 - DNA, 362 bp, Artificial Sequence; identical to SEQ ID NO: 196
  Helicobacter pylori vacA nucleic acid sequence

SEQ ID NO: 198 - DNA, 362 bp, Artificial Sequence
  Helicobacter pylori vacA nucleic acid sequence
gtggatgctcatacagcttattttaatggtaatatttatctgggaaaatccacgaatttaagagtgaatggccatagcgctcattttaaaaatattgatgccacaaagagcgataacgggctaaacactagcgctttggatttcagcggcgttacagataaagtcaatatcaacaagctcactacatctgccactaacgtgaacattaaaaactttgacattaaggaattggtggttacaacccgagttcaaagttttgggcaatacactatttttggcgaaaatataggcgataagtctcgcattggtgtcgtgagtttgcaaacgggatatagcccggcctattctgggggcgttacttt

SEQ ID NO: 199 - DNA, 362 bp, Artificial Sequence
  Helicobacter pylori vacA nucleic acid sequence
gtggatgctcatacagcttattttaatggcaatatttatctgggaaaatccacgaatttaagagtgaatggccatagcgctcattttaaaaatattgatgccagtaagagcgataacgggctaaacactagcactttggattttagcggcgttacagacaaagtcaatatcaacaagctcactacatctgccactaatgtgaacgttaaaaactttgacattaaggaattggtggttacaacccgagttcaaagttttgggcaatacactatttttggcgaaaatataggcgatcagtctcgcattggtgtcgtgagtttgcaaacgggatatagcccggcttattctgggggcgttacttt

SEQ ID NO: 200 - DNA, 362 bp, Artificial Sequence
  Helicobacter pylori vacA nucleic acid sequence
gtggatgctcatacagcttattttaatggcaatatttatctgggaaaatccacgaatttaagagtgaatggccatagcgctcattttaaaaatattgatgctagtaagagcgataacgggctaaacactagcgctttggattttagcggcgttacagacaaagtcaatatcaacaagctcactacatctgccactaatgtgaacattaaaaactttgacattaaggaattggtggttacaacccgagttcaaagttttgggcaatacactatttttggcgaaaatataggcgataagtctcgcattggtgtcgtgagtttgcaaacgggatatagcccggcctattctgggggcgttacttt

SEQ ID NO: 201 - DNA, 2 bp, Artificial Sequence
  Helicobacter pylori vacA nucleic acid sequence
wc

SEQ ID NO: 202 - DNA, 362 bp, Artificial Sequence
  Helicobacter pylori vacA nucleic acid sequence
gtggatgctcatacagcttattttaatggcaatgtttatctgggaaaatccacgaatttaagagtgaatggccatagcgctcattttaaaaatattgatgccagcaagagcgataacgggctaaacactagtgctttggattttagcggcgttacagacaaagtcaatatcaacaagctcactacatctgccactaatgtgaacattaaaaactttgacattaaggaattagtggttacaacccgagttcaaagttttgggcaatacactatttttggcgaaaatataggcgataagtctcgcattggtgtcgtgagtttgcaaacgggatatagcccggcctattctgggggcgttacttt

SEQ ID NO: 203 - DNA, 362 bp, Artificial Sequence; identical to SEQ ID NO: 202
  Helicobacter pylori vacA nucleic acid sequence

SEQ ID NO: 204 - DNA, 362 bp, Artificial Sequence; identical to SEQ ID NO: 202
  Helicobacter pylori vacA nucleic acid sequence

SEQ ID NO: 205 - DNA, 362 bp, Artificial Sequence
  Helicobacter pylori vacA nucleic acid sequence
gtggatgctcatacagcttattttaatggcaatgtttatctgggaaaatccacgaatttaagagtgaatgcccatagcgctcattttaaaaatattgatgccagcaagagcgataacgggctaaacactagtgctttggattttagcggcgttacagacaaagtcaatatcaacaagctcactacatctgccactaatgtgaacattaaaaactttgacattaaggaattagtggttacaacccgagttcaaagttttgggcaatacactatttttggcgaaaatataggcgataagtctcgcattggtgtcgtgagtttgcaaacgggatatagcccggcctattctgggggcgttacttt

SEQ ID NO: 206 - DNA, 362 bp, Artificial Sequence
  Helicobacter pylori vacA nucleic acid sequence
gtggatgctcatacagcttattttaatggcaatatttatctgggaaaatccacgaatttaagagtgaatggccatagcgctcattttaaaaatattgatgccacaaagagcgataacgggctaaacactagcgctttggattttagtggcgttacagacaaagtcaatatcaacaagctcactacatctgccactaatgtgaacattaaaaactttgacattaaggaattagtggttacaacccgagttcaaagttttgggcaatacactatttttggcgaaaatataggcgataagtctcgcattggtgtcgttagtttgcaaacgggatatagcccagcctattctgggggcgttacttt

SEQ ID NO: 207 - DNA, 362 bp, Artificial Sequence
  Helicobacter pylori vacA nucleic acid sequence
gtggatgctcatacagcttattttaatggcaatatttatctgggaaaatccacgaatttaagagtgaatggccatagcgctcattttaaaaatattgatgccagtaagagcgataacgggctaaacactagtgctttggattttagcggcgttacagacaaagtcaatatcaacaagctcactacatctgccactaatgtgaacattaaaaactttgacattaaggaattagttgttacaacccgagttcaaagttttgggcaatacactatttttggcgaaaatataggcgataagtctcgcattggtgtcgttagtttgcaaacgggatatagcccagcctattctgggggcgttacttt

SEQ ID NO: 208 - DNA, 362 bp, Artificial Sequence
  Helicobacter pylori vacA nucleic acid sequence
gtggatgctcatacagcttattttaatggcaatatttatctgggaaaatccacgaatttaaaagtgaatggccatagcgctcattttaaaaatattgatgccagtaagagcgataatggtctaaacactagcgctttggattttagcggcgttacagacaaagtcaatatcaacaagctcactacatctgccactaatgtgaacattaaaaactttgacattaaggaattagtggttacaacccgtgttcagagttttgggcaatacactatttttggcgaaaatataggcgataagtctcgcattggtgtcgttagtttgcaaatgggatatagcccggcctattctgggggcgttacttt

SEQ ID NO: 209 - DNA, 362 bp, Artificial Sequence
  Helicobacter pylori vacA nucleic acid sequence
gtggatgctcatacagcttattttaatggcaatatttatctgggaaaatccacgaatttaaaagtgaatggccatagcgctcattttaaaaatattgatgccagtaagagcgataacgggctaaacactagcgctttggatttgagcggcgttacagacaaagtcaatatcaacaagctcactacagctgccactaatgtgaacattaaaaactttgacattaaggaattggttgttacgacccgtgttcagagttttggacaatacactatttttggcgaaaatataggcgataagtctcgcattggtgtcgttagtttgcaaacgggatatagcccggcttattctgggggcgttacttt

SEQ ID NO: 210 - DNA, 362 bp, Artificial Sequence
  Helicobacter pylori vacA nucleic acid sequence
gtggatgctcatacagctaactttaatggcaatatttatttgggaaaatccacgaatttaagggtgaatggccatagcgctcattttaaaaatattgatgccagtaagagcgataacgggctaaacactagctctttggatttcagtggcgttacagacaaagtcaatatcaacaagctcactacatctgccactaatgtgaacgttaaaaactttgacattaaggaattggtggttacaacccgagttcagagttttgggcaatacactatttttggcgaaattataggcgataagtctcgcattggtgtcgttagtttgcaaacgggatatagcccggcctattctgggggcgttacttt

SEQ ID NO: 211 - DNA, 362 bp, Artificial Sequence
  Helicobacter pylori vacA nucleic acid sequence
gtggatgctcatacagcttattttaatggcaatatttatttgggaaaatccacgaatttaagagtgaatggccatagcgctcattttaaaaatattgatgccagtaagagcgataacgggctaaacactagtgctttggattttagcggcgttacagataaagtcaatatcaacaagctcactacatctgccactaatgtgaacgttaaaaactttgacattaaggaattggtggttacaacccgagttcaaagttttgggcaatacactatttttggcgaaaatataggcgataagtctcgcattggtgtcgttagtttgcaaacgggatatagcccggcctattctgggggcgttacttt

SEQ ID NO: 212 - DNA, 362 bp, Artificial Sequence
  Helicobacter pylori vacA nucleic acid sequence
gtggatgcccatacggctaactttaatggcaatatttatctgggaaaatccacgaatttaaaagtgaatggccatagcgctcattttaaaaatattgatgccacaaagagcgataacgggctaaacactagcgctttggatttgagtggcgttacagacaaagtcaatatcaacaagctcactacatctgccactaatgtgaacattaaaaactttgacattaaggaattagtggttacaacccgtgttcagagttttgggcaatacactatttttggcgaaaatataggcgataagtctcgcattggtgtcgttagtttgcaaacgggatatagcccggcctattctgggggcgttacttt

SEQ ID NO: 213 - DNA, 362 bp, Artificial Sequence
  Helicobacter pylori vacA nucleic acid sequence
gtggatgctcatacagcttattttaatggcaatatttatctgggaaaatccacgaatttaaaagtgaatggccatagcgctcattttaaaaatattgatgccagtaagagcgataatggtctaaacactagtgctttggatttgagcggcgttacagacaaagtcaatatcaacaagctcactacagctgccactaatgtgaacattaaaaactttgacattaaggaattagtggttacgacccgtgttcagagttttgggcaatacactatttttggcgaaaatataggagatcaatcgcgcattggtgtcgttagtttgcaaactggctatagcccggcctattctgggggcgttacttt

SEQ ID NO: 214 - DNA, 362 bp, Artificial Sequence
  Helicobacter pylori vacA nucleic acid sequence
gtggatgctcatacagcttattttaatggcaatatttatctgggaaaatccacgaatttaaaagtgaatggccatagcgctcattttaaaaatattgatgccactaagagcgataatggtctaaacactagcgctttggatttgagcggcgttacaaacaaggtcaatatcaacaagctcactacagctgccactaatgtgtccattaaaaactttgacattaaggaattagtggttacgacccgtgttcagagttttgggcaatacactatttttggcgaaaatataggcgatcaatcgcgcattggtgtcgttagtttgcaaactggctatagcccggcctattctgggggcgttacttt

SEQ ID NO: 215 - DNA, 362 bp, Artificial Sequence
  Helicobacter pylori vacA nucleic acid sequence
gtggatgctcatacggctaactttaatggcaatgtttatctgggaaaatccacgaatttaagagtgaatggccatagcgctcattttaaaaatattgatgccagtaagagcgataacgggctaaacactagcgctttggattttagcggcgttacagacaaagtcaatatcaacaagctcactacatctgccactaatgtgaacattaaaaactttgacattaaggaattggtggttacaacccgtgttcagagttttgggcaatacactatttttggcgaaaatataggcgataagtctcgcattggtgtcgttagtttgcaaacgggatatagcccggcctattctgggggcgttacttt

SEQ ID NO: 216 - DNA, 362 bp, Artificial Sequence
  Helicobacter pylori vacA nucleic acid sequence
gtggatgctcatacagcttattttaatggcaatgtttatctgggaaaatccacgaatttaagagtgaatggccatagcgctcattttaaaaatattgatgccagcaagagcgataacgggctaaacactagcgctttggattttagcggcgttacagataaagtcaatatcaacaagctcactacatctgccactaatgtgaacattaaaaactttgacattaaggaattagtggttacaacccgtgttcagagttttgggcaatacactatttttggcgaaaatataggcgataagtctcgcattggtgtcgtgagtttgcaaacgggatatagcccggcctattctgggggcgttacttt

SEQ ID NO: 217 - DNA, 362 bp, Artificial Sequence
  Helicobacter pylori vacA nucleic acid sequence
gtggatgctcatacagcttattttaatggcaatgtttatctgggaaaatccacgaatttaagagtgaatggccatagcgctcattttaaaaatattgatgctagtaagagcgataacgggctaaacactagcgctttggattttagcggcgttacagacaaagtcaatatcaacaagctcactacatctgccactaatgtgaacgttaaaaactttgacattaaggaattagtggttacaacccgtgttcagagttttgggcaatacactatttttggcgaaaatataggcgataagtctcgcattggtgtcgtgagtttgcaaacgggatatagcccagcctattctgggggcgttacttt

SEQ ID NO: 218 - DNA, 362 bp, Artificial Sequence
  Helicobacter pylori vacA nucleic acid sequence
gtggacgctcatacagcttattttaatggcaatatttatctgggaaaatccacgaatttaagagtgaatggccatagcgctcattttaaaaatattgatgccagcaagagcgataacgggctaaacactagcgctttggatttcagcggcgttacagacaaagtcaatatcaacaagctcactacatctgccactaatgtgaacgttaaaaactttgacgttaaggaattggtggttacaacccgtgttcagagttttgggcaatacactatttttggcgaaaatataggcgataagtctcgcattggtgtcgtgagtttgcaaacgggatatagcccggcctattctgggggcgttacttt

SEQ ID NO: 219 - DNA, 362 bp, Artificial Sequence
  Helicobacter pylori vacA nucleic acid sequence
gtggatgctcatacagcttattttaatggcaatatttatctgggaaaatccacgaatttaagagtgaatggccatagcgctcattttaaaaatattgatgccagcaagagcgataacgggctaaacactagcactttggattttagcggcgttacagataaagtcaatatcaacaagctcactacagctgccactaatgtgaacgttaaaaactttgacattaaggaattagtggttacaacccgagttcaaagttttgggcaatacactatttttggcgaaaatataggcgataagtctcgcattggtgtcgttagtttgcaaacgggatatagcccggcctattctgggggcgttacttt

SEQ ID NO: 220 - DNA, 362 bp, Artificial Sequence
  Helicobacter pylori vacA nucleic acid sequence
gtggatgctcatacagcttattttaatggcaatatttatttgggaaaatccacgaatttaagagtgaatggccatagcgctcattttaaaaatattgatgccagtaagagcgataacgggctaaacactagcgctttggattttagcggcgttacagacaaagtcaatatcaacaagctcactacatctgccactaatgtgaacgttaaaaactttgacattaaggaattagtggttacaacccgtgttcagagttttgggcaatacactatttttggcgaaaatataggcgataagtctcgcattggtgtcgttagtttgcaaacgggatatagcccggcctattctgggggcgttacttt

SEQ ID NO: 221 - DNA, 362 bp, Artificial Sequence; identical to SEQ ID NO: 211
  Helicobacter pylori vacA nucleic acid sequence

SEQ ID NO: 222 - DNA, 362 bp, Artificial Sequence
  Helicobacter pylori vacA nucleic acid sequence
gtggatgctcatacagcttattttaatggcaatatttatctgggaaaatccacgaatttaagattgaatggccatagcgctcattttaaaaatattgatgccagtaagagcgataacgggctaaacactagcgctttggattttagcggcgttacagacaaagtcaatatcaacaagctcactacatctgccactaatgtgaacgttaaaaactttgacattaaggaattggtggttacaacccgagttcagagttttgggcaatactctatttttggcgaaaatataggcgataagtcgcgcattggtgtcgttagtttgcaaacgggatatagcccggcctattctgggggcgttacttt

SEQ ID NO: 223 - DNA, 362 bp, Artificial Sequence
  Helicobacter pylori vacA nucleic acid sequence
gtggacgctcatacagcttattttaatggcaatatttatttgggaaaatccacgaatttaagagtgaatggccatagcgctcattttaaaaatattgatgccacaaagagcgataacgggctaaacactagcactttggatttgagcggcgttacagacaaagtcaatatcaacaagctcactacatctgccactaatgtgaacattaaaaactttgacattaaggaattagtggttacgacccgtgttcagagttttgggcaatacactatttttggcgaaaatataggcgataagtctcgcattggtgtcgtgagtttgcaaacgggatatagcccggcctattctgggggcgttacttt

SEQ ID NO: 224 - DNA, 362 bp, Artificial Sequence
  Helicobacter pylori vacA nucleic acid sequence
gtggatgctcatacagcttattttaatggcaatatttatctgggaaaatccacgaatttaagagtgaatggccatagcgctcattttaaaaatattgatgccacaaagagcgataacgggctaaacattagcactttggattttagcggcgttacagacaaagtcaatatcaacaagctcactacatctgccactaatgtgaacattaaaaactttgacattaaggaattagtggttacaacccgtgttcagagttttgggcaatacactatttttggcgaaaatataggcgataagtctcgcattggtgtcgttagtttgcaaacgggatatagcccggcctattctgggggcgttacttt

SEQ ID NO: 225 - DNA, 362 bp, Artificial Sequence
  Helicobacter pylori vacA nucleic acid sequence
gtggatgctcatacagcttattttaatggcaatatttatctgggaaaatccacgaatttgagagtgaatggccataacgctcattttaaaaatattgatgccagtaagagcgataacgggctaaacactagcactttggatttgagcggcgttacagacaaagtcaatatcaacaagctcactacagctgccactaatgtgaacattaaaaactttgacattaaggaattagtggttacgacccgtgttcagagttttgggcaatacactatttttggcgaaaatataggtgataagtctcgcattggtgtcgttagtttgcaaacgggatatagcccggcctattctgggggcgttacttt

SEQ ID NO: 226 - DNA, 362 bp, Artificial Sequence
  Helicobacter pylori vacA nucleic acid sequence
gtggatgctcatacagcttattttaatggcaatgtttatttgggaaaatccacgaatttaagagtgaatggccatagcgctcattttaaaaatattgatgccagtgagagcgataacgggctaaacactagcgctttggattttagcggcgttacagacaaagtcaatatcaacaagctcactacatctgccactaatgtgaacgttaaaaactttgacattaaggaattggtggttacgacccgtgttcagagttttgggcaatacactatttttggcgaaaatataggcgataagtctcgcattggtgtcgttagtttgcaaacgggatgtcgcccggcctgttctgggggcgttacttt

SEQ ID NO: 227 - DNA, 362 bp, Artificial Sequence
  Helicobacter pylori vacA nucleic acid sequence
ggggatgctcatacagcttattttaatggcaatatttatttgggaaaatccacgaatttaagagtgaatggccatagcgctcattttaaaaatattgatgccagtaagagcgataacgggctaaacactagcgytttggattttagcggcgttacagayaaagtcaatatcaacaagctcactacatctgccactaatgtgaacrttaaaaactttgayattaaggaattggtggttacaacccgagttcaaagttttgggcaatacactatttttggcgaaaatataggcgatmagtctcgcattggtgtcgttagtttgcaaacgggatatagcccrgcctattctgggggcgttacttt

SEQ ID NO: 228 - DNA, 362 bp, Artificial Sequence
  Helicobacter pylori vacA nucleic acid sequence
gtggatgctcatacagcttattttaatggcaatatttatctgggaaaatccacgaatttaagagtgaatggccatagcgctcattttaaaaatattgatgccacaaagagcgataatggtataaacactagcactttggatttgagcggcgttacagacaaggtcaatatcaacaagctcattacagcttccactaatgtgaacattaaaaactttgacattaaggaattggtggttacaacccgtgttcaaagttttgggcaatacactatttttggcgaaaatataggcgataagtctcgcattggtgtcgttagtttgcaaacgggatatagcccggcctattctgggggcgttacttt

SEQ ID NO: 229 - DNA, 362 bp, Artificial Sequence
  Helicobacter pylori vacA nucleic acid sequence
gtggatgctcatacggctaactttaatggcaatatttatctgggaaaatccacgaatttaagagtgaatggccatagcgctcatttttaaaatattgatgccagcaagagcgataacgggctaaacactagcaccttggatttcagtggcgttacagacaaagtcaatatcaacaagctcactacatctgccactaatgtgaacgttaaaaactttgatattaaggaattggtggttacaacccgagttcagagttttgggcaatacactatttttggcgaaaatataggcgataagtcgcgcattggtgtcgtgagtttgcaaacgggatatagcccagcttattctgggggcgttacttt

SEQ ID NO: 230 - DNA, 362 bp, Artificial Sequence
  Helicobacter pylori vacA nucleic acid sequence
gtggatgctcatacggctaactttaatggcaatatttatttgggaaaatccacgaatttgagagtgaatggccatagcgctcattttaaaaatattgatgccagtaagagcgataacgggctaaacactagcgctttggattttagcggcgttacagacaaagttaatatcaacaagctcactacatctgccactaatgtgaacgttaaaaactttgacattaaggaattggtggttacaacccgagttcaaagttttgggcaatacactatttttggcgaaaatataggcgataagtctcgcattggtgtcgtgagtttgcaaacgggatatagccctgcttattctgggggcgttacttt

SEQ ID NO: 231 - DNA, 362 bp, Artificial Sequence
  Helicobacter pylori vacA nucleic acid sequence
gtggatgctcatacagcttattttaatggcaatatttatctgggaaaatccacgaatttaagagtgaatggccatagcgctcattttaaaaatattgatgccagtaagagcgataacgggctaaacactaccactttggatttcagcggcgttacagataaagtcaatatcaacaagctcactacatctgccactaatgtgaacattaaaaactttgacattaaggaattagtggttacaacccgagttcagagttttgggcaatacactatttttggcgaaaatataggcgataagctgcacattggtgtcgtgagtttgcaaacgggatatagcccagcctattctggggggcttacttt

SEQ ID NO: 232 - DNA, 362 bp, Artificial Sequence
  Helicobacter pylori vacA nucleic acid sequence
gtggatgctcatacagcttattttaatggcaatatttatctgggaaaatccacgaatttaaaagtgaatggccatagcgctcattttaaaaatattgatgctagtaagagcgataacgggctaaacactagcgctttggatttgagcggcgttacaaacaaggtcaatatcaacaagctcactacagctgccactaatgtgaacattaaaaactttgacattaaggaattggtggttacaacccgcgttcagagttttgggcaatacactatttttggcgaaaatataggcgataagtcgcgcattggtgtcgttagtttgcaaactggctatagcccggcctattctgggggcgttacttt

SEQ ID NO: 233 - DNA, 362 bp, Artificial Sequence
  Helicobacter pylori vacA nucleic acid sequence
atggatgctcatacagcttattttaatggcaatatttatctgggaaaatccacgaatttaaaagtgaatggccatagcgctcattttaaaaatattgatgccactaagagcgataatggtctaaacactagcgctttggatttgagcggcgttacaaacaaggtcaatatcaacaagctcactacagctgccactaatgtgtccattaaaaactttgacattaaggaattagtggttacgacccgtgttcagagttttgggcaatacactatttttggcgaaaatataggcgatcaatcgcgcattggtgtcgttagtttgcaaactggctatagcccggcctattctgggggcgttacttt

SEQ ID NO: 234 - DNA, 362 bp, Artificial Sequence
  Helicobacter pylori vacA nucleic acid sequence
gtggatgctcatacagcttattttaatggcaatatttatctgggaaaatccacgaatttaaaagtgaatggccatagcgctcattttaaaaatattgatgccactaagagcgataatggtctatacactagcgctttggatttgagcggcgttacaaacaaggtcaatattaacacgctcactacagctgccactaatgtgtccattaaaaactttgacattaaggaattagtggttacgacccgtgttcagagttttgggcaatacactatttttggcgaaaatataggcgatcaatcgcgcattggtgtcgttagtttgcaaactggctatagcccggcctattctgggggcgttacttt

SEQ ID NO: 235 - DNA, 362 bp, Artificial Sequence
  Helicobacter pylori vacA nucleic acid sequence
gtggatgcccatacgatcaattttaatggcaatatgtatttgggaagatttacgcatttaaaagtgaatggtcatacagccaattttaaagatattgatgccagcaagggtagaaatggtatcgacaccaccattttggattttagcggcgttacaaacaaggtcaatatcaacaagctcaccacagctgccactaatgcggccattaaaaattttgacattaaggaattggttgttacaaccaatgttttgagtgtggggaaatacactgattttaccgaagatataggcgatcaatcccgcattggtatcgtgcgtttgcaaatgggatatagcccggcctattctgggggcgttacttt

SEQ ID NO: 236 - DNA, 362 bp, Artificial Sequence
  Helicobacter pylori vacA nucleic acid sequence
gtggatgcccatacgatcaattttaatggcaatatgtatttgggaagattcacgcatttaaaagtgaatggtcatacagccaattttaaagatattgatgccagcaagggtagaaatggtatcgacaccaccattttggattttagcggcgttacaaacaaggtcaatatcaacaagctcaccacagctgccactaatgcggccattaaaaattttgacattaaggaattggttgttacaaccaatgttttgagtgtggggaaatacactgattttaccgaagatataggcgatcaatcccgcattggtatcgttagtttgcaaacgggatatagcccggcctattctgggggcgttacttt

SEQ ID NO: 237 - DNA, 362 bp, Artificial Sequence
  Helicobacter pylori vacA nucleic acid sequence
gtggatgcccatacgatcaattttaatggcaacatgtatttgggaagattcacgcatttaaaagtgaatggccatacagccaattttaaagatattgatgccagcaagggtagaaatggtatcgacaccactattttggattttagcggcgttacagacaaagtcaatatcaacaagctcactacagctgccactaatgtgtccattaaaaactttgacattaaggaattggttgttacaaccaatgttttgagtgtggggaaatacactgattttaccgaagatataggcgatcaatcgcacattggtgtcgttagtttgcaaactggctatagcccggtctattctgggggcgttacttt

SEQ ID NO: 238 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacaggtaaggtcaatatcaacaagctcattacggcttccactaatgtggccgctaaaaacttcaacattaatgaattgattgttaaaaccaatggggtgagtgtgggggaatacactcattttagcgaagatataggcagtcaatcgcgcatcaataccgtgcgtttggaaactggcactaggtcaatctattctggcggtgttaaattt

SEQ ID NO: 239 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacaggtaaggtcaatatcaacaagctcatcacagcttccactaatgtggccgttaaaaacttcaacattaatgaattgattgttaaaaccaatggtgtgagtgtgggggaatacactcattttagcgaagatataggcagtcaatcgcgcatcaataccgtgcgtttggaaactggcactaggtcaatcttttctgggggtgttaaattt

SEQ ID NO: 240 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacaggtaaggtcaatatcaacaagctcatcacagcttccactaatgtggccgttaaaaacttcaacattaatgaattgattgttaaaaccaatgggataagtgtgggggaatacactcattttagcgaagatataggaagtcaatcgcgcatcaataccgtgcgtttggaaactggcactagatcaatcttttctgggggtgttaaattt

SEQ ID NO: 241 - DNA, 288 bp, Artificial Sequence
  Helicobacter pylori vacA nucleic acid sequence
gtggatgctcatacagctaattttaaaggtattgatacgggcaatggtggtttcaacaccttagattttagtggcgttacagacaaggtcaatatcaacaagctcattacagcttccactaatgtggccattaaaaacttcaacattaatgaattgttggttaagaccaatggggtgagcgtgggggaatacactcattttagcgaagatataggcagtcaatcgcgcatcaataccgtgcgtttggaaactggcactaggtcaatcttttctgggggtgtcaaattt

SEQ ID NO: 242 - DNA, 288 bp, Artificial Sequence
  Helicobacter pylori vacA nucleic acid sequence
gtggatgctcatacagctaattttaaaggtattgatacgggtaatggtggtttcaacaccttagattttagtggcgttacagacaaggtcaatatcaacaagctcattacagcttccactaatgtggccattaaaaacttcaacattaatgaattggttgttaagaccaatggggtgagtgtgggggaatacactcattttagcgaagatataggcagtcaatcgcgcatcaacaccgtgcgtttggaaactggcactaggtcaatctattctgggggtgttaaattt

SEQ ID NO: 243 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacaggtaaggtcaatatcaacaagctcatcacggcttccactaatgtggccgttaaaaacaacaacattaatgaattggtggttaaaaccaatgggataagtgtgggggaatacactcattttagcgaagatataggcagtcaatcgcgcatcaataccgtgcgtttggaaacaggcactaggtcaatcttttctgggggtgtcaaattt

SEQ ID NO: 244 - DNA, 288 bp, Artificial Sequence
  Helicobacter pylori vacA nucleic acid sequence
gtggatgctcatacagctaattttaaaggtattgatactggtaatggtggtttcaacaccttagattttagtggtgttacaaacaaagtcaatatcaacaagctcattacagcttccactaatgtggccgttaaaaacttcaacattaatgaattgttggttaagattaatggggtgagtgtgggggaatacacttattttagcgaagatataggcagtcaatcgcgcatcaacaccgtgcgtttggaaactggcactaggtcaatctattctggcggtgttaaattt

SEQ ID NO: 245 - DNA, 288 bp, Artificial Sequence
  Helicobacter pylori vacA nucleic acid sequence
gtggatgctcatacagctaattttaaaggtattgatactggtaatggtggtttcaacaccttagatttcagtggtgttacagacaaggtcaatatcaacaagctcattacagcttccactaatgtggccattaaaaacttcaacattaatgaattggtggttaaaaccaatggtataagcgtgggggaatacactcattttagcgaagatataggcagtcaatcgcgcatcaacaccgtgcgtttggaaactggcactaggtcaatctattctggcggtgttaaattt

SEQ ID NO: 246 - DNA, 288 bp, Artificial Sequence
  Helicobacter pylori vacA nucleic acid sequence
gtggatgctcatacagctaattttaaaggtattgatactggtaatggtggtttcaacaccttagatttcagtggtgttacagacaaggtcaatatcaacaagctcattacagcttccactaatgtggccattaaaaacttcaacattaatgaattggtggttaaaaccaatggtataagcgtgggggaatacactcattttagcgaagatataggcagtcaatcgcgcatcaataccgtgcgtttggaaactggcactaggtcaatctattctggcggtgttaaattt

SEQ ID NO: 247 - DNA, 288 bp, Artificial Sequence
  Helicobacter pylori vacA nucleic acid sequence
gtggatgcccatacagctaattttaaaggtattgatactggtaatggtggtttcaacaccttagatttcagtggcgttacaaacaaagtcaatatcaacaagctcattacagcttccactaatgtggccattaaaaacttcaacattaatgaattggtggttaaaaccaatggtataagcgtgggggaatacactaattttagcgaagatataggcagtcaatcgcgcatcaacaccgtgcgtttggaaactggcactaggtcaatctattctggcggtgttaaattt

SEQ ID NO: 248 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacaggtaaggtcaatatcaacaagctcatcacagcttccactaatgtggccgttaaaaacttcaacattaatgaattgattgttaaaaccaatggggtgagtgtgggggaatacactcattttagcgaagatataggcagtcaatcgcgcatcaataccgtgcgtttggaaactggcactaggtcaatctattctggcggtgttaaattt

SEQ ID NO: 249 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacaggtaaggtcaatatcaacaagctcatcacagcttccactaatgtggccgctaaaaacttcaacattaatgaattgattgttaaaaccaatggggtgagtgtgggggaatacactcattttagcgaagatataggcagtcaatcgcgcatcaataccgtgcgtttggaaactggcactaggtcaatctattctggcggtgttaaattt

SEQ ID NO: 250 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacaggtaaggtcaatatcaacaagctcatcacagcttccactaatgtggccgttaaaaacttcaacattaatgaattgattgttaaaaccaatggtgtgagtgtgggggaatacactcattttagcgaagatataggcagtcaatcgcgcatcaacaccgtgcgtttggaaactggcactaggtcaatctattctggcggtgttaaattt

SEQ ID NO: 251 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacaggtaaggtcaatattaacaagctcattacggcttccactaatgtggccgttaaaaacttcaacattaatgaattgattgttaaaaccaatggggtgagtgtgggggaatacactcattttagcgaagatataggcagtcaatcgcgcatcaataccgtgcgtttggaaactggcactaggtcaatcttttctgggggtgttaaattt

SEQ ID NO: 252 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgtgacaggtatagtcaatatcaacaagctcatcacagcttccactaatgtggccgttaaaaacttcaacattaatgaattgattgttaaaaccaatggggtgagcgtgggggaatacactcattttagcgaagatataggcagtcaatcgcgcatcaataccgtgcgtttggaaactggcactaggtcaatcttttctgggggtgtcaaattt

SEQ ID NO: 253 - DNA, 288 bp, Artificial Sequence
  Helicobacter pylori vacA nucleic acid sequence
gtggatggtcatacagctaattttaaaggtattgatacgggtaatggtggtttcaacaccttagattttagtggtgttacaggtaaggtcaatatcaacaagctcataacggcttccactaatgtggccgttaaaaacttcaacattaatgaattgattgttaaaaccaatggtgtgagtgtgggggaatacacttattttagcgaagatataggcagtcaatcgcacatcaataccgtgcgtttggaaactggcactaggtcaatcttttctgggggtgtcaaattt

SEQ ID NO: 254 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacaggtaaggtcaatatcaacaagctcattacggcttccactaatgtggccgttaaaaacttcaacattaatgaattgattgttaaaaccaatggtgtgagtgtgggggaatacacttattttagcgaagatataggcagtcaatcgcacatcaataccgtgcgtttggaaactggcactaggtcaatcttttctgggggtgtcaaattt

SEQ ID NO: 255 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacagacaaagtcaatatcaacaagctcatcacagcttccactaatgtggccgttaaaaacttcaacattaatgaattgattgttaaaaccaatggggtgagtgtgggggaatacactcattttagcgaagatataggcagtcaatcgcgcatcaataccgtgcgtttggaaactggcaccaggtcaatcttttctgggggtgtcaaattt

SEQ ID NO: 256 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacagacaaagtcaatatcaacaagctcattacggcttccactaatgtggccgttaaaaacttcaacattaatgaattgattgttaaaaccaatggggtgagtgtgggggaatacactcattttagcgaagatataggcagtcaatcgcgcatcaataccgtgcgtttggaaactggcactaggtcaatcttttctgggggtgtcaaattt

SEQ ID NO: 257 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacagacaaagtcaatatcaacaagctcattacagcttccactaatgtggccgttaaaaacttcaacattaatgaattggttgttaaaaccaatggggtaagtgtgggggaatacactcattttagcgaagatataggcagtcaatcgcacatcaataccgtgcgtttggaaactggcactaggtcaatcttttctgggggtgtcaaattt

SEQ ID NO: 258 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacaaacaaggtcaatatcaacaagctcattacggcttccactaatgtggccattaaaaacttcaacattaatgaattgattgttaaaaccaatgggatgagcgtgggggaatacactcattttagcgaagatataggcagtcaatcgcgcatcaataccgtgcgtttggaaactggcactaggtcaatcttttctgggggtgtcaaattt

SEQ ID NO: 259 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacaaacaaggtcaatatcaacaagctcattacagcttccactaatgtggccgttaaaaacttcaacattaatgaattgattgttaaaaccaatggggtgagcgtgggggaatacactcattttagcgaagatataggcagtcaatcgcgcatcaataccgtgcgtttggaaactggcactaggtcaatcttttctgggggtgtcaaattt

SEQ ID NO: 260 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacaaacaaggtcaatatcaacaagctcattacggcttccactaatgtggccgttaaaaacttcaacattaatgaattgattgttaaaaccaatggggtgagcgtgggggaatacactcattttagcgaagatataggcagtcaatcgcgcatcaataccgtgcgtttggaaactggcactaggtcaatcttttctgggggtgtcaaattt

SEQ ID NO: 261 - DNA, 288 bp, Artificial Sequence
  Helicobacter pylori vacA nucleic acid sequence
gtggatgctcatacagctaattttaaaggtattgatacgggtaatggtggtttcaacaccttggattttagtggcgttacagacaaagtcaatatcaacaagctcattacagcttccactaatgtggccattaaaaacttcaacattaatgaattgttggttaagaccaatggggtgagtgtgggggaatacactcattttagcgaagatataggcagtcaatcgcgcatcaacaccgtgcgtttagaaactggcactaggtcaatcttttctgggggtgtcaaattt

SEQ ID NO: 262 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacaggtaaggtcaatatcaacaagctcattacggcttccactaatgtggccgttaaaaacttcaacattaatgaattgttggttaagaccaatggggtgagtgtgggggaatacactcattttagcgaagatataggcagtcaatcgcgcatcaataccgtgcgtttggaaactggcactaggtcaatcttttctgggggtgtcaaattt

SEQ ID NO: 263 - DNA, 288 bp, Artificial Sequence
  Helicobacter pylori vacA nucleic acid sequence
gtggatgctcatacagctaattttaaaggtattgatacgggtaatggtggtttcaacaccttagattttagtggtgttacaggtaaggtcaatatcaacaagctcattacggcttccactaatgtagccgttaaaaacttcaacattaatgaattgttggttaagaccaatggggtgagtgtgggggaatacactcattttagcgaagatataggcagtcaatcgcgcatcaacaccgtgcgtttggaaactggcactaggtcaatcttttctgggggtgtcaaattt

SEQ ID NO: 264 - DNA, 288 bp, Artificial Sequence
  Helicobacter pylori vacA nucleic acid sequence
gtggatggtcatacagctaattttaaaggtattgatacgggtaatggtggtttccacaccttagattttagtggtgttacaggtaaggtccatatccacaagctcattacggcttccactaatgtggccgttaaaaacttccacattaatgaattgattggtaaaaccaatgggataagtgtgggggaatacactcattttagcgaagatataggcagtcaatcgcgcatcaataccgtgcgtttggaaactggcactaggtcaatcttttctgggggtgtcaaattt

SEQ ID NO: 265 - DNA, 288 bp, Artificial Sequence
  Helicobacter pylori vacA nucleic acid sequence
gcgagcgctcatacggtcaattttaaagatattgatactggtaatggtggtttcaacaccttagattttagtggtgttacaaacaaggtcaatatcaacaagctcattacagcttccactaatgtggccattaaaaacttcaacattaatgaattgttggttaaaaccaatgggataagtgtgggggaatacactaattttagcgaagatataggcaatcaatcgcgcatcaacaccgtgcgtttggaaactggcaccaggtcaatctattttgggggtgttaaatta

SEQ ID NO: 266 - DNA, 288 bp, Artificial Sequence
  Helicobacter pylori vacA nucleic acid sequence
gcgagcgctcatacggtcaattttaaagatattgatactggtaatggtggtttcaacaccttagactttagtggtgttacaaacaaggtcaatatcaacaagctcattacagcttccactaatgtggccgttaaaaacttcaacattaatgaattgttggttaaaaccaatgggataagtgtgggggaatacactaattttagcgaagatataggcaatcaatcgcgcatcaacaccgtgcgtttggaaactggcactaggtcaatctattctgggggtgttaaattt

SEQ ID NO: 267 - DNA, 288 bp, Artificial Sequence
  Helicobacter pylori vacA nucleic acid sequence
gcgagcgctcatacggtcaattttaaagatattgatactggtaatggtggtttcaacaccttagactttagtggtgttacaaacaaggtcaatatcaacaagctcattacagcttccactaatgtggccattaaaaacttcaacattaatgaattgttggttaaaaccaatgggataagtgtgggggaatacactaattttagcgaagatataggcaatcaatcgcacatcaacaccgtgcgtttagaaactggcactaggtcaatctattctgggggtgttaagttt

SEQ ID NO: 268 - DNA, 288 bp, Artificial Sequence
  Helicobacter pylori vacA nucleic acid sequence
gcgagacgtcatacggtcaattttaaagatattgatactggtaatggtggtttcaacaccttagattttagtggtgttacaaacaaggtcaatatcaacaagctcattacagcttccactaatgtggccgttaaaaacttcaacattagtgaattgttggttaaaaccaatgggataagtgtgggggaatacactaattttagcggagatataggcaatcaatcgcgcatcaacaccgtgcgtttggaaactggcactaggtcaatctattctgggggtgttaagttt

SEQ ID NO: 269 - DNA, 288 bp, Artificial Sequence
  Helicobacter pylori vacA nucleic acid sequence
gtggatgcccatacggtcaattttaaaggtattgatactggtaatggtggtttcaacaccttagatttcagtggtgttacagacaaggtcaatatcaacaagctcattacagcttccactaatgtggccattaaaaacttcaacattaatgaattggtggttaaaaccaatggtataagcgtgggggaatacactcattttagcgaagatataggcagtcaatcgcgcatcaataccgtgcgtttggaaactggcactaggtcaatctattctggcggtgttaaattt

SEQ ID NO: 270 - DNA, 288 bp, Artificial Sequence
  Helicobacter pylori vacA nucleic acid sequence
gcgagcgctcatacggtcaattttaaagatattgatactggtaatggtggtttcaacaccttagactttagtggtgttacaaacaaggtcaatatcaacaaactcattacagcttccactaatgtggccgttaaaaacttcaacattaatgaattgttggttaaaaccaatgggataagtgtgggggaatacactaattttagcgaagatataggcaatcaatcgcgcatcaacaccgtgcgtttagaaactggcaccaggtcaatctattctgggggtgttaagttt

SEQ ID NO: 271 - DNA, 288 bp, Artificial Sequence
  Helicobacter pylori vacA nucleic acid sequence
gcgagcgctcatacggtcaattttaaagatattgatactggtaatggtggtttcaacaccttagactttagtggtgttacaaacaaggtcaatatcaacaaactcattacagcttccactaatgtggccgttaaaaacttcaacattaatgaattgttggttaaaaccaatgggataagtgtgggggaatacactaattttagcgaagatataggcagtcaatcgcgcatcaataccgtgcgtttagaaactggcaccaggtcaatctattctgggggtgttaagttt

SEQ ID NO: 272 - DNA, 288 bp, Artificial Sequence
  Helicobacter pylori vacA nucleic acid sequence
gcgagcgctcatacggtcaattttaaagatattgatactggtaatggtggtttcaacaccttagattttagtggtgttacaaacaaggtcaatatcaacaagctcattacagcttccactaatgtggccattaaaaacttcaacattaatgaattgttggttaaaaccaatgggataagtgtgggggaatacactaattttagcgaagatataggcaatcaatcgcgcatcaataccgtgcgtttagaaactggcactaggtcaatctattctgggggtgttaagttt

SEQ ID NO: 273 - DNA, 288 bp, Artificial Sequence
  Helicobacter pylori vacA nucleic acid sequence
gcgagcgctcatacggtcaattttaaagatattgatactggtaatggtggtttcaacaccttagattttagtggtgttacaaacaaggtcaatatcaacaagctcattacagcttccactaatgtggccgttaaaaacttcaacattaatgaattgttggttaaaaccaatgggataagtgtgggggaatacactaattttagcgaagatataggcaatcaatcgcgcatcaacaccgtgcgtttggaaactggcactaggtcaatctattctgggggtgttaagttt

SEQ ID NO: 274 - DNA, 288 bp, Artificial Sequence
  Helicobacter pylori vacA nucleic acid sequence
gcgagcgctcatacggtcaattttaaagatattgatactggtaatggtggtttcaacaccttagactttagtggtgttacaaacaaggtcaatatcaacaagctcattacagcttccactaatgtggccgttaaaaacttcaacattaatgaattgttggttaaaaccaatgggataagtgtgggggaatacactaattttagcgaagatataggcaatcaatcgcgcatcaacaccgtgcgtttagaaactggcactaggtcaatctattctgggggtgttaaattt

SEQ ID NO: 275 - DNA, 288 bp, Artificial Sequence
  Helicobacter pylori vacA nucleic acid sequence
gcgagcgctcatacggtcaattttaaagatattgatactggtaatggtggtttcaataccttagactttagtggtgttacaaacaaggtcaatatcaacaagctcattacagcttccactaatgtggccattaaaaatttcaacattaatgaattgttggttaaaaccaatgggataagtgtgggggaatacactaattttagcgaagatataggcagtcaatcgcgcatcaacaccgtgcgtttagaaactggcactaggtcaatctattctgggggtgttaagttt

SEQ ID NO: 276 - DNA, 288 bp, Artificial Sequence
  Helicobacter pylori vacA nucleic acid sequence
acgagcgctcatacggtcaattttaaagatattgatactggtaatggtggtttcaacaccttagactttagtggtgttacaaacaaggtcaacatcaacaagctcattacagcttccactaatgtggccattaaaaacttcaacattaatgagttgttggttaaaaccaatgggataagtgtgggggaatacactaattttagcgaagatataggcaatcaatcgcgcatcaacaccgtgcgtttagaaactggcactaggtcaatctattctgggggtgttaagttt

SEQ ID NO: 277 - DNA, 21 bp, Artificial Sequence
  vacA primer
atggaaatacaacaaacacac

SEQ ID NO: 278 - DNA, 19 bp, Artificial Sequence
  vacA primer
ctgcttgaatgcgccaaac

SEQ ID NO: 279 - DNA, 21 bp, Artificial Sequence
cacagccactttcaataacga

SEQ ID NO: 280 - DNA, 20 bp, Artificial Sequence
  vacA primer
cgtcaaaataattccaaggg

(19) United States
(12) Patent Application Publication
(10) Pub. No.: US 2003/0165860 A1
(43) Pub. Date: Sep. 4, 2003

(54) Probes, methods and kits for detection and typing of Helicobacter pylori nucleic acids in biological samples

(76) Inventors: Wilhelmus Quint, Nootdorp (NL); Leendert-Jan Van Doorn, Ridderkerk (NL)

Correspondence Address: KNOBBE MARTENS OLSON & BEAR LLP, 2040 MAIN STREET, FOURTEENTH FLOOR, IRVINE, CA, 92614, US

(21) Appl. No.: 10/035,978

(22) Filed: Dec. 21, 2001

Related U.S. Application Data
(63) Continuation of application No. 09/284,725, filed on Apr. 16, 1999, now abandoned.
Application No. 09/284,725 is a 371 of international application No. PCT/EP97/05614, filed on Oct. 10, 1997.

(30) Foreign Application Priority Data
Oct. 16, 1996 (EP) 96870131.8
Sep. 9, 1997 (EP) 96870133.2

(51) Int. Cl. C12Q 1/68; C12P 19/34

(52) U.S. Cl. 435/6; 435/91.2

(57) ABSTRACT

The present invention relates to a method for the detection and/or typing of Helicobacter pylori (H. pylori) strains present in a sample comprising the steps of: (i) if need be releasing, isolating or concentrating the polynucleic acids in the sample, (ii) amplifying the polynucleic acids of relevant target regions of the vacA gene and possibly other virulence determinant genes (VDG), with suitable primer pairs, said primers being generally applicable on different H. pylori strains, allowing to amplify said relevant target regions of the VDG preferentially in compatible amplification conditions; (iii) hybridizing the polynucleic acids obtained in (i) or (ii) with a set of at least two VDG-derived probes, under appropriate hybridization and wash conditions, and with at least one of said probes hybridizing to a conserved region of a VDG of H. pylori, and with at least one of said probes hybridizing to a variable region of vacA; (iv) detecting the hybrids formed in step (iii), (v) detecting and/or typing H. pylori strains present in a sample from the differential hybridization signals obtained in step (iv), with said typing being the allele-specific detection of a strain according to the VDG alleles present in that particular H. pylori strain, and the said virulence determinant genes being the genetic elements involved in enabling, determining, and marking of the infectivity and/or pathogenicity of said H. pylori strain. The present invention also relates to probes and primers for doing the same as well as Helicobacter pylori detecting/typing kits. The present invention also discloses novel sequences of VDG, which can be used for designing the above-mentioned primers and probes.

This invention relates to the field of the detection and typing of the human pathogen Helicobacter pylori, abbreviated as H.pylori below.

This invention relates to probes, primers, methods, and kits comprising the same for the detection and typing of nucleic acids of H.pylori in biological samples.

H.pylori is the causative agent of chronic superficial gastritis in humans and infection with this organism is a significant risk factor for the development of peptic ulcer disease and gastric cancer. (Blaser et al. 1992; Hentschel et al. 1993; Parsonnet et al., 1991)

The outcome of an infection with H.pylori is rather diverse, probably reflecting the large diversity within the species at the genetic level (Foxall et al., 1992: Akopyanz et al., 1992). However, most phenotypic characteristics are well conserved. As individuals can be infected with various strains, it will however be important to identify particular characteristics of different H.pylori strains that precisely determine risk among these strains.

Among the respective virulence determinants of H.pylori, two important genetic elements have been identified recently the vacuolating toxin gene (vacA gene) and the cytotoxin associated gene (cagA gene) (Leunk et al 1988: Cover and Blaser, 1992, 1995; Cover et al. 1992, 1994, Tummuru et al., 1993; Covacci et al. 1993).

The H.pylori vacuolating toxin induces cytoplasmic vacuolation in a large number of mammalian cell lines in vitro (Leunk et al. 1988), and produces epithelial cell damage and mucosal ulceration when administrated intragastrically to mice(Telford et al., 1993). The vacA gene encodes a 1287-1296 amino acid precursor which is processed (N- and C-terminally) to a 87-Kda secreted protein (Cover and Blaser, 1992; Cover et al., 1994; Telford et al. 1994; Schmitt and Haas, 1994; Phadnis et al., 1994). Although only 50% of the H.pylori strains induce vacuolation, nearly all strains hybridize to vacA probes (Cover et al. 1994: Telford et al., 1994; Schmitt and Haas 1994; Phadnis et al. 1994). Very recently, Atherton et al. (1995) gave evidence for a mosaic organisation of the vacA gene, which indicated that specific vacA genotypes of H.pylori swains are associated with the level of cytotoxin activity, in vitro as well as with the clinical consequences.

It was shown that three different classes of vacA signal sequences (s1a, s2b and S2) are present and two different classes of middle-region alleles (m2 and m2). All possible combinations of these vacA regions have been isolated, with the exception of s2/m1. The production of cytotoxin activity was strongly linked to the presence of vacA alleles containing the s1-type signal peptide. None of the strains containing s2-type vacA alleles produced detectable cytotoxin activity. Also, a significant correlation between the occurrence of peptic ulceration and the presence of s1-type vacA alleles could be demonstrated

A second putative virulence determinant is the high molecular weight protein encoded by the cytotoxin-associated gene, cagA (Tummuru et al., 1993; Covacci et al., 1993). About 60% of the H pylori strains possess the cagA gene and nearly all of them express the cagA gene product Production of the vacuolating cytotoxin in vitro and the presence of cagA are closely associated characteristics, although both genes are not tightly genetically linked (Turn et al., 1993; Covacci et al, 1993).

Based on immumoblot studies, it has been demonstrated that persons infected with cagA(+)-strains have higher degrees of gastric inflammation and epithelial cell damage in comparison to infections with cagA(+)-strains Also, an inhanced expression of a number of cytokines has been found with respect to infection with cagA(+)-strains in comparison to cagA(−)-strains (Huang et al.,1995). As both the intensity of the inflammmation and the degree of epithelial damage may be determining the pathogenesis of gastric cancer, the examination of the presence or abscence of the cagA gene upon H.pylori infection is important.

In this invention, it is disclosed for the first time that the methods described by Atherton et al., 1995 are not suitable to type H.pylori strains present in a number of clinical samples obtained from patients of the Netherlands and Portugal (see example 1). Moreover, the typing method described by these authors involves the resolution of gene-amplification products by agarose gel electrophoresis, a tedious and not highly reliable technique when applied on large number of samples.

Thus, with respect to the nessecity to evaluate large populations to provide statistically relevant data concerning the linkage between a type of H.pylori strains and any pathogenic phenotype and in view of the need for a rapid, simple and highly reliable typing method in order to determine the applicable eradication strategy at the clinical stage, the above method described by Atherton et al, 1995 is less appropriate.

It is an aim of this present invention to provide a rapid, sensitive and reliable method to detect and type H. pylori strains in biological samples.

More particularly, it is an aim of the present invention to provide a rapid, sensitive and reliable method to detect and/or type H.pylori strains in biological samples, associated with the development of chronic active gastritis and/or gastric and duodenal ulcers, and/or gastric adenocarcinomas and/or mucosa-associated lymphoid tissue lymphomas, and/or to determine the applicable eradication therapy.

It is an aim of the present invention to provide a rapid, sensitive and reliable method to detect and type H.pylori strains present in biological samples, directly coupled to the detection and/or the typing of the alleles of the virulence determinant genes present, including at least the vacA gene.

More particularly, it is an aim of the present invention to provide a rapid, sensitive and reliable method to detect and type H. pylori strains present in a biological sample, directly coupled to the detection and/or the typing of the vacA and cagA alleles present.

It is the aim of the present invention to define suitable probes enabling the detection and/or allele-specific typing of H.pylori strains based on the alleles of the virulence determinant genes present, including at least one probe derived from vacA More particularly, it is an aim of the present invention to define suitable probes enabling the detection and/or allele-specific typing of H pylori strains based on the alleles of the vacA and cagA virulence determinant genes present.

It is moreover an aim of the present invention to combine the suitable probes enabling detection and/or allele-specific typing of H.pylori strains based on the alleles of the virulence determinant genes present, including at least the vacA gene, whereby all said probes can preferentially be used simultanously in a multiparameter type of assay, more particularly under the same hybridisation and wash-conditions.

More particularly, it is an aim of the present invention to combine the suitable probes enabling detection and/or allele-specific typing of H.pylori strains based on the alleles of the vacA and cagA genes present, whereby all probes can be preferentially used simultanously under the same hybridisation and wash-conditions.

More particularly, it is an aim of this invention to develop suitable probes of relevant target regions of the VDG, including at least the vacA gene, said target regions comprising either a variable region, either a conserved region of the VDG, said probes being applicable, if appropriate, in a simultanous hybridisation assay.

Even more particularly, it is an aim of this invention to develop suitable probes of relevant target regions of the vacA and cagA genes, said target regions comprising a variable region in case of the vacA gene and a conserved region in case of the cagA gene, said probes being applicable, if appropriate, in a simultanous hybridisation assay.

Most particularly, it is an aim of this invention to design suitable probes comprising the highly variable S- and M-regions in the vacA gene, said S-region being comprised between the nucleotides at position 1 and 300, and said M-regions being comprised between the nucleotides at the position 1450 and 1650, and a common probe in the case of the cagA gene comprising preferentially the highly conserved region between the nucleotide at the position 17 and the nucleotide at the position 113 of the cagA gene of H.pylori, if appropriate, in a simultanous hybridisation assay.

It is also an aim of the present invention to select primers enabling the amplification of relevant target regions of alleles of the virulence determinant gene of interest of H.pylori including at least the vacA gene, said amplification being universal for the respective target regions, said target regions comprising either a variable region or a conserved region of the VDG.

It is more particularly an aim of the present invention to select primers enabling the amplification of the relevant target regions of the alleles of the vacA and cagA virulence determinant genes of the H.pylori, said primers being being generally applicable with H.pylori strains and allowing the amplification of said relevant target regions to be used in compatible amplification conditions said amplification being universal for the respective vacA and cagA alleles present.

Most particularly, it is an aim of the present invention to select primers enabling the amplification of the highly variable S- and M-regions in the vacA gene, said S-region being comprised between the nucleotide at position 1 and 300, said M-region being comprised between the nucleotides at the position 1450 and 1650, and the highly conserved region between the nucleotide at the position 1 and the nucleotide at the position 250 of the open reading frame of the cagA gene of H.pylori, by preference in a single amplification reaction. It is also an aim of the present invention to provide kits for the detection and/or typing of H.pylori subs.

More particularly, it is an aim of this invention to provide a kit for the detection and/or typing of H.pylori strains directly coupled to the detection and/or the typing of the alleles of the virulence determinant genes present, including at least the vacA gene. Even more particularly, it is an aim of this invention to provide a kit for the detection and/or typing of H.pylori strains based on the detection and/or typing of the alleles of the vacA and cagA genes present.

Most preferentially, it is an aim of this invention to provide a kit for the detection and/or typing Of H.pylori stains based on the detection and/or typing of the highly variable S- and M-regions in the vacA gene and the highly conserved region between the nucleotide at the position 1 and the nucleotide at the position 250 of the cagA gene of H.pylori.

All the aims of the present invention have been met by the following specific embodiments. The selection of the probes (except for probes with SEQ ID NO 35 to 39) according to the present invention is based on the Line Probe Assay (LPA) principle, as exemplified in the Examples section. The LiPA is a reverse hybridization assay using oligonucleotide probes immobilized as parallel lines on a solid support strip (Stuyver et al. 1993; international application WO 94/12670). This approach is particularly advantageous since it is fast and simple to perform. The reverse hybridization format and more particularly the LiPA approach has many practical advantages as compared to other DNA techniques or hybridization formats, especially hen the use of a combination of probes is preferable or unavoidable to obtain the relevant information sought. As such, the LiPA is a particularly appropriate method to detect and or type (micro)-organisms in general and H.pylori in particular. The probes with SEQ ID NO 35 to 39 are designed for use in a DNA Immuno Assay, as shown in example 8. This assay is particularly convenient for a rapid detection method.

It is to be understood, however, that any other type of hybridization assay or hybridization format using any of the selected probes as described further in the invention, is also covered by the present invention.

The reverse hybridization approach implies that the probes are immobilized to a solid support and that the target DNA is labelled in order to enable the detection of the hybrids formed. The following definitions serve to illustrate the terms and expressions used in the present invention.

The target material in the samples envisaged in the present invention may either be DNA or RNA e.g. genomic DNA or messenger RNA or amplified versions thereof. These molecules are also termed polynucleic acids.

The relevant target regions will in principle be all polynucleic acid sequences comprising a virulence determinant gene, said virulence determinant gene being the genetic element involved in enabling, determining, and marking of the infectivity and/or pathogenecity of H.pylori, more specifically all polynucleic acid sequences comprising the virulence determinant genes vacA and cagA, and even more specifically any conserved region in the cagA gene, said conserved region being defined as more being more than 95% identical between alleles of different H.pylori strains, and most specifically the variable S- and M-regions of the vacA gene. In addition to variable sequences, the S-region of the vacA gene also comprises conserved sequences, which may be chosen as target regions for probes for detection—without typing—of H. pylori according to the present invention.

The term “probe” refers to single stranded sequence-specific oligonucleotides which have a sequence which is complementary to the target sequence to be detected.

The term complementary as used herein means that the sequence of the single stranded probe is exactly hybridizing to the sequence of the single-stranded target, with the target being defined as the sequence where the mutation to be detected is located. Since the current application requires the detection of single basepair mismatches, very stringent conditions for hybridization are required, allowing in principle only hybridization of exactly complementary sequences. However, variations are possible in the length of the probes (see below), and it should be noted that, since the central part of the probe is essential for its hybridization characteristics, possible deviations of the probe sequence versus the target sequence may be allowable towards bead and tail of the probe, when longer probe sequences are used. These variations, which may be conceived from the common knowledge in the art, should however always be evaluated experimentally, in order to check if they result in equivalent hybridization characteristics compared to the exactly complementary probes.

Preferably, the probes are about 5 to 50 nucleotides long, more preferably from about 10 to 25 nucleotides. The nucleotides as used in the present invention may be ribonucleotides, deoxyribonucleotides and modified nucleotides such as inosine or nucleotides containing modified groups which do not essentially alter their hybridisation characteristics.

Probe sequences are represented throughout the specification as single stranded DNA oligonucleotides from the 5′ to the 3′ end. It is obvious to the man skilled in the art that any of the below-specified probes can be used as such, or in their complementary form, or in their RNA form (wherein T is replaced by U).

The probes according to the invention can be prepared by cloning of recombinant plasmids containing inserts including the corresponding nucleotide sequences, if need be by cleaving the latter out from the cloned plasmids upon using the adequate nucleases and recovering them, e.g. by fractionation according to molecular weight. The probes according to the present invention can also be synthesized chemically, for instance by the conventional phospho-triester method.

The term “solid support” can refer to any substrate to which an oligonucleotide probe can be coupled, provided that it retains its hybridization characteristics and provided that the background level of hybridization remains low. Usually the solid substrate will be a microtiter plate, a membrane (e.g. nylon or nitrocellulose) or a microsphere (bead) or a chip. Prior to application to the membrane or fixation it may be convenient to modify the nucleic acid probe in order to facilitate fixation or improve the hybridization efficiency. Such modifications may encompass homopolymer tailing, coupling with different reactive groups such as aliphatic groups, NH₂ groups, SH groups, carboxylic groups, or coupling with biotin, haptens or proteins.

The term “labelled” refers to the use of labelled nucleic acids. Labelling may be carried out by the use of labelled nucleotides incorporated during the polymerase step of the amplification such as illustrated by Saiki et al. (1988) or Bej et al. (1990) or labelled primers, or by any other method known to the person skilled in the art. The nature of the label may be isotopic (³²P, ³⁵S, etc.) or non-isotopic (biotin, digoxigenin, etc.).

The team “primer” refers to a single stranded oligonucleotide sequence capable of acting as a point of initiation for synthesis of a primer extension product which is complementary to the nucleic acid strand to be copied. The length and the sequence of the primer must be such that they allow to prime the synthesis of the extension products. Preferably the primer is about 5-50 nucleotides long. Specific length and sequence will depend on the complexity of the required DNA or RNA targets, as well as on the conditions of primer use such as temperature and ionic strenght.

The fact that amplification primers do not have to match exactly with the corresponding template sequence to warrant proper amplification is amply documented in the literature (Kwok et al., 1990).

The amplification method used can be either polymerase chain reaction (PCR; Saiki et al., 1988), ligase chain reaction (LCR; Landgren et al., 1988; Wu & Wallace, 1989; Barany, 1991), nucleic acid sequence-based amplification (NASBA; Guatelli et at, 1990; Compton, 1991), transcription-based amplification system (TAS; Kwoh et al, 1989), strand displacement amplification (SDA; Duck, 1990; Walker et al., 1992) or amplification by means of QB replicase (Lizardi et al., 1988; Lomeli et al., 1989) or any other suitable method to amplify nucleic acid molecules known in the art.

The oligonucleotides used as primers or probes may also comprise nucleotide analogues such as phosphorothiates (Matsukura et al., 1987), alkylphosphorothiates (Miller et al., 1979) Or peptide nucleic acids (Nielsen et al., 1991; Nielsen et al., 1993) or may contain intercalating agents (Asseline et al., 1984).

As for most other variations or modifications introduced into the original DNA sequences of the invention, these variations will necessitate adaptations with respect to the conditions under which the oligonucleotide should be used to obtain the required specificity and sensitivity. However the eventual results of hybridisation will be essentially the same as those obtained with the unmodified oligonucleotides.

The introduction of these modifications may be advantageous in order to positively influence characteristics such as hybridization kinetics, reversibility of the hybrid-formation, biological stability of the oligonucleotide molecules, etc.

The “sample” may be any biological material taken either directly from the infected human being (or animal), or after culturing (enrichment), or collected from any other environment. Biological material may be e.g. expectorations of any kind, broncheolavages, blood, skin tissue, biopsies, lymphocyte blood culture material colonies, liquid cultures, soil, faecal samples, urine, surface water, etc.

The probes of the invention are designed for attaining optimal performance under the same hybridization conditions so that they can be used in sets for simultaneous hybridization; this highly increases the usefulness of these probes and results in a significant gain in time and labour. Evidently, when other hybridization conditions would be preferred, all probes should be adapted accordingly by adding or deleting a number of nucleotides at their extremities. It should be understood that these concommitant adaptations should give rise to essentially the same result, namely that the respective probes still hybridize specifically with the defined target. Such adaptations might also be necessary if the amplified material should be RNA in nature and not DNA as in the case for the NASBA system

For designing probes with desired characteristics, the following useful guidelines known to the person skilled in the art can be applied.

Because the extent and specificity of hybridization reactions such as those described herein are affected by a number of factors, manipulation of one or more of those factors will determine the exact sensitivity and specificity of a particular probe, whether perfectly complementary to its target or not. The importance and effect of various assay conditions, explained further herein, are known to those skilled in the art.

First, the stability of the [probe:target] nucleic acid hybrid should be chosen to be compatible with the assay conditions. This may be accomplished by avoiding long AT-rich sequences, by terminating the hybrids with G:C base pairs. and by designing the probe with an appropriate Tm The beginning and end points of the probe should be chosen so that the length and % GC result in a Tm about 2-10° C. higher than the temperature at which the final assay will be performed. The base composition of the probe is significant because G-C base pairs exhibit greater thermal stability as compared to A-T base pairs due to additional hydrogen bonding. Thus, hybridization involving complementary nucleic acids of higher G-C content will be stable at higher temperatures.

Conditions such as ionic strenght and incubation temperature under which a probe will be used should also be taken into account when designing a probe. It is known that hybridization will increase as the ionic strenght of the reaction mixture increases, and that the thermal stability of the hybrids will increase with increasing ionic strenght. On the other hand, chemical reagents, such as formamide, urea, DMSO and alcohols, which disrupt hydrogen bonds, will increase the stringency of hybridization. Destabilization of the hydrogen bonds by such reagents can greatly reduce the Tₘ. In general, optimal hybridization for synthetic oligonucleotide probes of about 10-50 bases in length occurs approximately 5° C. below the melting temperature for a given duplex Incubation at temperatures below the optimum may allow mismatched base sequences to hybridize and can therefore result in reduced specificity. It is desirable to have probes which hybridize only under conditions of high stringency. Under high stringency conditions only highly complementary nucleic acid hybrids will form; hybrids without a sufficient degree of complementarity will not form Accordingly, the stringency of the assay conditions determines the amount of complementarity needed between two nucleic acid strands forming a hybrid. The degree of stringency is chosen such as to maximize the difference in stability between the hybrid formed with the target and the nontarget nucleic acid. Second, probes should be positioned so as to minimize the stability of the [probe:nontarget] nucleic acid hybrid. This may be accomplished by minimizing the length of perfect complementarity to non-target organisms, by avoiding GC-rich regions of homology to non-target sequences, and by positioning the probe to span as many destabilizing mismatches as possible. Whether a probe sequence is useful to detect only a specific type of organism depends largely on the thermal stability difference between [probe:target] hybrids and [probe:nontarget] hybrids. In designing probes, the differences in these Tm values should be as large as possible (e.g. at least 2° C. and preferably 5° C.).

The length of the target nucleic acid sequence and, accordingly, the length of the probe sequence can also be important. In some cases, there may be several sequences from a particular region, varying in location and length, which will yield probes with the desired hybridization characteristics. In other cases, one sequence may be significantly better than another which differs merely by a single base. While it is possible for nucleic acids that are not perfectly complementary to hybridize, the longest stretch of perfectly complementary base sequence will normally primarily determine hybrid stability. While oligonucleotide probes of different lengths and base composition may be used, preferred oligonucleotide probes of this invention are between about 5 to 50 (more particularely 10-25) bases in length and have a sufficient stretch in the sequence which is perfectly complementary to the target nucleic acid sequence.

Third, regions in the target DNA or RNA which are known to form strong internal structures inhibitory to hybridization are less preferred. Likewise, probes with extensive self-complementarity should be avoided. As explained above, hybridization is the association of two single strands of complementary nucleic acids to form a hydrogen bonded double strand. It is implicit that if one of the two strands is wholly or partially involved in a hybrid that it will be less able to participate in formation of a new hybrid. There can be intramolecular and intermolecular hybrids formed within the molecules of one type of probe if there is sufficient self complementarity. Such structures can be avoided through carefull probe design. By designing a probe so that a substantial portion of the sequence of interest is single stranded, the rate and extent of hybridization may be greatly increased. Computer programs are available to search for this type of interaction. However, in certain instances, it may not be possible to avoid this type of interaction.

The present invention provides in its most general form a method for the detection and/or typing of Helicobacter pylori (H.pylori) strains present in a sample comprising the steps of:

(i) if need be releasing, isolating or concentrating the polynucleic acids in the sample;

(ii) amplifying the polynucleic acids of relevant target regions of the vacA gene and possibly other virulence determinant genes (VDG), with suitable primer pairs, said primers being generally applicable on different H.pylori strains, allowing to amplify said relevant target regions of the VDG preferentially in compatible amplification conditions;

(iii) hybridizing the polynucleic acids obtained in (i) or (ii) with a set of at least two VDG-derived probes, under appropriate hybridization and wash conditions, and with at least one of said probes hybridizing to a conserved region of a VDG of H.pylori, and with at least one of said probes hybridizing to a variable region of vacA;

(iv) detecting the hybrids formed in step (iii);

(v) detecting and/or typing H.pylori strains present in a sample from the differential hybridization signals obtained in step (iv).

Said typing represents the allele-specific detection of a strain according to the VDG alleles present in that particular H.pylori strain. Said virulence determinant genes represent the genetic elements involved in enabling, determining, and marking of the infectivity and/or pathogenicity of said H.pylori strain. Said method is referred to below as “detection/typing method”.

The relevant target regions will be derived from polynucleic acid sequences comprising a virulence determinant gene specific of H.pylori, with said relevant target region being either a conserved region in a VDG, or a variable region of a VGD. The relevant target regions of the virulence determinant genes relate either to any conserved region in known VDG, allowing detection of the presence of this VDG in the H.pylori strains in a sample, or to any variable region in known VDG allowing allele-specific typing of the H.pylori present in a sample.

According to a preferred embodiment of the present invention, step (ii) and (iii) are performed using primers and probes meticulously designed such that they show the desired amplification or hybridization results, when used, if appropriate under compatible amplification or hybridization and wash conditions.

More specifically, the present invention provides a method for the detection and/or typing of H. pylori strains present in a sample with respect to the development of chronic active gastritis and/or gastric and duodenal ulcers and/or gastric adenocarcinomas and/or mucosa-associated lymphoid tissue lymphomas and/or determining eradication therapy.

The cagA gene and the vacA gene are representatives of the virulence determinant genes of H.pylori. Relevant conserved largest regions of alleles of the cagA gene can be used to detect the presence of this gene in H.pylori strains present in a sample. In addition, identified variable regions in alleles of the vacA gene can be used to type in an allele-specific way the respective H.pylori strains. By preference said conserved target regions of alleles of the cagA gene include the region spanning the nucleotide at position 1 to the nucleotide at the position 250 of the open reading frame with said numbering being according to Genbank accessions L11741 (HECMAJANT) or X70039 (HPCAI); also, by preference the identified variable regions of alleles of the vacA gene include the identified S- and M-region of the vacA gene, said S-region being comprised between the nucleotides at position 1 and 300, said M-region being comprised between the nucleotides at the position 1450 and 1650, with said numbering being according to Genbank accessions U05676 or U29401.

Standard hybridization and wash conditions are for instance 2×SSC (Sodium Saline Citrate), 0.1% SDS at 50° C. Other solutions (SSPE (Sodium Saline phosphate EDTA), TMACI (Tetramethyl ammonium Chloride), etc) and temperatures can also be used provided that the specificity and sensitivity of the probes is maintained. If need be, slight modifications of the probes in length or in sequence might have to be carried out in order to maintain the specificity and sensitivity required under the given conditions. Suitable primers can for instance be chosen form a list of primers described below.

In a more preferential embodiment, the above mentioned polynucleic acids from step (ü) are hybridized with at least two, three, four, five or more of the above mentioned cagA- or vacA-derived probes, which cover respectively a conserved region of the cagA gene and a variable region of the vacA gene.

Also, in a more preferential embodiment, the above mentioned polynucleic acids from step (i) and (ii) are hybridized with at least one vacA-derived probe directed to at least one identified variable region of the alleles of the vacA gene, by preference including at least one of the vacA-derived probes SEQ ID NO 2 to 11 and 28 to 34.

It should be stressed that all of the above-mentioned probes, including the allele-specific probes, are contained in the sequence of specific virulence determinant genes of H.pylori, including more particularly the cagA gene or the vacA gene, said probes comprising either a conserved region of the cagA gene, or comprising a variable region of the vacA gene. The probes are preferably designed in such a way that they can all be used simultanously, under the same hybridization and wash conditions. Both criteria imply that preferentially a single amplification and hybridization step is sufficient for the simultanous detection and typing of H.pylori strains present in a sample.

The present invention relates more particularly to a method as defined above wherein step (ii) consists of amplifying the polynucleic acids of relevant target regions in the vacA and cagA gene with suitable sets of primers, said primers being generally applicable on different H. pylori strains, allowing to amplify said relevant target regions in compatible amplification conditions, with said target region being a conserved region in the case of the cagA alleles and a variable region in the case of the vacA alleles, and with said sets of primers being preferentially chosen from the following list of primers as given in Table 1:

| | |
|---|---|
| cagF | (SEQ ID NO 12) |
| cagR | (SEQ ID NO 13) |
| VA1XR | (SEQ ID N0 14) |
| VA1F | (Atherton et al, 1995) |
| M1F | (SEQ ID NO 15) |
| M1R | (SEQ ID NO 16) |
| HPMGF | (SEQ ID NO 17) |
| HPMGR | (SEQ ID NO 18) |
| cagSF | (SEQ ID NO 19) |
| cagSR | (SEQ ID NO 20) |
| cagFN1 | (SEQ ID NO 21) |
| cagRN1 | (SEQ ID NO 22) |
| VAMSFb | (SEQ ID NO 23) |
| VAMSFc | (SEQ ID NO 24) |
| VAMSFd | (SEQ ID NO 25) |
| VAMSFe | (SEQ ID NO 26) |

or sequence variants thereof; with said sequence variants containing deletions and/or insertions and/or substitutions of one or more nucleotides, mainly at their extremities (either 3′ or 5′), and or substitutions of non-essential nucleotides, —being nucleotides not essential in discriminating between alleles—, by others (including modified nucleotides such as inosine), or with said variants conning of the complement of any of the above-mentioned oligonucleotide primers, or with said variants consisting of ribonucleotides instead of deoxyribonucleotides, all provided that the variants can hybridize/amplify specifically with the same specificity as the oligonucleotide primers from which they are derived.

Primers cagF and cagR are derived from two published sequences of cagA alleles (Cocacci et al, 1993; Tummuru et al., 1993). The present invention provides novel nucleic acid sequences encoding 149-154 amino acids of the N-terminus of the cagA protein, as disclosed in FIG. 10 (see also example 5). Based on these novel sequences, improved primers were designed for amplification of a relevant target region of the cagA gene. These primers are:

| | |
|---|---|
| cagSF(forward) | (SEQ ID NO 19) |
| cagSk(reverse) | (SEQ ID NO 20) |

The sequence of these primers is shown in table 1. Study of the alignment of sequences shown in FIG. 10 shows that primers cagSF and cagSR will not hybridize to the polynucleic acids of isolates from East Asia. Therefore, even more improved primers were designed, that will also permit amplification of these sequences. These primers are:

| | |
|---|---|
| cagFN1(forward) | (SEQ ID NO 21) |
| cagRN1(reverse) | (SEQ ID NO 22) |

The sequence of these primers is shown in table 1. Primers cagSF and cagSR can of course be used when amplification of polynucleic acids of isolates from East Asia is not required. Primers M1F. M1R, HPMGF and HPMGR are based on the sequences of the M-region of the vacA gene, shown in FIGS. 2 and 3, said sequences being provided by the present invention. In a second instance, the present invention discloses additional sequences for the M-region, as shown in FIG. 14 (see example 7). Based on these sequences, improved forward primers were designed, that may preferentially be used instead of primer M1F, in combination with reverse primer M1R These primers are:

| | |
|---|---|
| VAMSFb (forward) | (SEQ ID NO 23) |
| VAMSFc (forward) | (SEQ ID NO 24) |
| VAMSFd (forward) | (SEQ ID NO 25) |
| VAMSFe (forward) | (SEQ ID NO 26) |

The sequence of these primers is shown in table 1. In order to obtain amplification of polynucleic acids from a maximal number of isolates, primers VAMSFb, VAMSFc, VAMSFd and VAMSFe should be combined in one PCR reaction.

According to a preferred embodiment, the present invention also relates to a method as defined above wherein step (iii) consists of hybridizing the polynucleic acids obtained in step (ii) with a set of probes, under appropriate hybridization and wash conditions, said set of probes being preferentially applicable in a simultaneous hybridisation assay and comprising at least one probe hybridizing to a conserved region of the cagA gene of H.pylori and at least one probe hybridizing to a variable region of the vacA gene of H.pylori, and more preferentially said set of probes comprising at least one of the following cagA- and vacA-derived probes as defined in Table 2 and in FIGS. 2 to 3:

cag A-derived probe(s):

| | |
|---|---|
| cagApro | (SEQ ID NO 1) |
| cagprobe3 | (SEQ ID NO 27) |

vacA-derived probe(s):

| | |
|---|---|
| P1S1 | (SEQ ID NO 2) |
| P22S1a | (SEQ ID NO 3) |
| P1S1b | (SEQ ID NO 4) |
| P2S1b | (SEQ ID NO 5) |
| P1S2 (VAS2) | (SEQ ID NO 6) |
| P2S2 | (SEQ ID NO 7) |
| P1M1 | (SEQ ID NO 8) |
| P2M1 | (SEQ ID NO 9) |
| P1M2 | (SEQ ID NO 10) |
| P2M2 | (SEQ ID NO 11) |
| P3S1 | (SEQ ID NO 28) |
| P4S1 | (SEQ ID NO 29) |
| P1M1new | (SEQ ID NO 30) |
| P2M1new | (SEQ ID NO 31) |
| P1M2new | (SEQ ID NO 32) |
| P2M2new | (SEQ ID NO 33) |
| P1M3 | (SEQ ID NO 34) |

or sequence variants thereof, with said sequence variants containing deletions and/or insertions and/or substitutions of one or more nucleotides, mainly at their extremities (either 3′ or 5′), and or substitutions of non-essential nucleotides, —being nucleotides not essential in discriminating between alleles—, by others (including modified nucleotides such as inosine), or with said variants consisting of the complement of any of the above-mentioned oligonucleotide probes, or with said variants consisting of ribonucleotides instead of deoxyribonucleotides, all provided that the variants can hybridize specifically with the same specificity as the oligonucleotide probes from which they are derived.

Probe cagApro was derived from published sequences of cagA alleles (Covacci et al., 1993; Tummuru et al. 1993). Based on the above-mentioned novel sequences of the cagA gene (FIG. 10), provided by the present invention, an improved probe was designed:

| | |
|---|---|
| cagprobe3. | (SEQ ID NO 27) |

The sequence of this probe is shown in table 2.

Probes P1S1, P22S1a, P1S1b, P2S1b, P1S2 and P2S2 are based on the sequences of the S-region of the vacA gene (FIG. 2), provided by the present invention These probes are designed to recognize sequences of s1a, s1b and s2 variants, respectively. In a second instance, a larger collection of sequences of the S-region of the vacA gene is disclosed by the present invention, as shown in FIG. 12 (see also example 6). Study of the alignment of these novel sequences, as well as phylogenetic analysis (FIG. 13), reveals the existence of a formerly unknown s1 variant, in addition to the known variants s1a and s1b. This formerly unknown variant is disclosed by the present invention and is denoted s1c. The present invention also provides novel probes, that permit specific hybridization to the sic variant. These probe are:

| | |
|---|---|
| P3s1 | (SEQ ID NO 28) |
| P4s1. | (SEQ ID NO 29) |

The sequence of these probes is shown in table 2.

Probes P1M1, P2M1, P1M2 and P2M2 are based on the sequences of the M-region of the vacA gene that are provided by the present invention and that are shown in FIG. 3. These probes are designed for specific hybridization to the m1 and m2 variants. Alignment of a larger number of sequences of the M-region, also provided by the present invention, reveals the presence of 3 sequences that are different from the m1 and m2 variants (FIG. 14), as shown in example 7. These sequences may represent a novel variant in the M-region. According to the present invention, this variant is denoted m3. Based on the sequences of the M-region that are shown in FIG. 14, novel probes have been designed, these probes being:

| | |
|---|---|
| P1M1new | (SEQ ID NO 30) |
| P2M1new | (SEQ ID NO 31) |
| P1M2new | (SEQ ID NO 32) |
| P2M2new | (SEQ ID NO 33) |

Probes P1M1 new and P2M1 new improve upon probes P1M1 and P2M1 in that they are capable, when used together, to specifically hybridize to all ml sequences shown in FIG. 14. likewise, probes P1M2 new and P2M2 new are improved probes that specifically hybridize to all m2 sequences shown in FIG. 14. In addition, a novel probe that specifically hybridizes to the aforementioned m3 sequences. is provided This probe is:

| | |
|---|---|
| P1M3. | (SEQ ID NO 34) |

The sequences of probes P1M1 new, P2M1 new, P1M2 new, P2M2 new and P1M3 are shown in table 2.

According to another embodiment, the present invention relates to a method for the detection of H.pylori strains present in a sample comprising the steps of:

(i) if need be releasing, isolating or concentrating the polynucleic acids in the sample;

(ii) amplifying the polynucleic acids of a relevant target region of the vacA gene with a suitable primer pair, said primer pair being generally applicable on different H.pylori strains, allowing to amplify said relevant target region of the vacA gene preferentially in compatible amplification conditions;

(iii) hybridizing the polynucleic acids obtained in (i) or (ii) with at least one probe hybridizing to a conserved region of the vacA gene;

(iv) detecting the hybrids formed in step (iii);

(v) determining the presence or absence of H.pylori in a sample from the hybridization signals obtained in step (iv).

Said method is referred to below as the “detection method”.

According to a preferred embodiment, the present invention relates to a method according to the preceding embodiment, wherein step (ii) consists of amplifying the polynucleic acids of a relevant target region in the vacA gene with suitable primers, said primers being generally applicable on different H. pylori strains, allowing to amplify said relevant target region in compatible amplification conditions, with said target region being a conserved region, with said primers preferentially being VA1F and VAL1X (SEQ ID NO14), or sequence variants thereof, with said sequence variants containing deletions and/or insertions and/or substitutions of one or more nucleotides, mainly at their extremities (either 3′ or 5′), and or substitutions of non-essential nucleotides, —being nucleotides not essential in discriminating between alleles, by others (including modified nucleotides such as inosine), or with said variants consisting of ribonucleotides instead of deoxyribonucleotides, all provided that the variants can hybridize/amplify specifically with the same specificity as the oligonucleotide primers from which they are derived.

According to an even more preferred embodiment, the present invention relates to a method according to any of the two preceding embodiments, wherein step (iii) consists of hybridizing the polynucleic acids obtained in step (ii) with a set of probes, under appropriate hybridization and wash conditions, said set of probes being preferentially applicable in a simultaneous hybridisation assay and comprising at least one probe hybridizing to a conserved region of the vacA gene of H.pylori, and more preferentially said set of probes comprising wing at least one of the following vacA-derived probes:

| | |
|---|---|
| HpdiaS1 | (SEQ ID NO 35) |
| HpdiaS2 | (SEQ ID NO 36) |
| HpdiaS3 | (SEQ ID NO 37) |
| HpdiaS4 | (SEQ ID NO 38) |
| HpdiaS5 | (SEQ ID NO 39) |

or sequence variants thereof with said sequence variants containing deletions and/or insertions and/or substitutions of one or more nucleotides, mainly at their extremities (either 3′ or 5′), and or substitutions of non-essential nucleotides, —being nucleotides not essential in discriminating between alleles—, by others (including modified nucleotides such as inosine), or with said variants consisting of the complement of an, of the above-mentioned oligonucleotide probes, or with said variants consisting of ribonucleotides instead of deoxyribonucleotides, all provided that the variants can hybridize specifically with the same specificity as the oligonucleotide probes from which they are derived.

According to another embodiment, the present invention relates to a probe composition for use in any detection/typing method as defined above, said composition comprising at least one probe hybridizing to a conserved region of a VDG of H.pylori, and at least one probe hybridizing to a variable region of vacA, and more preferentially said probes being derived from the polynucleic acid sequences of the vacA and/or cagA gene of H.pylori, and most preferentially said probes been chosen from SEQ ID NO 1 to 11 and 27 to 34, or sequence variants thereof with said sequence variants containing deletions and/or insertions and/or substitutions of one or more nucleotides, mainly at their extremities (either 3′ or 5′), and or substitutions of non-essential nucleotides, —being nucleotides not essential in discriminating between alleles—, by others (including modified nucleotides such as inosine), or with said variants consisting of the complement of any of the above-mentioned oligonucleotide probes, or with said variants consisting of ribonucleotides instead of deoxyribonucleotides, all provided that the variants can hybridize specifically with the same specificity as the oligonucleotide probes from which they are derived.

According to another embodiment, the present invention relates to a probe composition for use in any detection method as defined above, said composition comprising at least one probe hybridizing to a conserved region of the vacA gene of H.pylori, and most preferentially said probe being chosen from SEQ ID NO 35 to 39, or sequence variants thereof with said sequence variants containing deletions and/or insertions and/or substitutions of one or more nucleotides, mainly at their extremities (either 3′ or 5′), and or substitutions of non-essential nucleotides, —being nucleotides not essential in discriminating between alleles—, by others (including modified nucleotides such as inosine), or with said variants consisting of the complement of any of the above-mentioned oligonucleotide probes, or with said variants coning of ribonucleotides instead of deoxyribonucleotides; all provided that the variants can hybridize specifically with the same specificity as the oligonucleotide probes from which they are derived.

According to another embodiment, the present invention relates to a composition comprising at least one suitable oligonucleotide amplification primer, allowing to amplify the polynucleic acids of the relevant target regions of the respective VDG, said suitable primers being generally applicable with different H.pyiori strains and allowing the amplification of said relevant target regions to be used in compatible amplification conditions, and more preferentially said primers allowing the amplification of a conserved region of the cagA gene and a region of the vacA gene comprising conserved and/or variable target regions, and most preferentially said primers being selected from SEQ ID NO 12 to 26, or sequence variants thereof with said sequence variants containing deletions and/or insertions and/or substitutions of one or more nucleotides, mainly at their extremities (either 3′ or 5′), and or substitutions of non-essential nucleotides, —being nucleotides not essential in discriminating between alleles—, by others (including modified nucleotides such as inosine), or with said variants consisting of the complement of any of the above-mentioned oligonucleotide primers, or with said variants consisting of ribonucleotides instead of deoxyribonucleotides, all provided that the variants can hybridize specifically with the same specificity as the oligonucleotide primers from which they are derived.

According to an even more specific embodiment the present invention relates to a probe being derived from the polynucleic acid sequences of the vacA and/or cagA gene of H.pylori, and with said probe being chosen from SEQ ID NO 1 to 11 and 27 to 39, or sequence variants thereon with said sequence variants containing deletions and/or insertions and/or substitutions of one or more nucleotides, mainly at their extremities (either 3′ or 5′), and or substitutions of non-essential nucleotides, —being nucleotides not essential in discriminating between alleles—, by others (including modified nucleotides such as inosine), or with said variants consisting of the complement of any of the above-mentioned oligonucleotide probes, or with said variants consisting of ribonucleotides instead of deoxyribonucleotides, all provided that the variants can hybridize specifically with the same specificity as the oligonucleotide probes from which they are derived.

According to yet another even more preferred embodiment, the present invention relates to an oligonucleotide amplification primer allowing the amplification of a region of the cagA gene or a region of the vacA gene of H.pylori, and with said primer being selected from SEQ ID NO 12 to 26, or sequence variants thereof with said sequence variants containing deletions and/or insertions and/or substitutions of one or more nucleotides, mainly at their extremities (either 3′ or 5′), and or substitutions of non-essential nucleotides, —being nucleotides not essential in discriminating between alleles—, by others (including modified nucleotides such as inosine), or with said variants consisting of the complement of any of the above-mentioned oligonucleotide primers, or with said variants consisting of ribonucleotides instead of deoxyribonucleotides, all provided that the variants can hybridize/amplify specifically with the same specificity as the oligonucleotide primers from which they are derived.

According to another embodiment the present invention relates to a method as defined above for the detection and/or typing of alleles of VDG of H.pylori, more preferentially alleles of the cagA and vacA gene of H.pylori, present in a sample using a set of probes and/or primers specially designed to detect and/or to amplify and/or to type the said alleles, with said probes and primers being defined above.

According to another embodiment, the present invention relates to a method as defined above for the detection of alleles of VDG of H. pylori, more preferentially alleles of the vacA gene of H.pylori, present in a sample using a set of probes and/or primers specially designed to detect and/or to amply the said alleles, with said probes and primers being defined above. In order to detect and/or type the H.pylori strains present in the sample, using the above set of oligonucleotide probes, any hybridization method known in the art can be used (conventional dot-blot, Southern blot. sandwich, chip-based, etc). In order to obtain fast and easy results if a large number of probes is involved, a reverse hybridization format may be most convenient. According to a preferred embodiment, a selected set of probes are immobilized onto a solid support.

In another preferred embodiment, a selected set of probes are immobilized to a membrane strip. Said probes may be immobilized individually or as mixtures on the solid support. A specific and very user-friendly embodiment of the above-mentioned preferential method is the LiPA-method, where the above-mentioned set of probes is immobilized in parallel lines on a membrane. as further described in the examples.

Alternatively, detection—without typing—of H. pylori strains may be performed conveniently by use of the DNA Enzyme Immuno Assay (DEIA). The principle of this assay as well as an application based on the detection of a conserved part of the S-region of the vacA gene is outlined in example 8.

Some of the above described probes are directed towards nucleic acid sequences already discribed in the prior art. However, as illustrated in the examples, nucleic acid sequences of VDG of a large number of new isolates of H.pylori were disclosed for the first time in this invention, providing valuable new information necessary to succesfully design suitable probes with respect to detecting and more importantly to typing H.pylori strains. These new H. pylori sequences also form part of the present invention.

Moreover, previously designed primers and probes by other autors (Atherton et al, 1995) are shown in the examples to be less appropriate in typing H.pylori strains in a sample.

This invention also provides for probes and primers(sets) which are designed to specifically detect or amplify the respective VDG alleles of the new isolates, and provides moreover methods and kits for applying said primers or probes in the detection and/or typing of H.pylori strains in a sample.

The present invention also provides for a set of primers, allowing amplification of the conserved region spanning the region between the nucleotide at position 1 to the nucleotide at position 250 of the cag gene of H.pylori. The set of primers comprises for instance:

cagf and cagr (SEQ ID N° 11 and 12)

Also, the present invention provides sets of primers covering the variable S- and/or M-regions of the vacA gene of H.pylori, said S-region being comprised between the nucleotide at position 1 and 300 and comprising conserved sequences in addition to variable sequences, said M-region being comprised between the nucleotides at the position 1450 and 1650, with said primers for instance being:

VA1-F and VA1-XR (Atherton et al., 1995 and SEQ ID N° 15) M1F and M1R (SEQ ID N° 16 and 17)

The invention also provides methods and kits to apply the above described primers sets directed to particular regions of VDG genes, e.g the cagA and vacA genes, simultaneously under identical amplification, hybridisation and washing conditions.

The primers according to the present invention may be labeled with a label of choice (e.g. biotine). Different target amplification systems may be used, and preferentially PCR-amplification, as set out in the examples. Single-round or nested PCR may be used.

According to yet another embodiment, the present invention relates to a solid support, preferentially a membrane strip, carrying on its surface, at least one probe as defined above. According to another embodiment, the present invention relates to a kit for detecting and/or typing H. pylori strains in a sample liable to contain it, comprising the following components:

when appropriate at least one oligonucleotide primer as defined;

at least one probe as defined above, with said probe and/or other probes applied being by preference immobilized on a solid support;

a buffer or components necessary to produce the buffer enabling an amplification or a hybridization reaction between these probes and the amplified products;

when appropriate a means for detecting the hybrids resulting from the preceding hybridization

The term “hybridization buffer” means a buffer enabling a hybridization reaction to occur between the probes and the polynucleic acids present in the sample, or the amplified products, under the appropriate stringency conditions.

The term “washing solution” means a solution enabling washing of the hybrids formed under the appropriate stringency conditions.

The present invention also relates to isolated vacA polynucleic acid sequences defined by SEQ ID NO 40 to 91 and SEQ ID NO 115 to 276 or any fragment thereof that can be used as a primer or as a probe in a method for detection and/or typing of one or more vacA alleles of H. pylori.

The present invention also relates to isolated cagA polynucleic acid sequences defined by SEQ ID NO 92 to 114 or any fragment thereof, that can be used as a primer or as a probe in a method for detection and/or typing of one or more cagA alleles of H. pylori.

The present invention also relates to a vacA protein fragment encoded by any of the nucleic acids with SEQ ID NO 40 to 91 and SEQ ID NO 115 to 276 or any subfragment of said vacA protein fragment, with said subfragment consisting of at least 5, 6, 7, 8, 9, 10, 11, 12, 13, 14, 15, 16, 17, 18, 19, 20, 21, 22, 23, 24 or 25 contiguous amino acids of a vacA protein.

The present invention also relates to a cagA protein fragment encoded by any of the nucleic acids with SEQ ID NO 92 to 114, or any subfragment of said cagA protein fragment, with said subfragment contisting of at least 5, 6, 7, 8, 9, 10, 11, 12, 13, 14, 15, 16, 17, 18, 19, 20, 21, 22, 23, 24 or 25 contiguous amino acids of a cagA protein.

The following examples serve to illustrate the present invention and are in no way to be construed as liming the scope of this invention. It should also be noted that the contents of all references referred to in this invention are hereby incorporated by reference.

### LEGENDS TO TIRE FIGURES

FIG. 1: Schematic overview of the S- and M-region of the vacA gene of H.pylori and indication of the overall position of the relevant primers.

FIG. 2a: DNA sequence alignment of the S-region S1a/b of various H.pylori strains.

FIG. 2b: DNA sequence alignment of the S-region S2 of various H.pylori strains.

FIG. 3a: DNA sequence alignment of the M-region M1 of various H.pylori strains.

FIG. 3b: DNA sequence alignment of the M-region M2 of various H.pylori strains.

FIG. 4: Agarose gel-electrophoresis of the amplification products using as starting material DNA from the gastric biopsy 18 and primers indicated in example 1.

FIG. 5: Agarose gel-electrophoresis of the amplification products using as starting material DNA from the gastric biopsy 41 and primers indicated in example 1.

FIG. 6: Agarose gel-electrophoresis of the amplification products using as starting material DNA from the gastric biopsy F67 and primers indicated in example 1.

FIG. 7: Agarose gel-electrophoresis of the amplification products using as starting material DNA from the gastric biopsy 25 and primers indicated in example 1.

FIG. 8: LIPA outline where the probes indicated in the figure are according to table II and primers according to example 3.

FIG. 9: Multiplex PCR with vacA as well as cagA primers. For vacA primer set G was used (FIG. 1); for cagA primers cagf and cagr were used. The isolate shown in the first two lanes contains s1 and m1 alleles and is caga+. The isolate shown in lanes 4 and 5 (counting from left) contains a multiple infection.

FIG. 10: Alignment of cagA nucleic acid sequences, encoding the N-terminus of the cagA protein. The position of some cagA primers is indicated. Hyphens indicate gaps introduced to obtain optimal alignment. Asterisks below the alignment indicate identical nucleotides. Dots below the alignment indicate partial conservation.

FIG. 11: Phylogenetic tree of cagA amino acid sequences. The 16 sequences counting from the top represent the first variant, occurring mainly in Europe and in Australia. USA123 and USA39 are stains from the USA, having an intermediate position. The 7 sequences counting from the bottom (HK7 to HKTh8828) represent a variant that is mainly found in Far East Asia.

FIG. 12: Alignment of nucleic acid sequences of part of the S-region of the vacA gene. The sequences are grouped according to the variant that they belong to. A larger number of sequences is shown the in FIGS. 2a and 2b. The variants are from top to bottom; s2, s1c, s1b and s1a. Hyphens indicate that at that position the nucleotide is identical to that in the sequence of stain 29401. Dots indicate a gap in the sequence that was introduced to preserve alignment.

FIG. 13: Phylogenetic analysis of nucleic acid sequences of part of the S-region of the vacA protein. The variants are indicated.

FIG. 14: Alignment of nucleic acid sequences of part of the M-region of the vacA gene. A larger number of sequences is shown than in FIGS. 3a and 3b. Hyphens and dots as in FIG. 12.

FIG. 15: Phylogenetic a of nucleic acid sequences of part of the M-region of the vacA protein The variants are indicated.

### EXAMPLES

### Example 1

### Evaluation of the Use of the Primers Described by Atherton et al., 1995 in typing H.pylori Strains within the Framework of Large Scale Clinical Trials

1.1 Comparison of VacA Genotyping Methods.

The efficiency of the vacA genotyping as described by Atherton et al. (1995) was compared to the efficacy as described in the present invention. The method as described by Atherton comprises 6 different PCR reactions:

A. Using primers VA1F and VA1R, to distinguish s1 and s2 alleles.

B. Using primers SS1F and VA1R, to amplify s1a sequences.

C. Using primers SS3F and VA1R, to amplify s1b sequences.

D. Using primers SS2F and VA1R, to amplify s2 sequences.

E. Using primers VA3F and VA3R, to amplify m1 sequences.

F. Using primers VA4F and VA4R, to amplify m2 sequences.

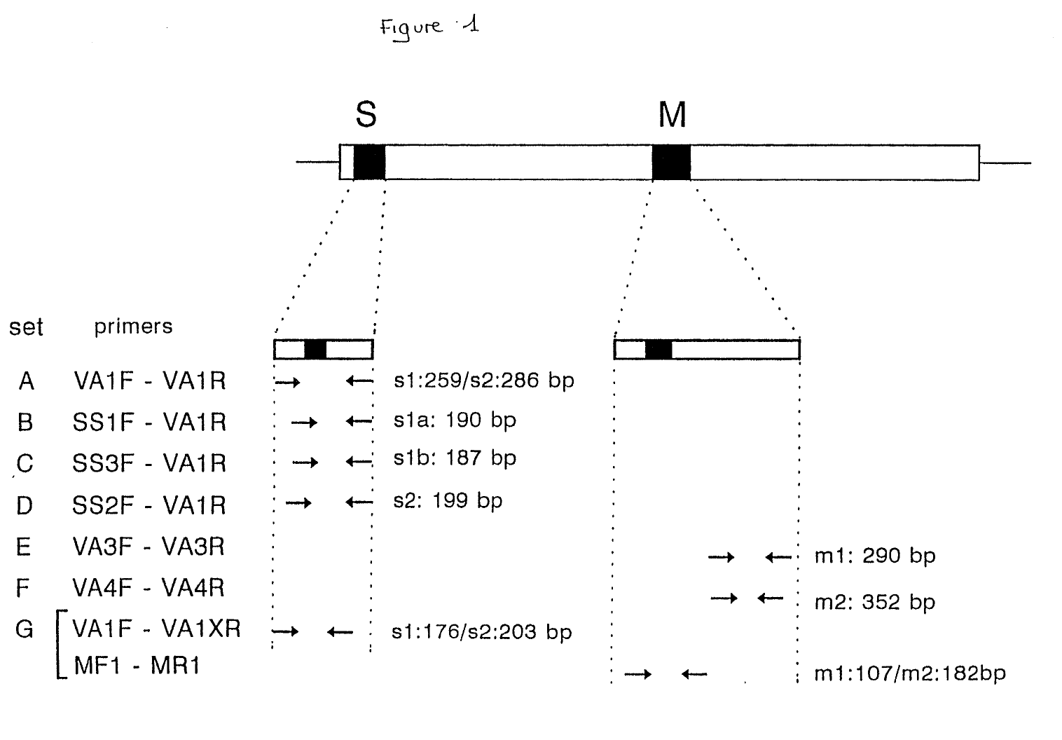

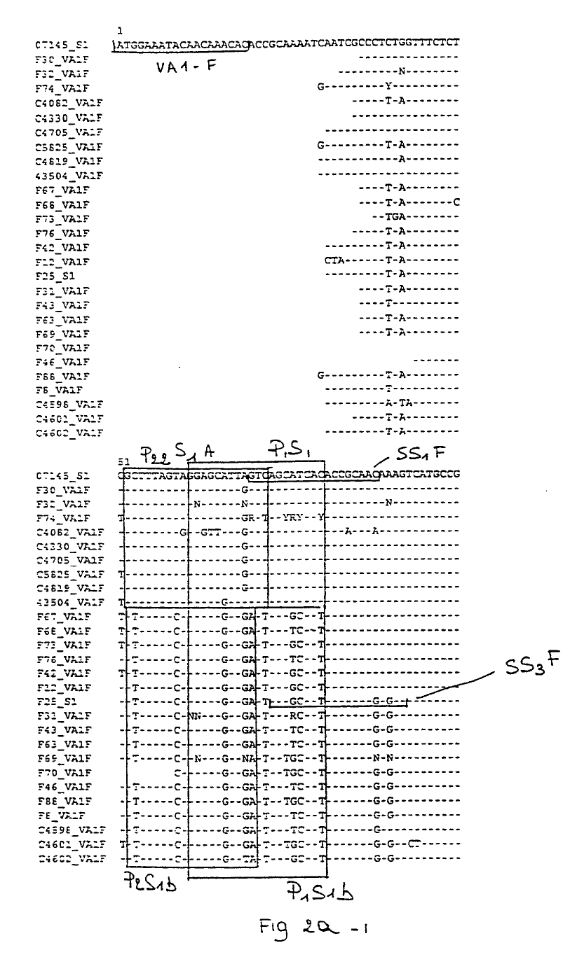

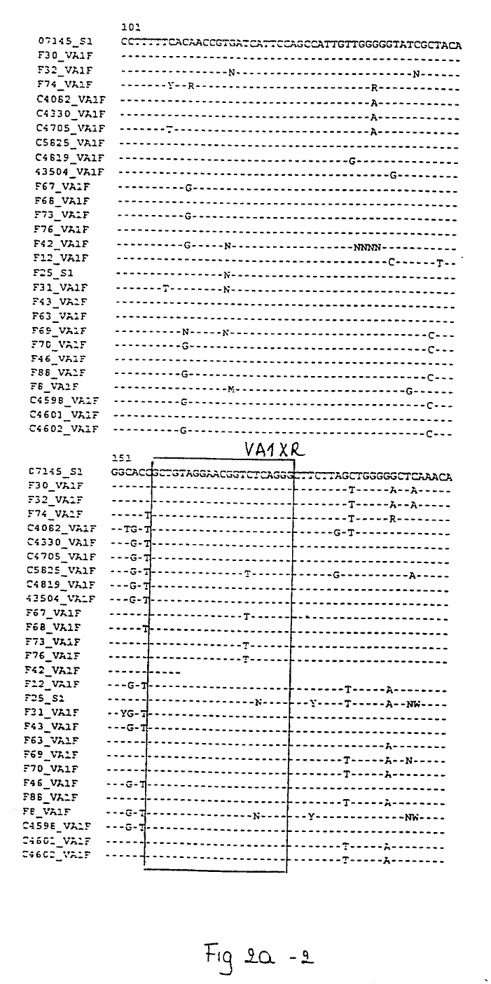

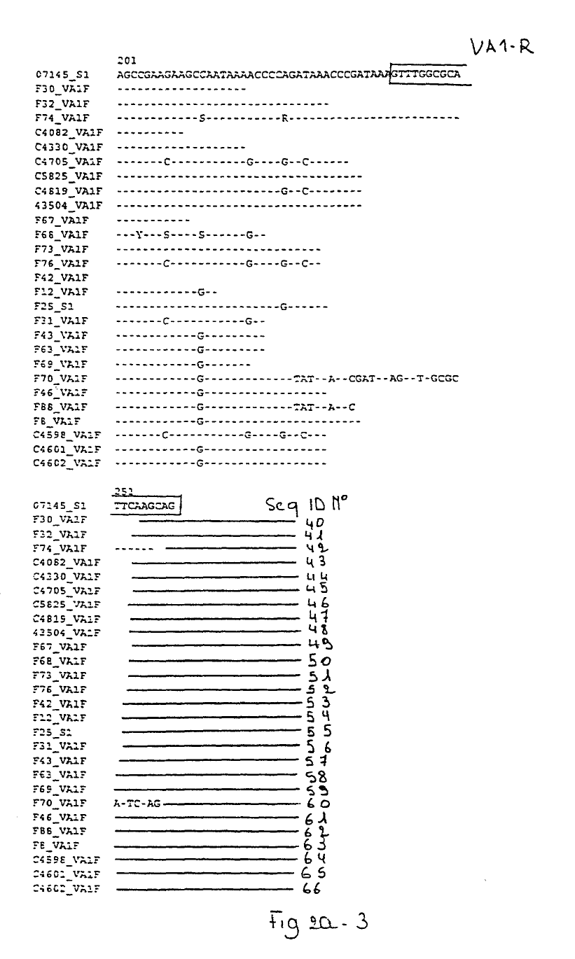

FIG. 1 shows a schematic representation of all primers involved in vacA 1 ping. Identification of the PCR products is based on visual inspection of DNA bands on an agarose gel.

1.2. Problems with the Atherton System:

The s-region:

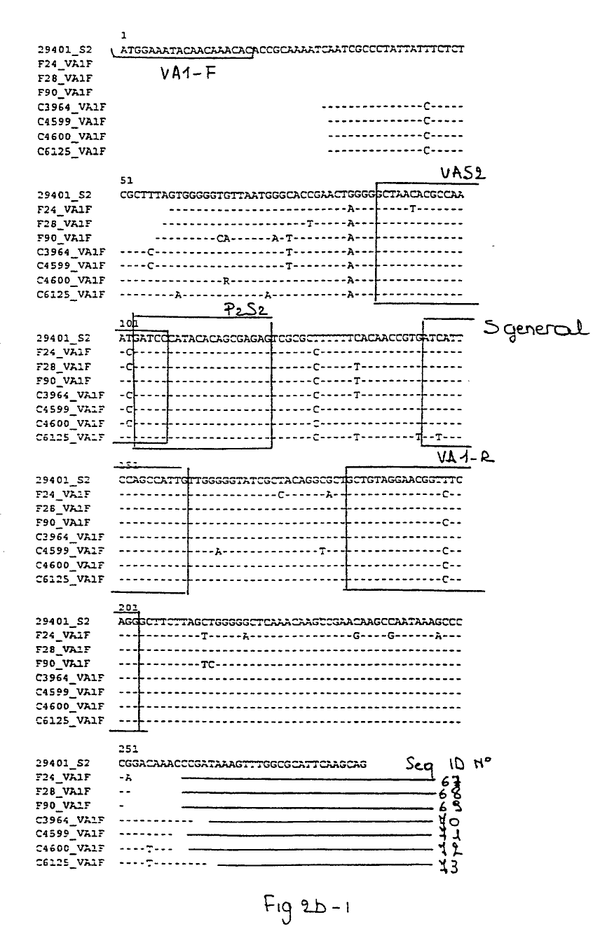

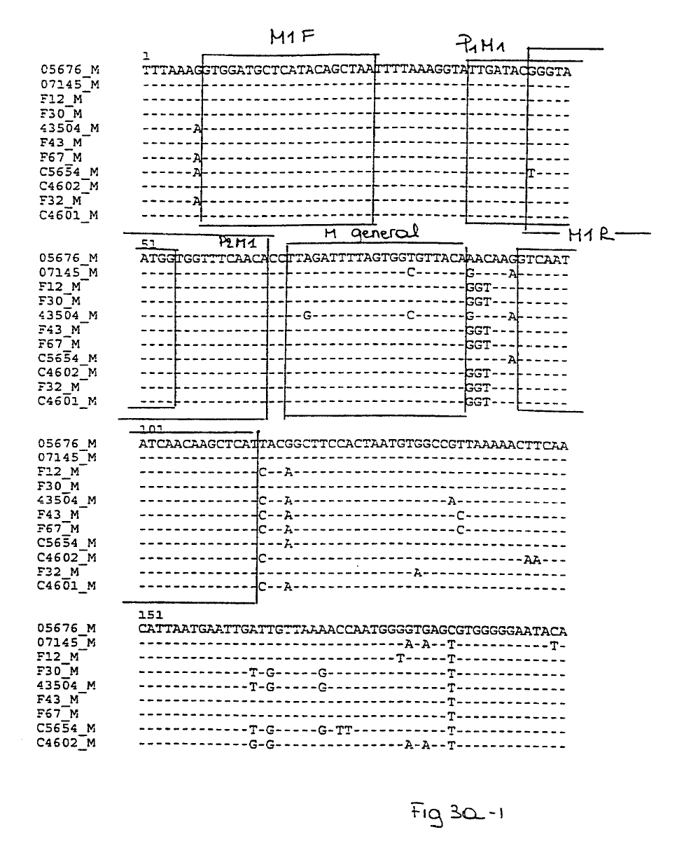

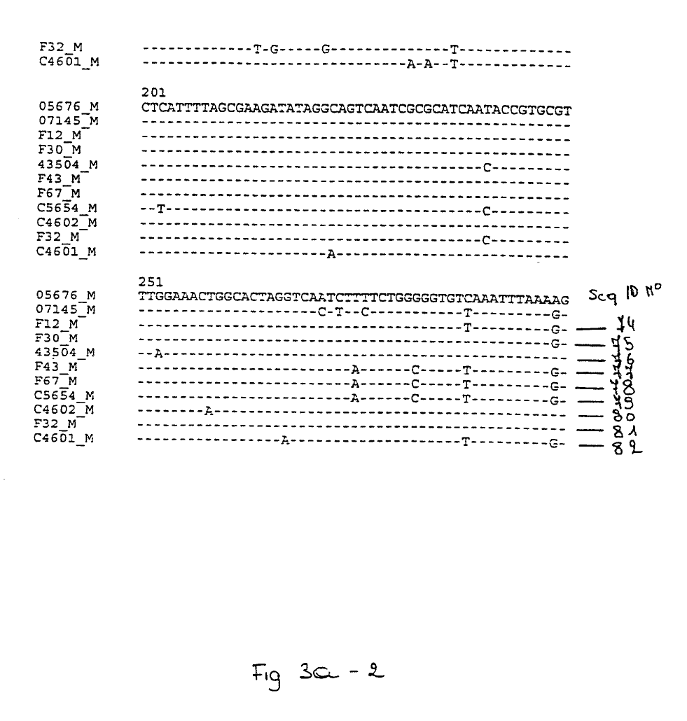

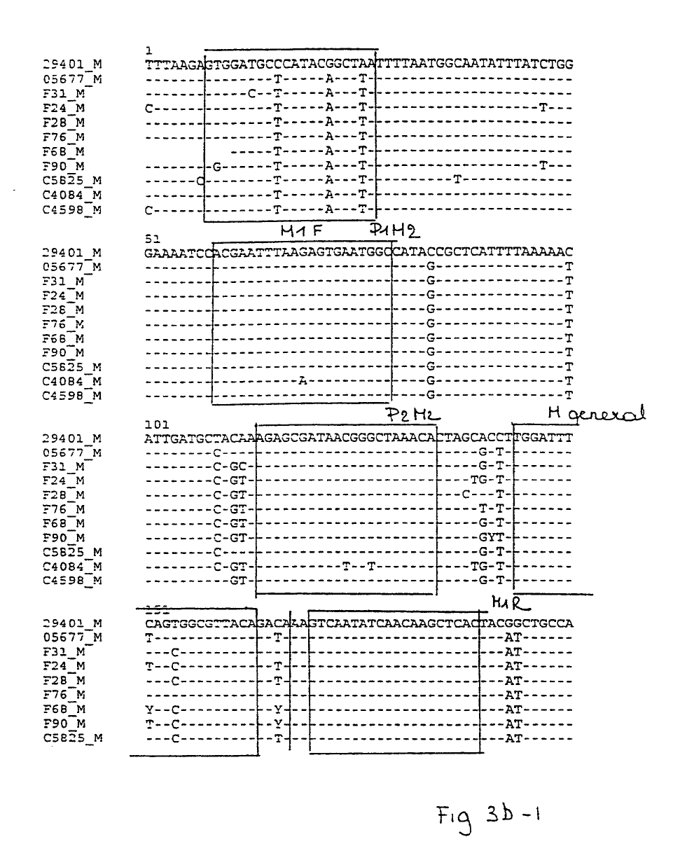

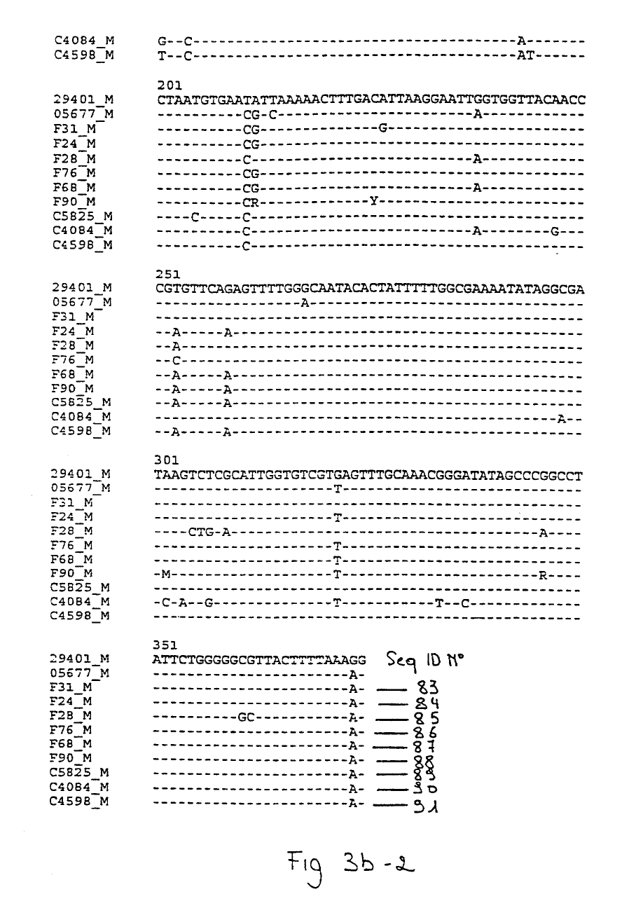

Based on the sequence alignments from European isolates. as shown in FIGS. 2a and 2b it is clear that primers SS1F, SS2F, and SS3F may contain several mismatches to their respective target sequences. This may hamper proper annealing of the primers and may lead to amplification of spurious bands. The target sequence for primer SS3F (aimed at detection of the s1b allele). contains two crucial mismatches at the 3′ end of the primer in some isolates (e.g. in isolates F67, F68, F73, F76, F42, F12).

F67 (see below) showed amplification with primer SS1F and VA1R, whereas amplification with SS3F and VA1R was negative, suggesting the presence of the s1a genotype. However, PCR/LiPA analysis showed the presence of genotype s1a, which was confirmed by sequence analysis.

Primer SS2F, aimed at s2 sequences, results in amplification of a specific bands (see e.g. figure photo 1 & 2, in case of primerset D).

The m-region:

As described by Atherton el al., (1995) typing of the m-region was initially based on hybridization with two specific DNA probes, ie. pCTB4 and VA6 for the M1 and M2 variant, respectively.

From the published nucleotide alignments of the vacA sequences from strain 60190 (type M1) and Tx30a (U29401; type M2). it is obvious that these two probes cover a region of substantial variation.

Moreover, the M1 variant shows a deletion (around position 2340 of the 60190 sequence), compared to the M2 variant. One might envisage that this region of deletion/insertion (or to the S-region) is of major importance to discriminate M1 and M2. However, the PCR primers for specific detection of M1 and M2 are aimed at a different region of the vacA gene, which is more downstream (between positions 2750 and 3030 of the 60190 sequence)and is not covered by the original DNA probes.

We have analysed the individual PCR primers by sequence alignments to the M1 and M2 sequences. We noticed that the 3′ ends of several primers described by Atherton et al., are not completely unique in the vacA gene.

Primer VA3-R shows homology to sequences:

in strain 60190 (Genbank Seq U05676; m1-type):

around pos 229 (6 nt at the 3′ end)

around pos 839 (6 nt at the 3′ end)

around pos 3011 (target sequence, 100%)

around pos 4653 (6 nt at the 3′ end)

in strain Tx30a:

around pos 4271 (6 nt at the 3′end)

Primer VA4-F shows homology to sequences:

in strain Tx30a (GenBank Seq # U29401; m2-type):

around pos 231 (7 nt at the 3′ end)

around pos 1907 (8 nt at the 3′ end)

around pos 2297 (target sequence, 100%)

around pos 2594 (9 nt at the 3′ end)

Especially the homologies at the very 3′ end may hamper the specificity of these primers. Some spurious bands were obtained when using these primers. Moreover, these primers failed to yield any amplification product in several isolates or biopsies (e.g., biopsy 41, see below). This has been observed before (Maeda, S, K Ogura. M. Ishitom F. Kanai H. Yoshida, S. Ota, Y. Shiratori, and M. Omata. abstract # 492: Diversity of Helicobacter pylori E acA gene in Japanese strains —high cytotoxin activity type s1 is dominant in Japan, Digestive Disease Weeks San Fransisco, May 1996).

We have analysed the M1 and M2 region of be vacA allele from multiple H.pylori strains by DNA sequencing upon PCR amplification using as primers HPMGF and HPMGR (see FIGS. 3a and 3b). Based on these sequences new primers had to be developed for vacA genotyping in a multiplex PCR, as described in example 4.

1.3. Comparative Results are Shown in Figures and Discussed Below:

The respective primers used by Atherton et al. (1995) were used in A-F while primerset G. comprised the newly designed set of primers comprising VA1F, VA1XR, M1F, and M1R disclosed for the first time in this invention. AL of these primers are new as such, except VA1F which was disclosed by Atherton et al., 1995.

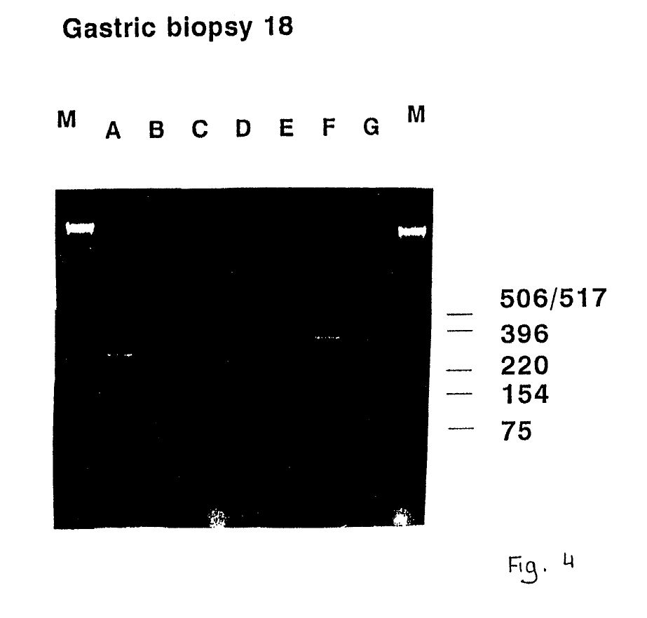

Biopsy #18 (see FIG. 4)

| | | |
|---|---|---|
| A | s1/s2 | S1 |
| B | s1a | + |
| C | s1b | − |
| D | s2 | − (note the background) |
| E | m1 | − |
| F | m2 | + |
| G | multi | s1/m2 |

From this biopsy, the expected fragments were amplified, consistent with a s1a/m2 genotype. Multiplex PCR followed by LiPA as described in the present invention, yielded an identical result.

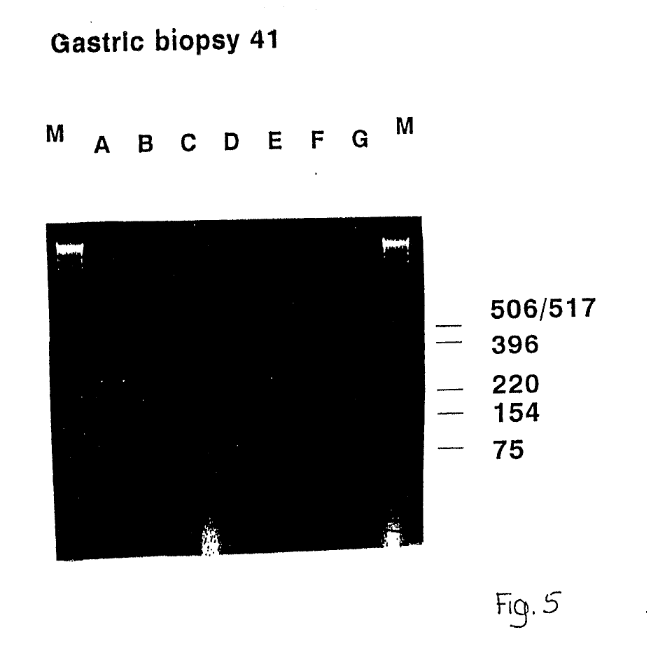

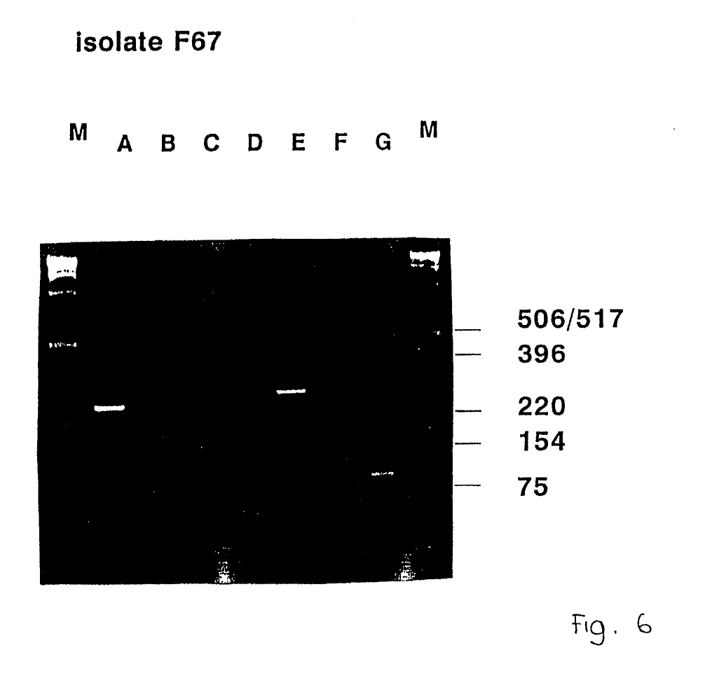

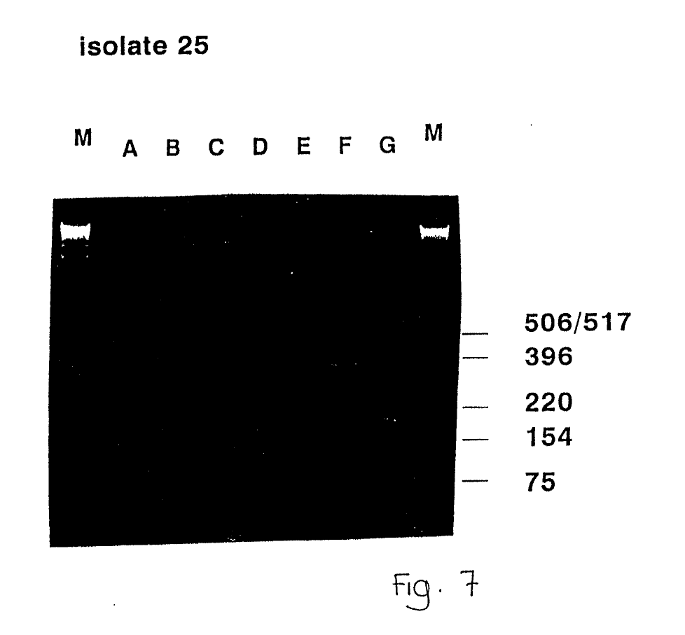

Biopsy #41 (see FIG. 5)

| | | |
|---|---|---|
| A | s1/s2 | s1 |
| B | s1a | + |
| C | s1b | − |
| D | s2 | − (note the background) |
| E | m1 | − |
| F | m2 | − |
| G | multi | s1/m1 |

From this biopsy, only the s-region could be typed by the method of Atherton et al. Amplification with the m1 and m2-specific primers did not yield any visible DNA product. However, by the multiplex PCR followed by LIPA as described in the present invention. a s1a, m1 genotype was detected.

Isolate F67 (see FIG. 6)

| | | |
|---|---|---|
| A | s1/s2 | s1 |
| B | s1a | − |
| C | s1b | − |
| D | s2 | ÷ (note the background) |
| E | m1 | − |
| F | m2 | − |
| G | multi | s1/m1 |

LiPA showed the presence of a s1b, instead of s1a. This was confirmed by sequence analysis,

1.4, Detection of Multiple Infections

Biopsy 25 (see FIG. 7)

| | | |
|---|---|---|
| A | s1/s2 | both |
| B | s1a | − |
| C | s1b | + |
| D | s2 | + (note the background) |
| E | m1 | + |
| F | m2 | + |
| G | multi | s1/s2/m1/m2 |

LiPA analysis revealed the presence of s1b/s2/m1/m2 mixed genotypes.

### Example 2

### Identification and Amplification of a Conserved Region of the cagA Gene Fragment in H.pylori; Designing Primers and a cagA-Derived Probe Allowing to Detect H.pylori in a Sample Through Reverse Hybridization

The establishment of the experimental conditions in order to set up a reverse hybridisation assay in case of the cagA gene comprised i) a theoretical evaluation of suitable probes and primers based upon nucleic acid sequence comparisons using standard DNA analysing computer programmes, and ii) an experimental evaluation and adjustment of the primers and probes to the conditions set for the reverse hybridization technology.

Comparison of two published nucleic acid sequences of cagA alleles of different H.pylori strains demonstrated that the region between nucleic acids 17 to 113 is highly conserved (Covacci et al., 1993; Tummuru et al, 1993) and said region could be used for positive identification of the presence of the cagA gene in a certain H.pylori strain.

A set of primers was designed as follows:

cagF (bp 17 to 40)
cagR (bp 178-199)

Both primers are new primer sequences, described by the current invention (see table I). These primers can be labeled with a label of choice (e.g. biotine). Different primer-based target-amplification systems may be used. For amplification using the PCR, the conditions used in case of a single-round amplification with above primers cagF and cagR involve 40 cycles of 1 min/95° C., 1 min/55° C. 1 min/72° C. followed by a final extension for 5 min at 72° C.

The PCR reaction mixture was as follows:

1 μI DNA sample containing H.pylori or control DNA

10 μl 10×polymerase mix (final concentration 10 m Tris HCl pH 19.0, 50 mM KCl 2.5 mM MgCl, 0.01% gelatin, and 0.1% Triton)

20 μl deoxyribonucleotide mix (final concentration 200 μM each)

1 μl Super Taq polymerase (0.25 U/μl)

1 μl forward primer (50 pmole(μl)

1 μl reverse primer (50 pmoles/μl)

66 μl water

100 μl

Amplification products were analysed on an agarose gel, stained with ethidiumbromide and visualized under UV. The amplified product obtained using 1 μl of H.pylori DNA as starting material and above primers consisted of a single band with approximatively molecular weight 0.18 Kb, in agreement with the expected size of 183 bp. Control samples containing DNA from cag(−) H.pylori strains or other bacterial species did not yield any amplification product (data not shown).

The uniformity of the amplified product was verified through DNA sequencing applying standard sequencing techniques. A 100% match with the described region could be demonstrated (data not shown).

Also, a number of probes were tested in order to determine optimal hybridization between the above amplified product and the said probes under standardized hybridization and washing conditions applied in the reverse hybridisation assay.

The below probes tested were chosen from the list indicated in table II. Said probes were immobilized onto a solid support as described in example 3. The amplified product obtained with said above primers was hybridized to the respective probes applying the same conditions as outlined in example 3. Most optimal results were obtained with probe cagApro (SEQ ID N° 12), which can thus be used as a positive identification of the presence of the cagA gene in H. pylori strains, in combination with the above primers under the conditions of the below reverse hybridization assay.

### Example 3

### Identification and Amplification of Variable Target Regions of the vacA Gene in H.pylori; Designing Primers and a vacA-Derived Probe Allowing to Detect and/or Type H.pylori in a Sample Through Reverse Hybridization

The establishment of the experimental conditions in order to set up a reverse hybridisation assay in case of the vacA gene comprised i) a theoretical evaluation of suitable probes and primers based upon nucleic acid sequence comparisons using standard DNA analysing computer programmes, and ii) an experimental amplification of the various variable regions, DNA sequence analysis of the respective amplified fragments, designing allele-specific probes and appropriate primers, and the evaluation and the adjustment of the primers and probes to the conditions applicable in the reverse hybridization technology.

Recently Atherton et al. (1995) demonstrated the presence of two variable regions in the vacA gene, being the S- and M-region. Primers were designed in order to amplificate specifically alleles of the vacA with variable S- and M-regions.

In this invention, a large number of additional nucleic acid sequences spanning both the said S- or M-region were obtained upon DNA sequence analysis of PCR amplification of said regions. These data are new and are being disclosed here for the first time in the present invention (see FIGS. 2 and 3).

In order to obtain amplification products spanning either the S- or the M-region of the vacA gene, the following set of primers was used:

| | |
|---|---|
| S-region: | VA1-F (see Atherton et al., 1995) |
| | VA1-R (see Atherton et al., 1995) |
| M-region: | HPMGF (CACAGCCACTTTCAATAACGA) |
| | HPMGR (CGTCAAAATAATTCCAAGGG) |

These primers can be labeled with a label of choice (in this case biotine was used). Different primer-based target-amplification systems may be used. For amplification using the PCR the conditions used in case of a single-round amplification with above primers, involve 40 cycles of 1 min/95° C., 1 min/55° C. 1 min/72° C. followed by a final extension for 5 min at 72° C.

The PCR reaction mixture was as follows:

1 μl DNA sample, containing H.pylori or control DNA

10 μl 10×polymerase mix (final concentration 10 mM Tris HCl, pH 9.0, 50 mM KCl 2.5 mM MgCl₂, 0.01% gelatin, and 0.1% Triton)

20 μl deoxyribonucleotide mix (final concentration 200 μM each)

1 μl Super Taq polymerase (0.25 U/μl)

1 μl forward primer (50 pmoles/μl)

1 μl reverse primer (50 pmoles/μl)

66 μl water

100 μl

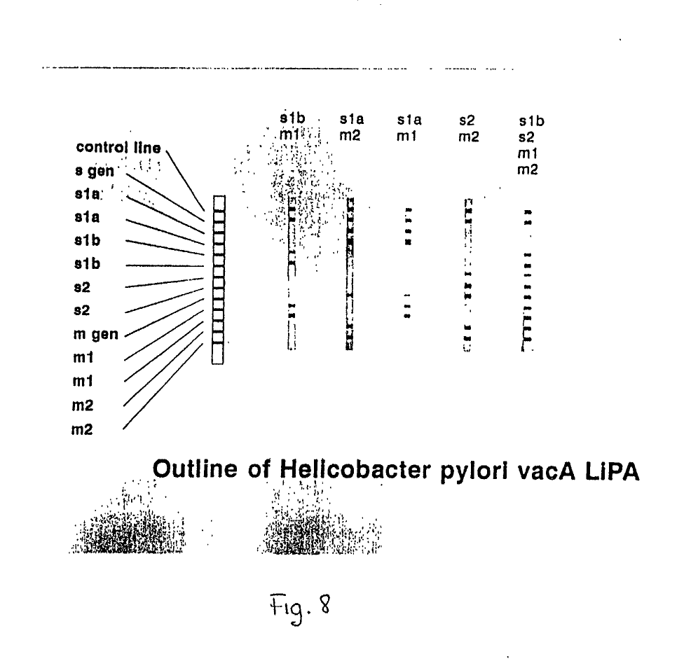

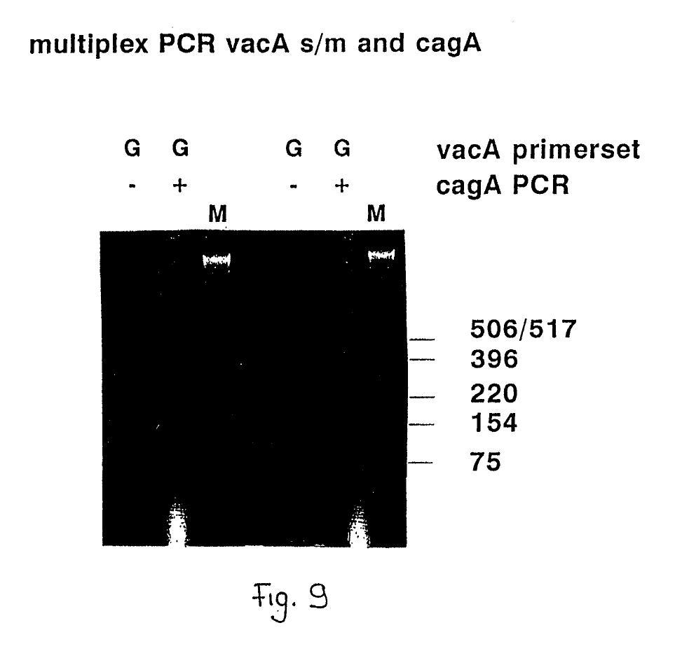

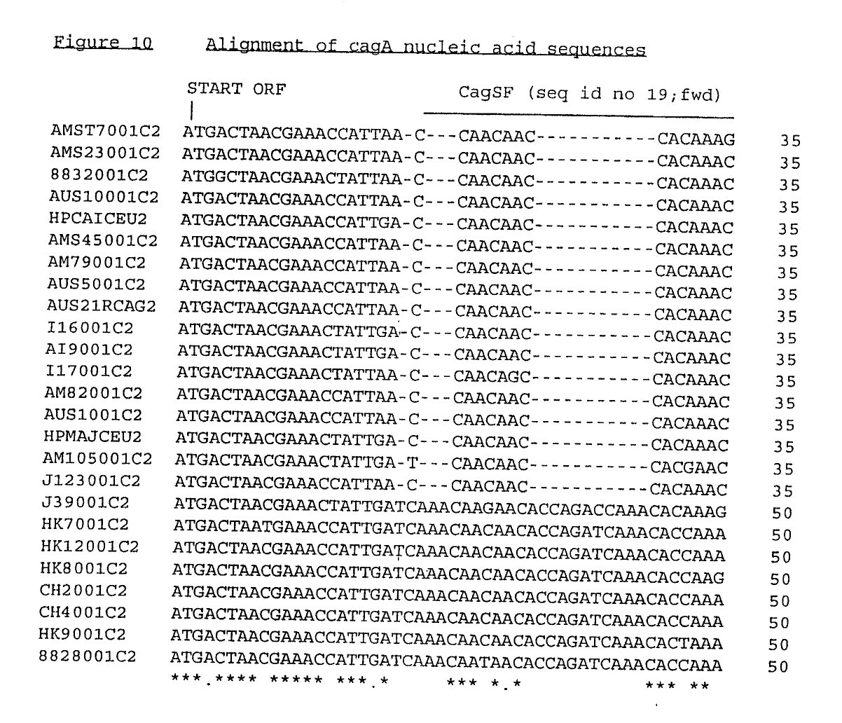

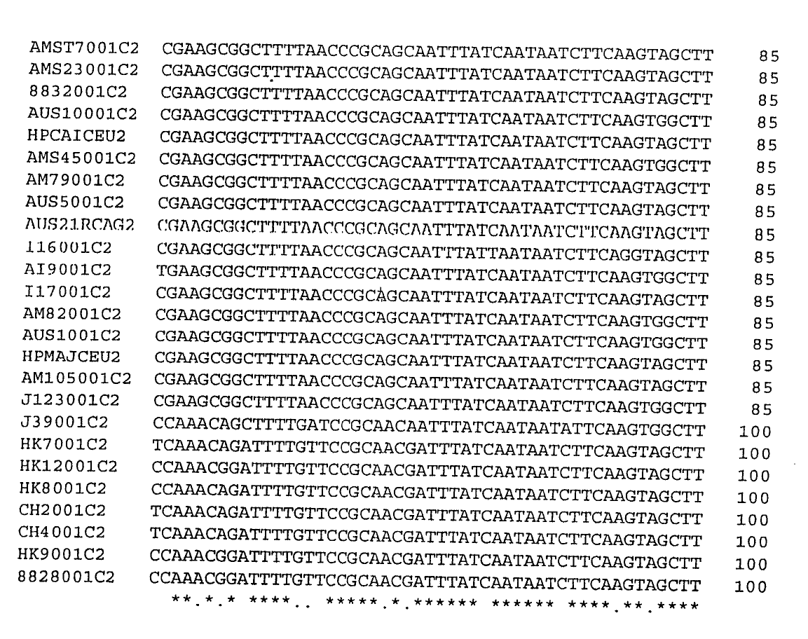

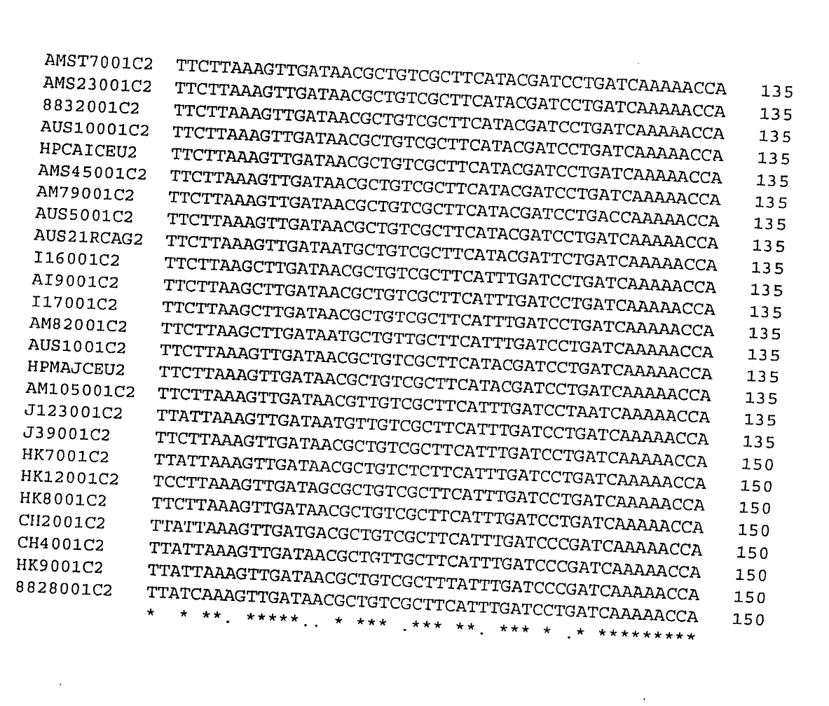

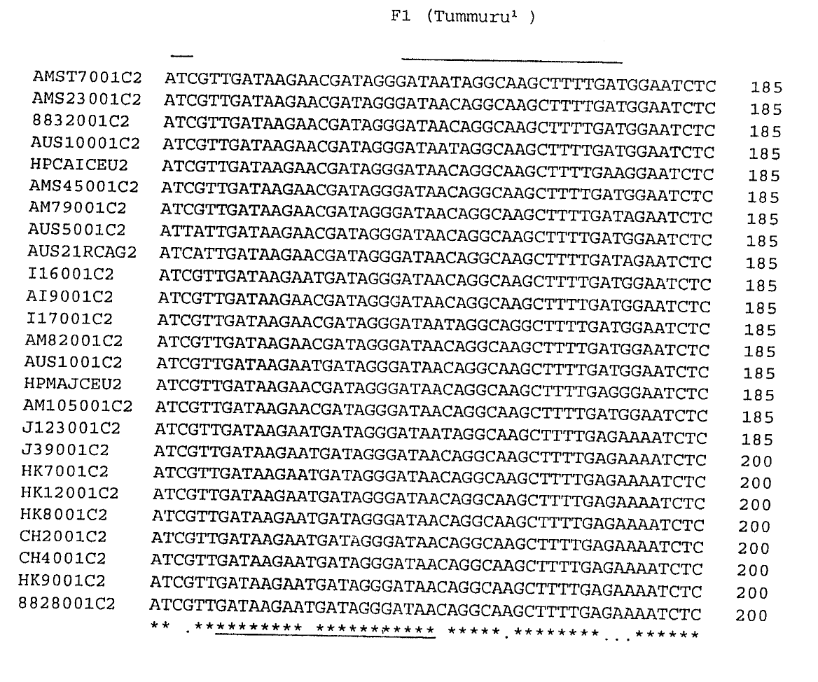

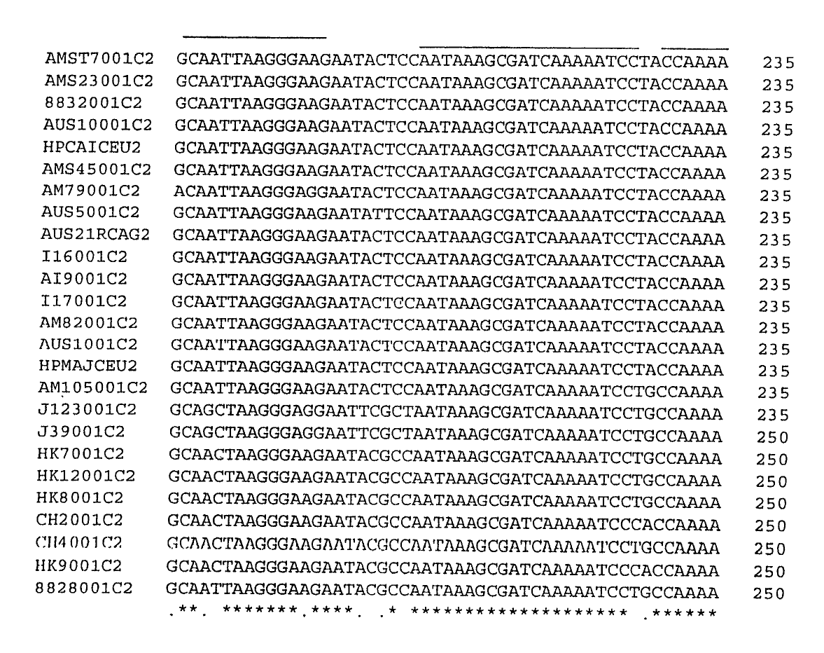

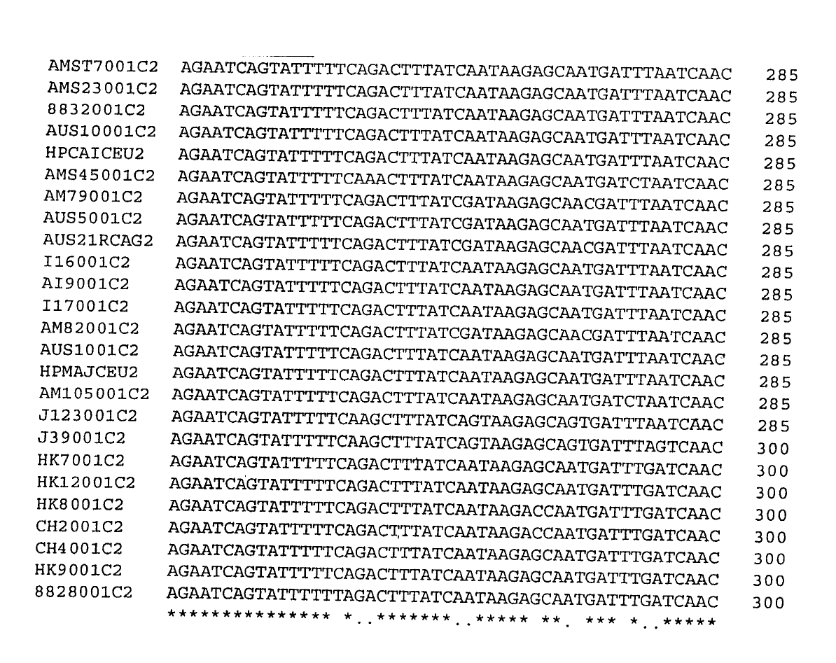

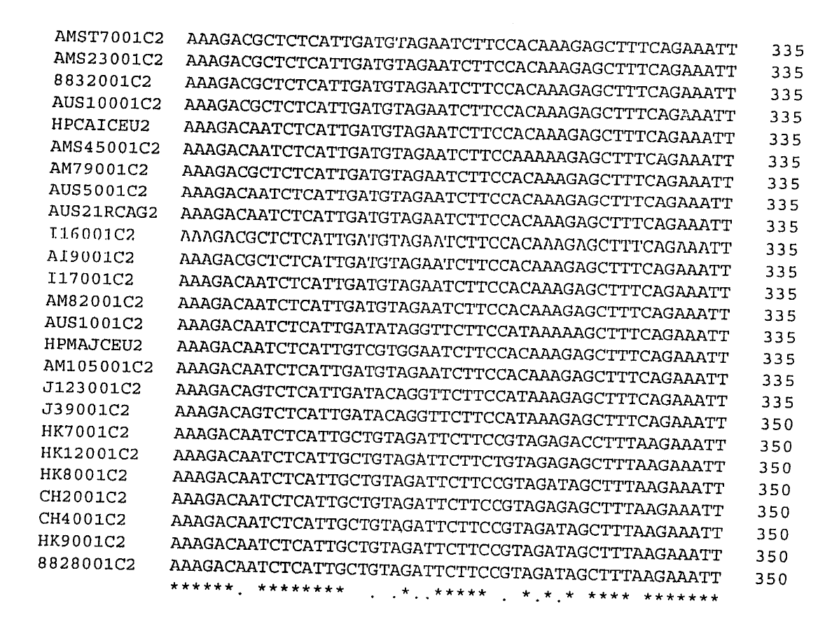

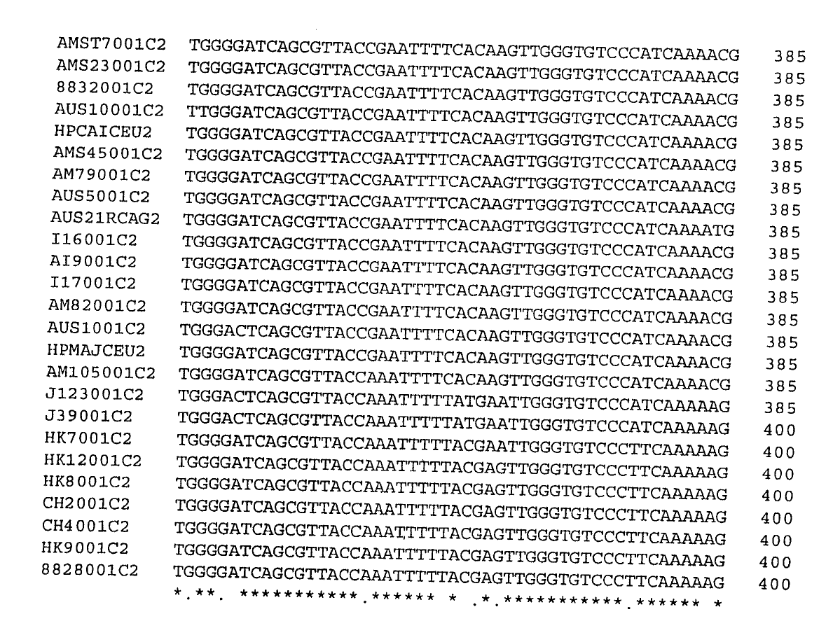

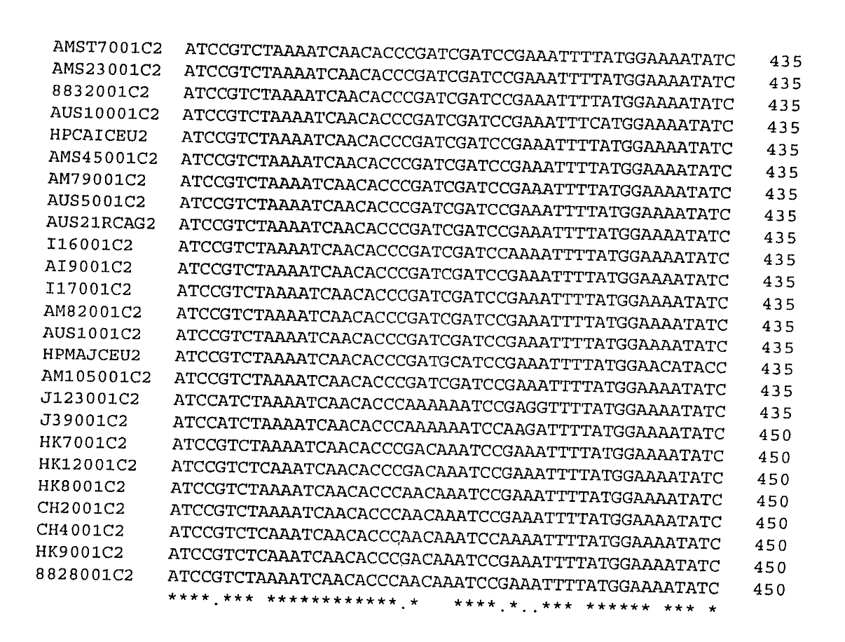

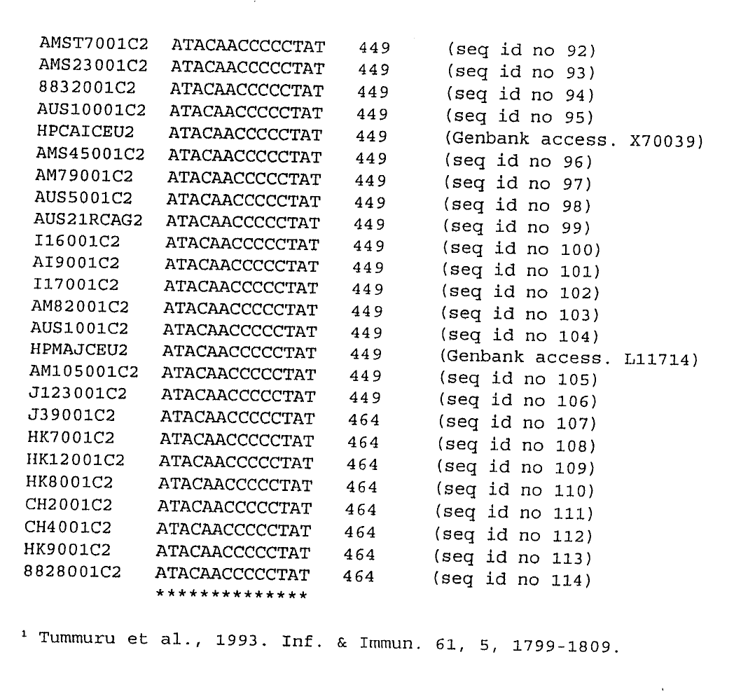

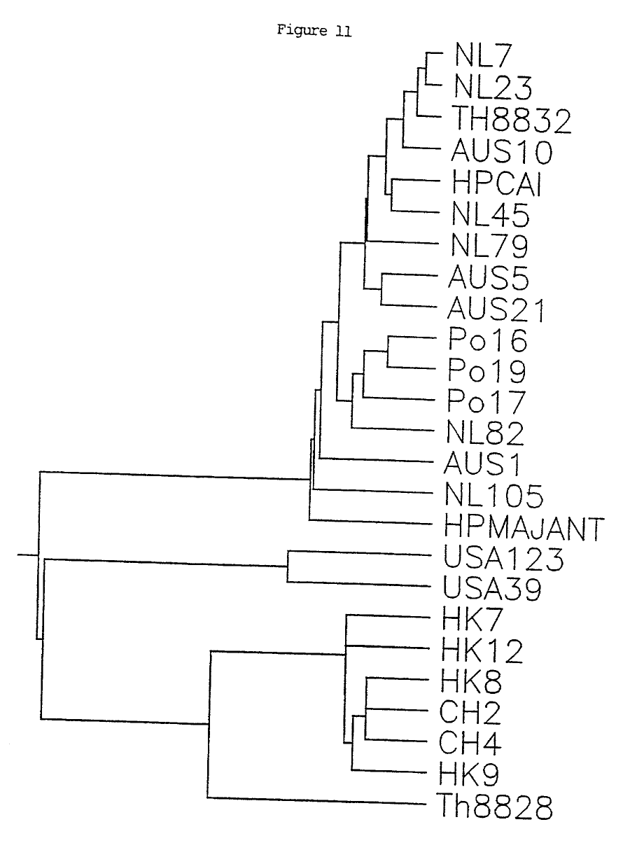

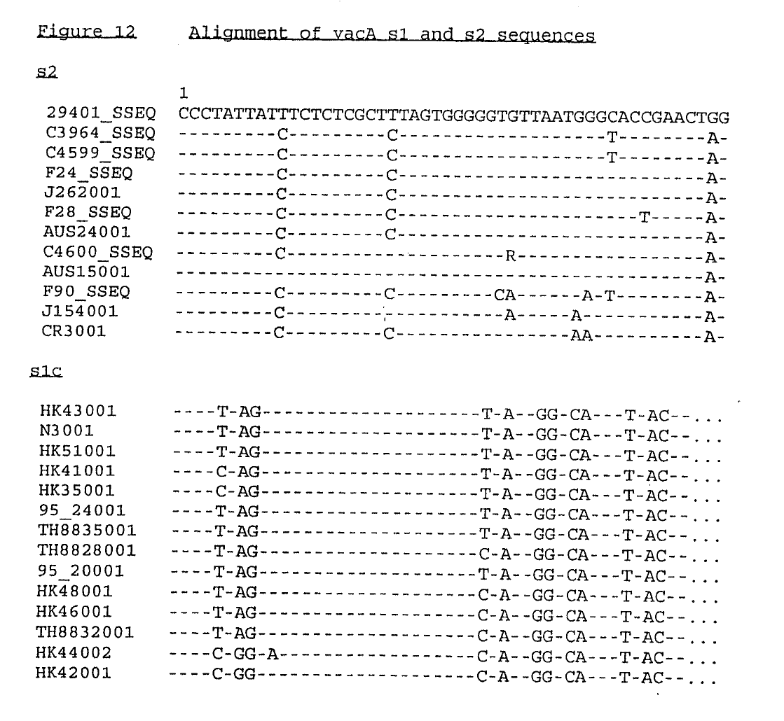

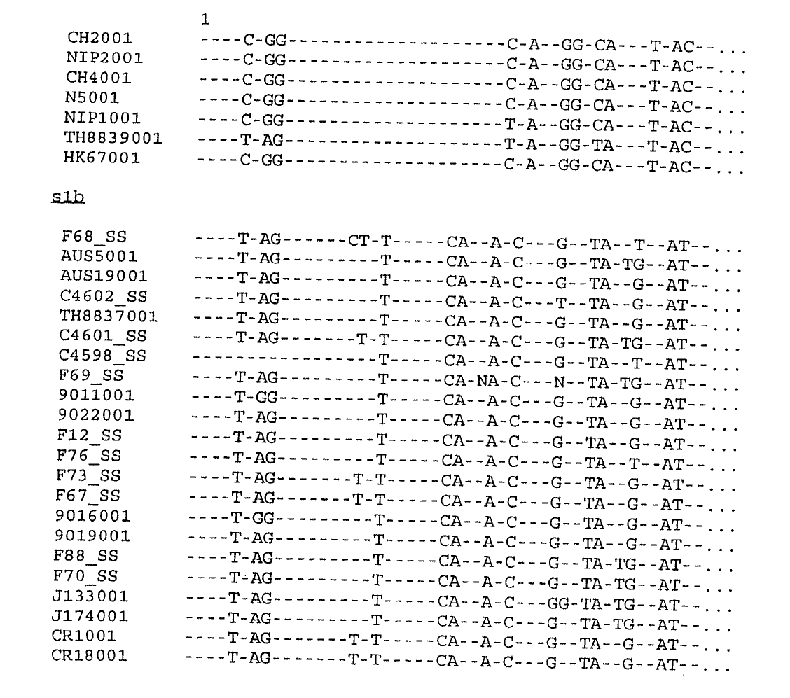

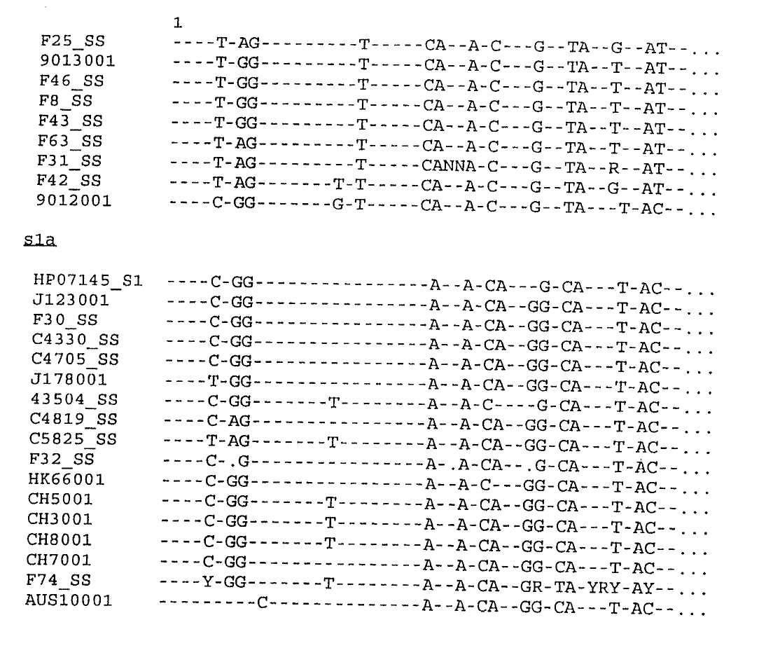

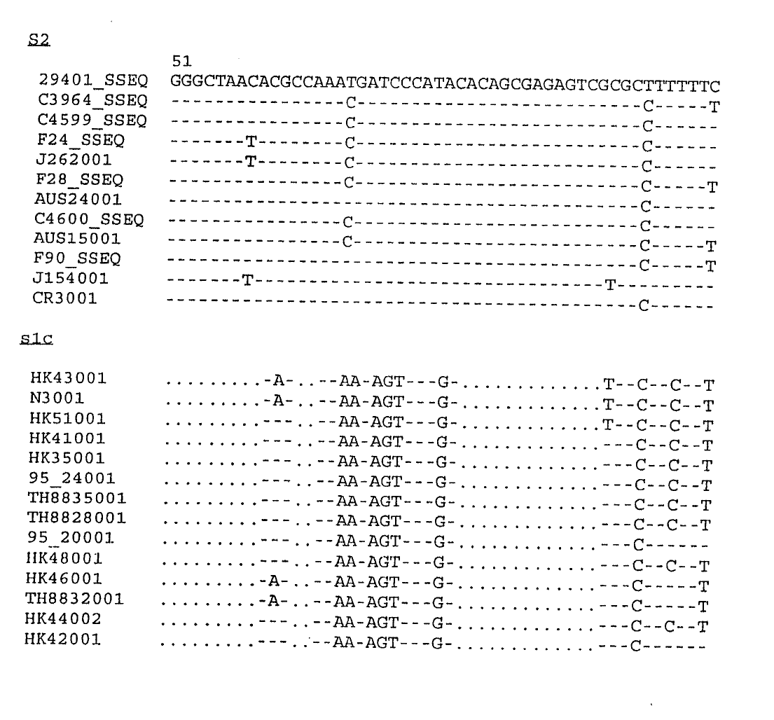

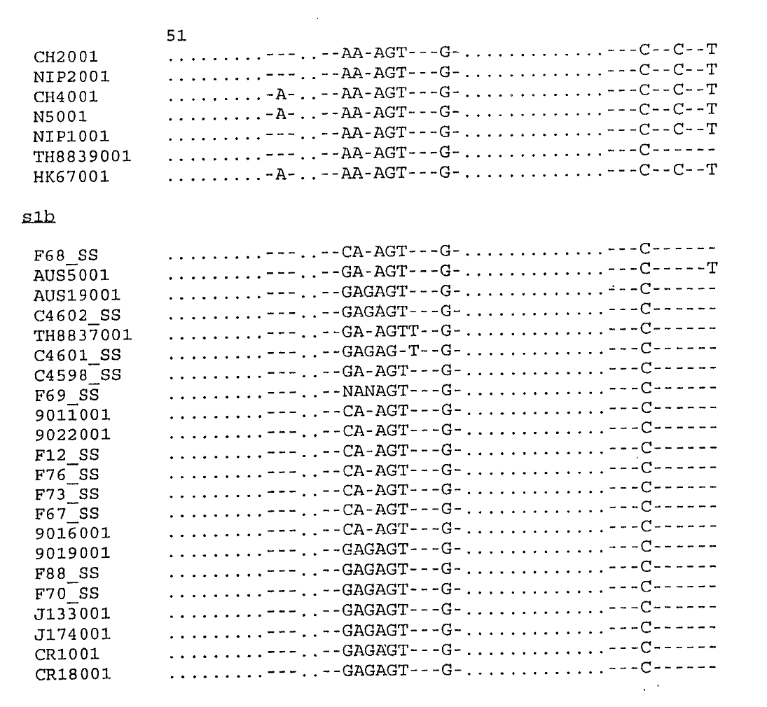

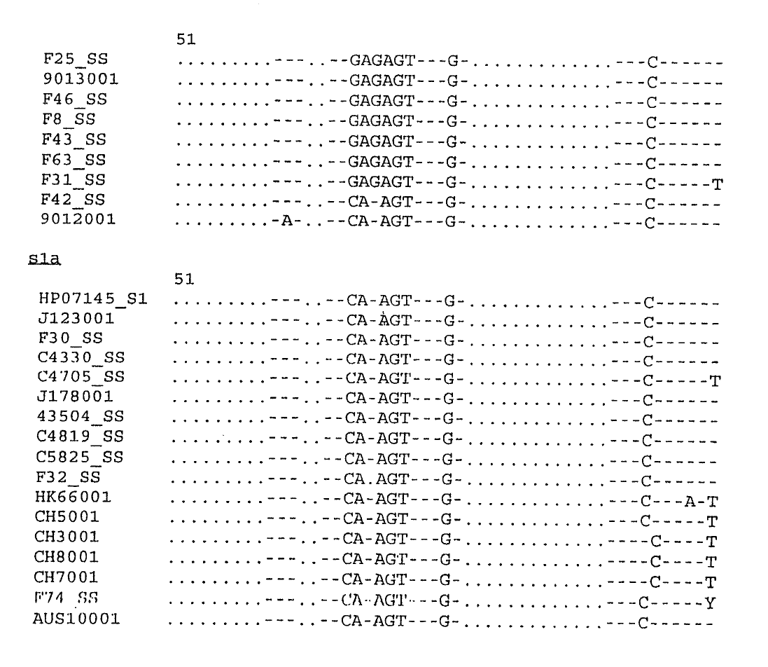

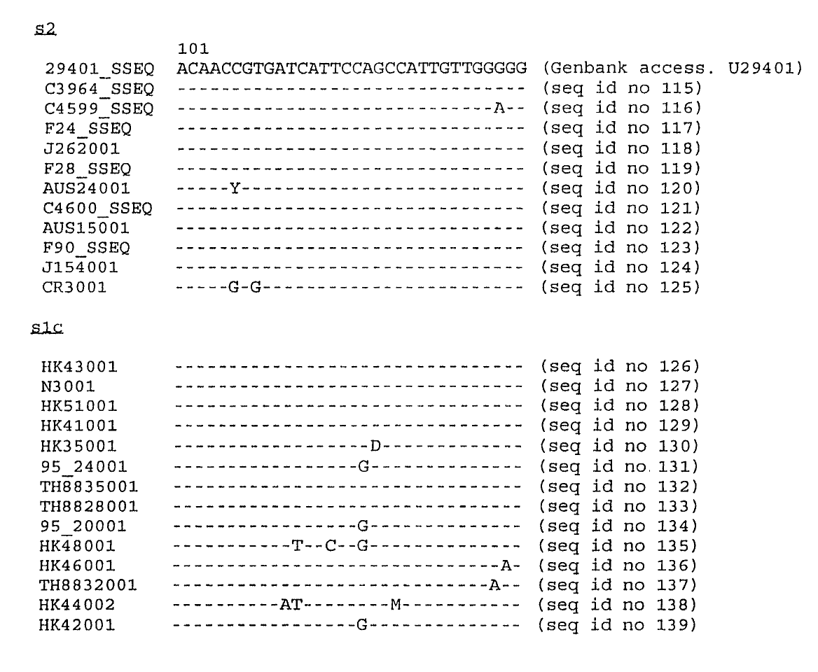

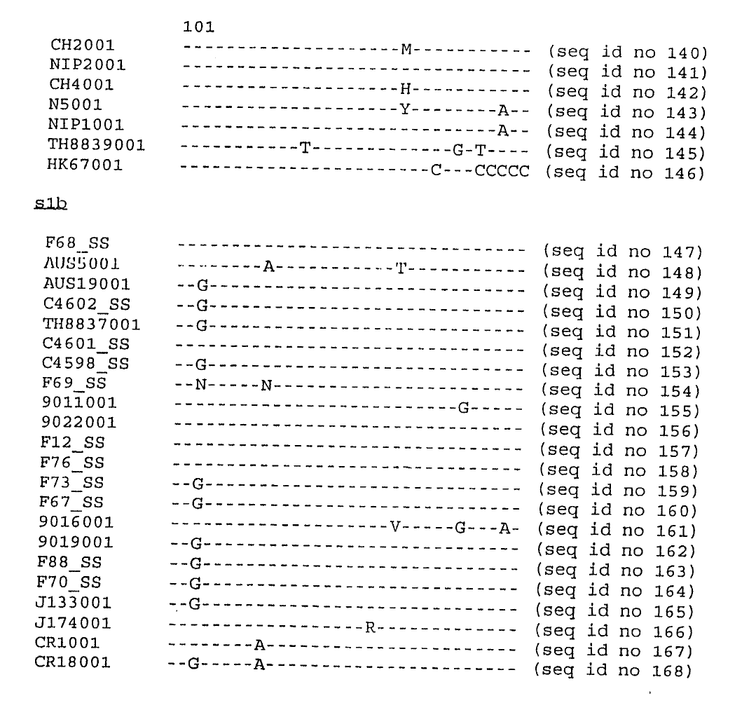

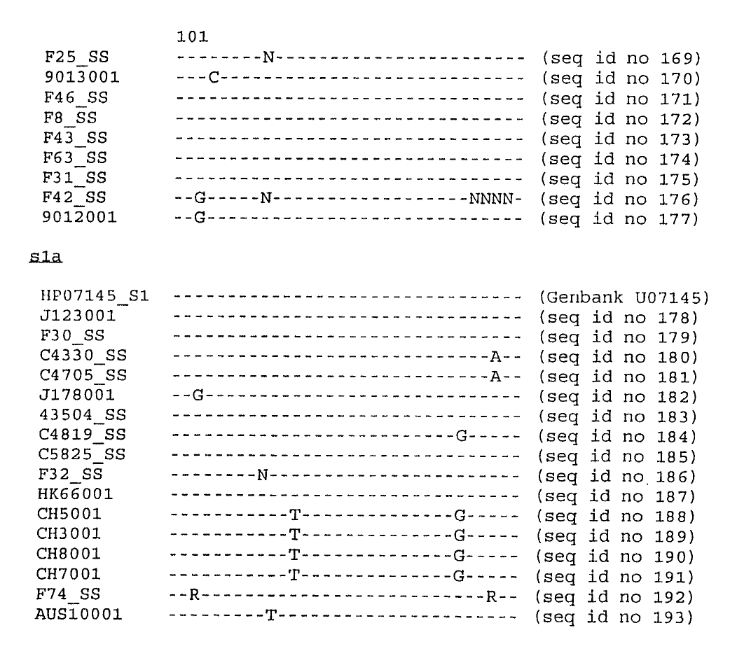

Amplification products were analysed by DNA sequencing applying standard sequencing techniques. The results of these analyses are given in FIGS. 2 and 3. Based on these analyses, it became obvious that primers being used by others with the aim of allele-specific typing of H.pylori based upon the variable S- and M-region of the vacA gene, could not cover the full range of pathogenic H.pylori strains (see example 1). Thus, new sets of primers, not obvious to the skilled man in the art, were designed in order to develop an assay to detect and type pathogenic H.pylori strains in a sample. The primers and their sequence are given in table I. Also, a number of probes were tested in order to obtain optimal hybridization between the amplified products, generated by the new primer sets, and said probes under standardized hybridization and washing conditions applied in the reverse hybridisation assay. The tested probes are given in table II. Said probes were immobilized unto a solid support as described by Styver et al., 1993. The amplification with said primersets was performed under the conditions and protocol as described above in this example. The amplified products obtained with said above primers were hybridized to the respective probes (see FIG. 8).

Optimal results were obtained combining the following primers:

| | |
|---|---|
| VA1-F | (Atherton et al., 1995) |
| VA1XR | (SEQ ID N014) |
| M1F | (SEQ ID NO15) |
| M1R | (SEQ ID N016) |

with the following probes:

| | |
|---|---|
| P1S1 | (SEQ ID NO2) |
| P22S1a | (SEQ ID NO3) |
| P1S1b | (SEQ ID NO4) |
| P2S1b | (SEQ ID NO5) |
| P1S2(VAS2) | (SEQ ID NO6) |
| P2S2 | (SEQ ID NO7) |
| P1M1 | (SEQ ID NO8) |
| P2M1 | (SEQ ID NO9) |
| P1M2 | (SEQ ID NO10) |
| P2M2 | (SEQ ID NO11) |

### Example 4

### Development of the Line Probe Assay (LiPA)-Strip

The principle and protocol of the line probe assay was in essence as described earlier (Stuyver et al., 1993). Good results were obtained combining the following primers:

| | |
|---|---|
| cagF | (SEQ ID NO 12) |
| cagR | (SEQ ID N0 13) |
| VA1-F | (Atheron et al., 1995) |
| VA1XR | (SEQ ID NO 14) |
| M1F | (SEQ ID NO 15) |
| M1R | (SEQ ID NO 16) |

with the following probes:

| | |
|---|---|
| cagApro | (SEQ ID NO 1) |
| P1S1 | (SEQ ID NO 2) |
| P22S1a | (SEQ ID NO 3) |
| P1S1b | (SEQ ID NO 4) |
| P2S2b | (SEQ ID NO 5) |
| P1S2(VAS2) | (SEQ ID NO 6) |
| P2S2 | (SEQ ID NO 7) |
| P1M1 | (SEQ ID NO 8) |
| P2M1 | (SEQ ID NO 9) |
| P1M2 | (SEQ ID NO 10) |
| P2M2 | (SEQ ID NO 11) |

The said primers were labeled with biotine. Different primer-based target-amplfication systems may be used. For amplification using the PCR, the conditions used in case of a single-round amplification with above primers, involve 40 cycles of 1 min/95° C., 1 min/55° C., 1 min/72° C. followed by a final extension for 5 min at 72° C.

The PCR reaction mixture was as follows:

1 μl DNA sample, containing H.pylori or control DNA

10 μl 10×polymerase mix (final concentration 10 mM Tris HCl, pH 9.0, 50 mM KCl 2.5 mM MgCl₂, 0.01% gelatin and 0.1% Triton)

20 μl deoxyribonucleotide mix (final concentration 200 μM each)

1 μl Super Taq polymerase (0.25 U/μl)

1 μl forward primer (50 pmoles/μl)

1 μl reverse primer (50 pmoles/μl)

66 μl water

100 μl

The sequence of these primers is given in table 2. An example of the amplification products generated by use of vacA s/m region-primers or cagA-primers is shown in FIG. 9. For this experiment primer set G (FIG. 1) was used for vacA and cagf and cagr were used for cagA. The isolate shown in the first two lanes contains s1 and m1 alleles and is cagA+. The isolate shown in lanes 4 and 5 (counting from left) contains a multiple infection. The results of the LiPA are shown in FIG. 8.

### Example 5

### Novel DNA Sequences of a Fragment of the cagA Gene of H. pylori and Design of Primers and a Probe Based Thereon.

The 5′ part of the cagA gene was amplified by PCR from various H. pylori isolates, using different primer combinations. The resulting fragments were sequenced and the alignment is shown in FIG. 10. The sequences comprised 449-464 bp, starting at the start codon of the ORF. A total of 149-154 amino acids representing the N-terminus of the cagA protein can be derived by translation of these sequences, sting at the ATG codon at position 1 in FIG. 10.

As shown by phylogenetic analysis in FIG. 11, 2 different forms of cagA were recognized. The first variant is highly homologous to the reference sequence (Genbank accession number L11741 (HECMAJANT) or X70039 (HPCAI)) and occurs mainly in strains from Europe and Australia. Two sequences from the USA (J123 and J39) seem to have intermediate positions in the phylogenetic tree. The second variant, mainly found in strains from Far East Asia. contains 15 additional nucleotides between nt positions 20 and 31, encoding 5 additional amino acids between positions 8-9, as compared to the reference sequence.

From the nucleotide sequence alignment the following novel primers and probe were deduced aimed at highly conserved regions in the cagA gene.

TABLE 3
CagA primers and probe

| | | position/ |
|---|---|---|
| primer/probe | 5′to 3′sequence | orientation¹ |
| primers | | |
| cagFN1 | GATAAGAAYGATAGGGATAA | +(142-161) |
| cagRN1 | AATACTGATTCTTTTTGG | −(230-247) |
| probe | | |
| cagprobe3 | GGATTTTTGATCGCTTTATT | −(219-227) |

¹Positions according to the ATG of the ORF at position 1.

### Example 6

### Novel DNA Sequences of the s-Region of the vacA Gene of H. pylori and Design of Probes Based Thereon.

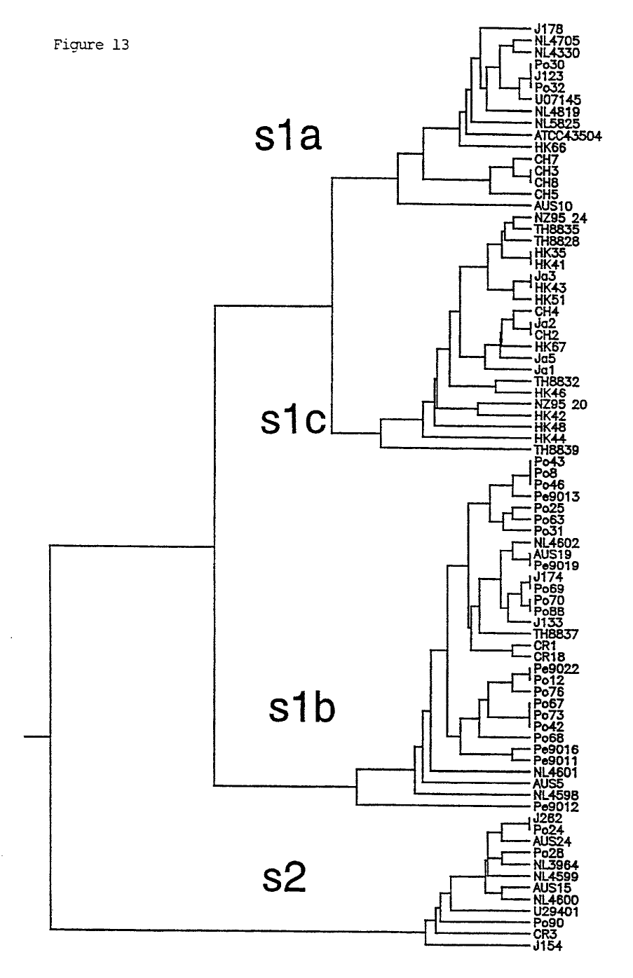

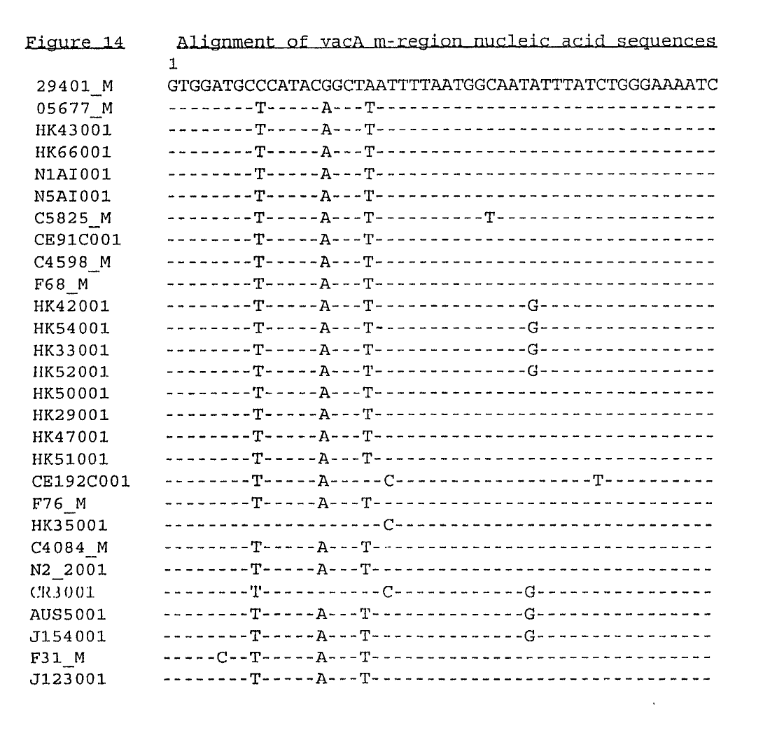

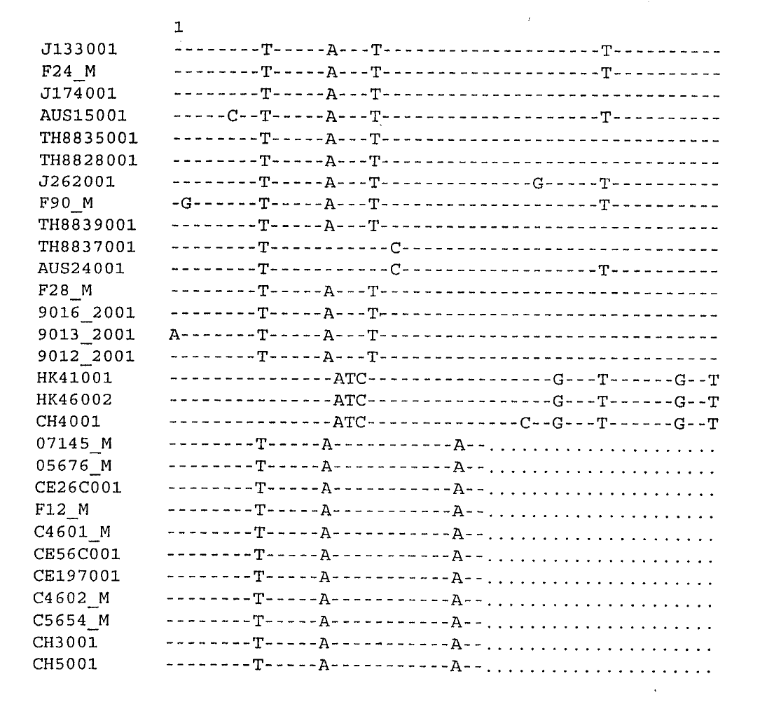

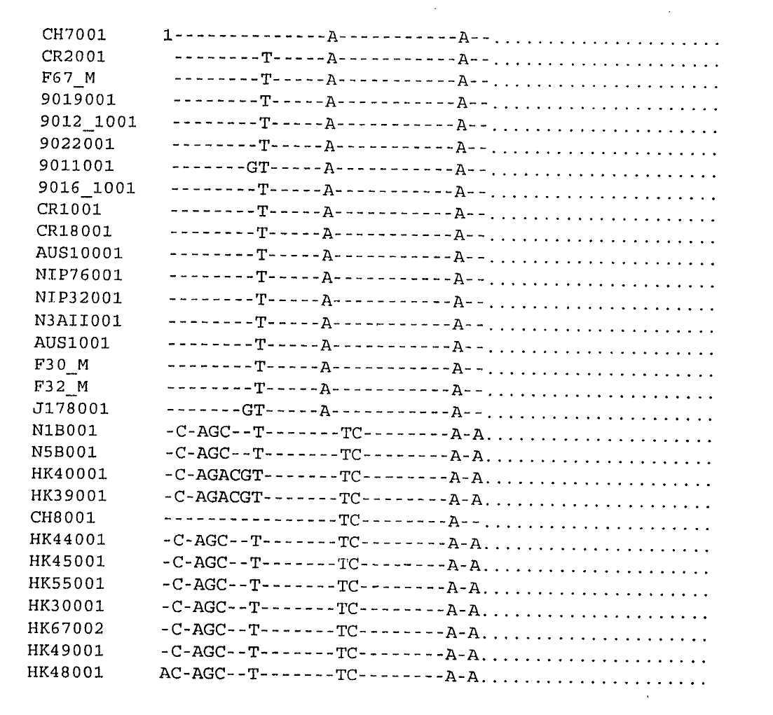

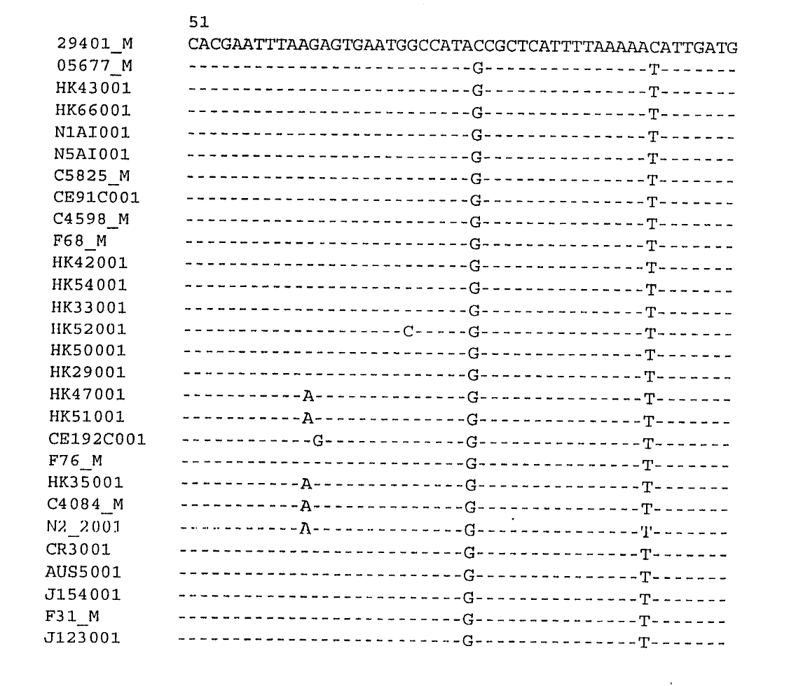

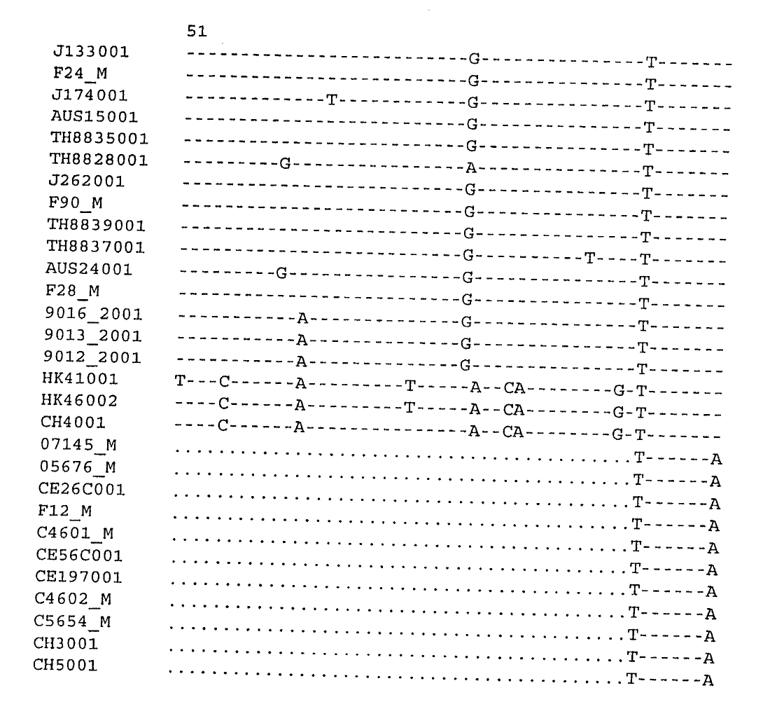

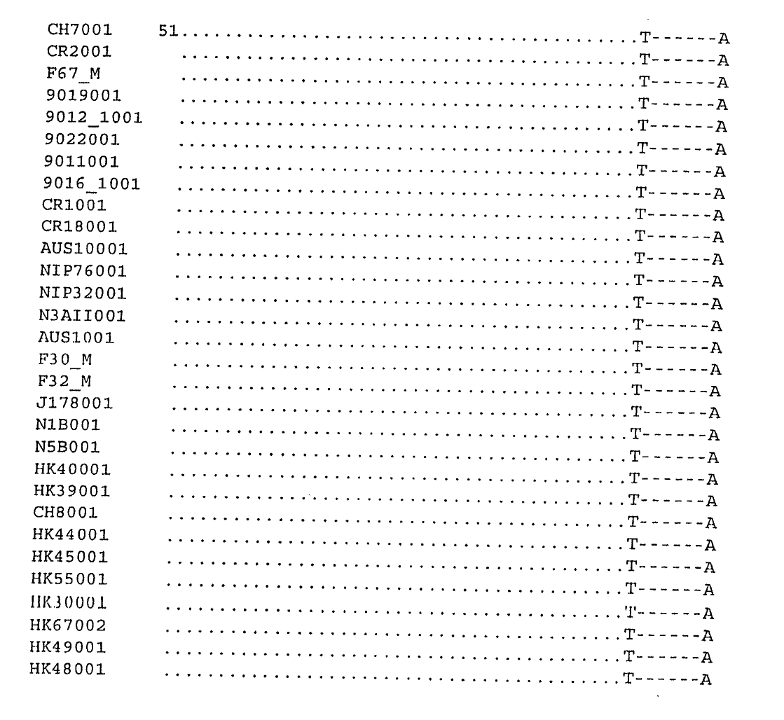

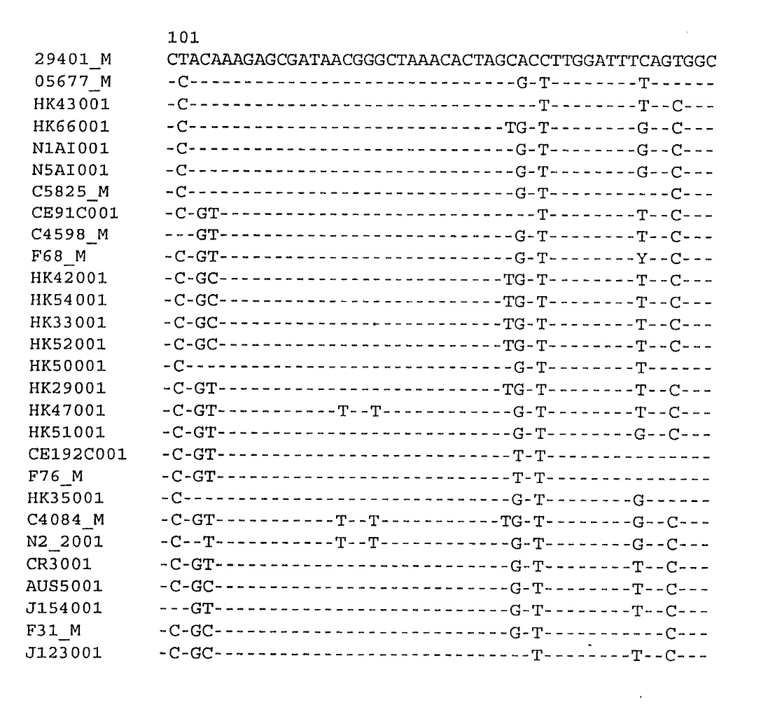

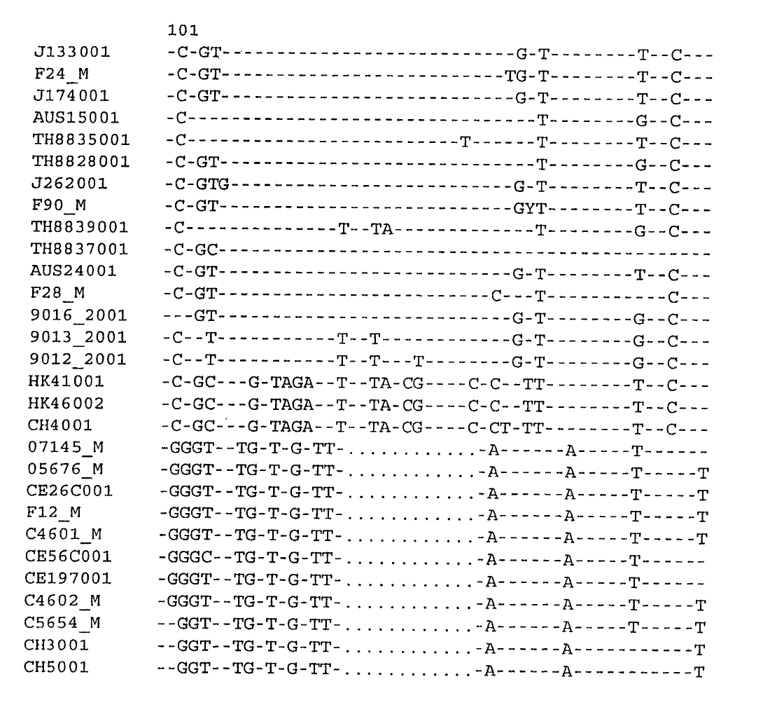

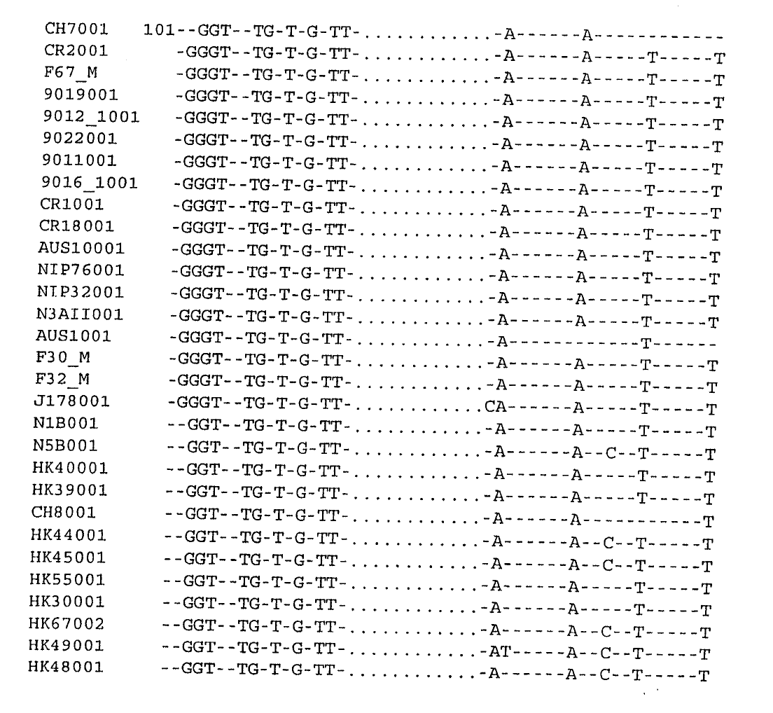

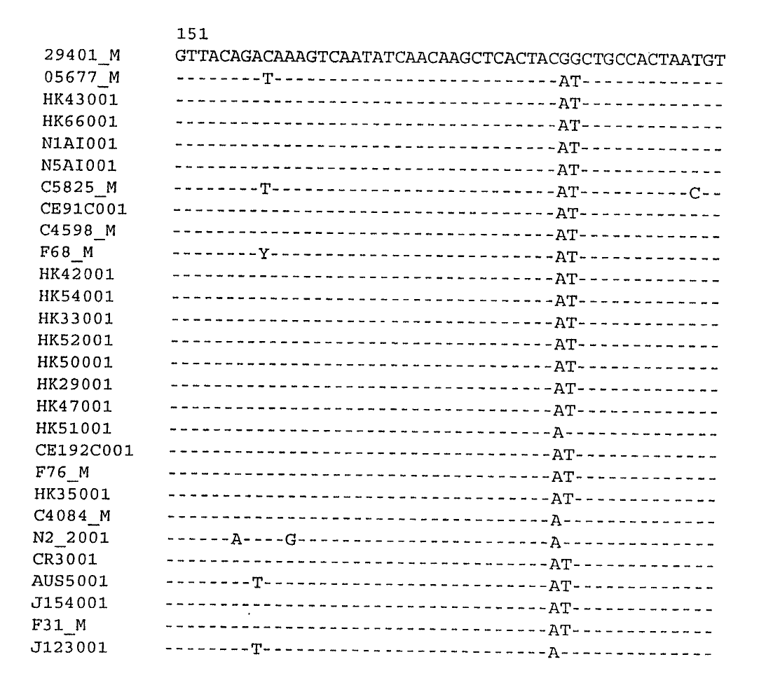

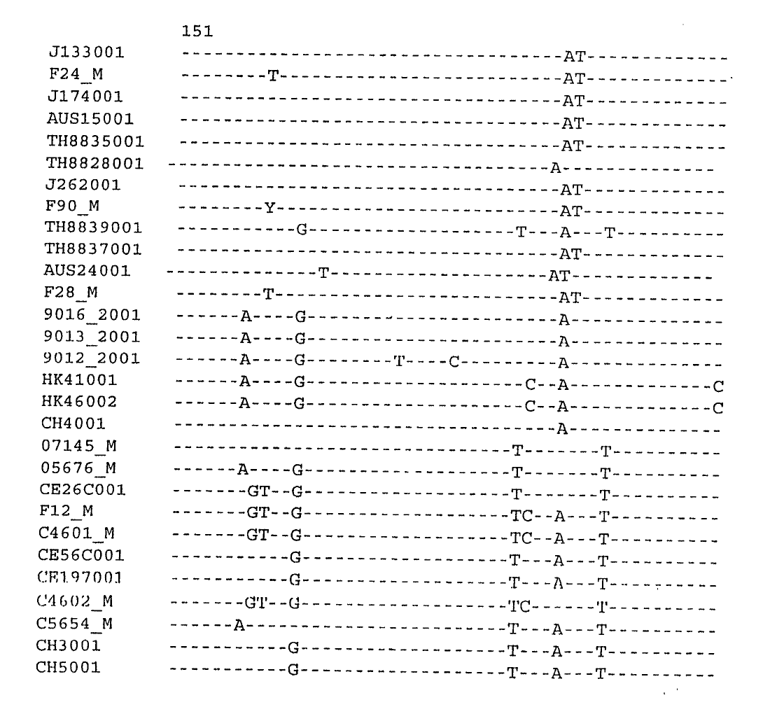

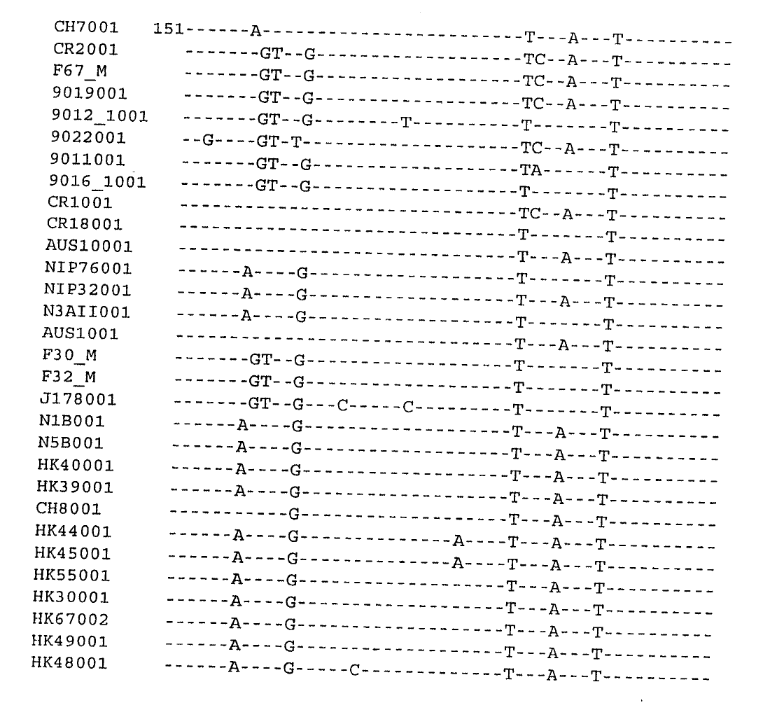

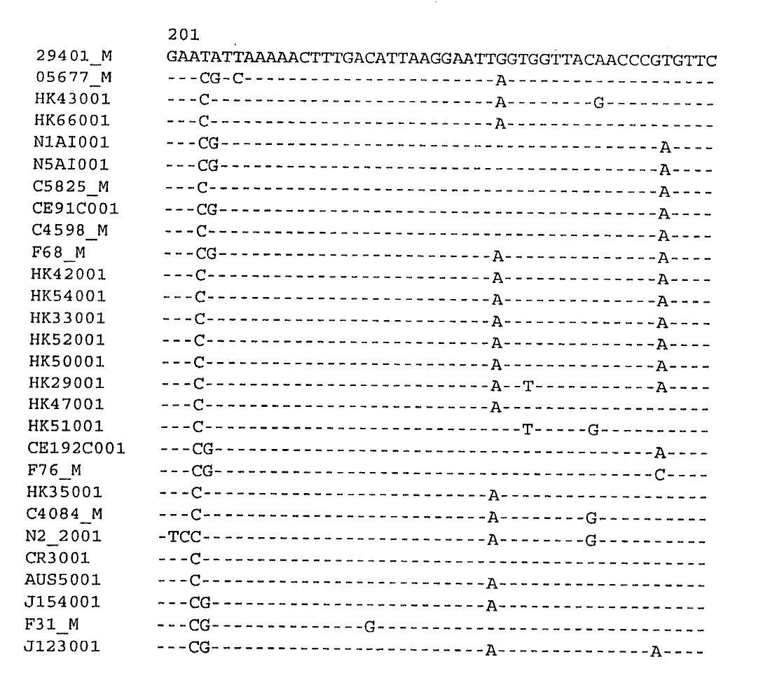

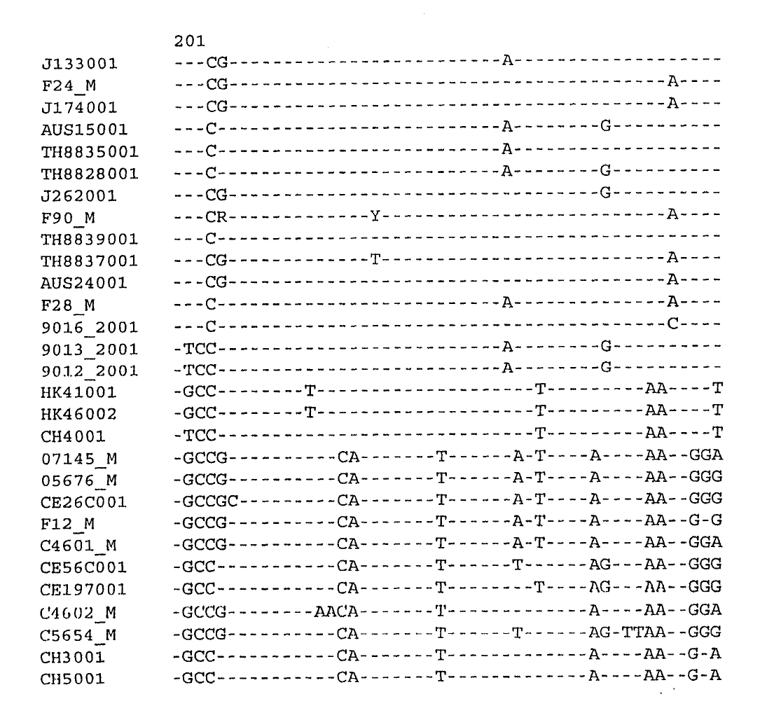

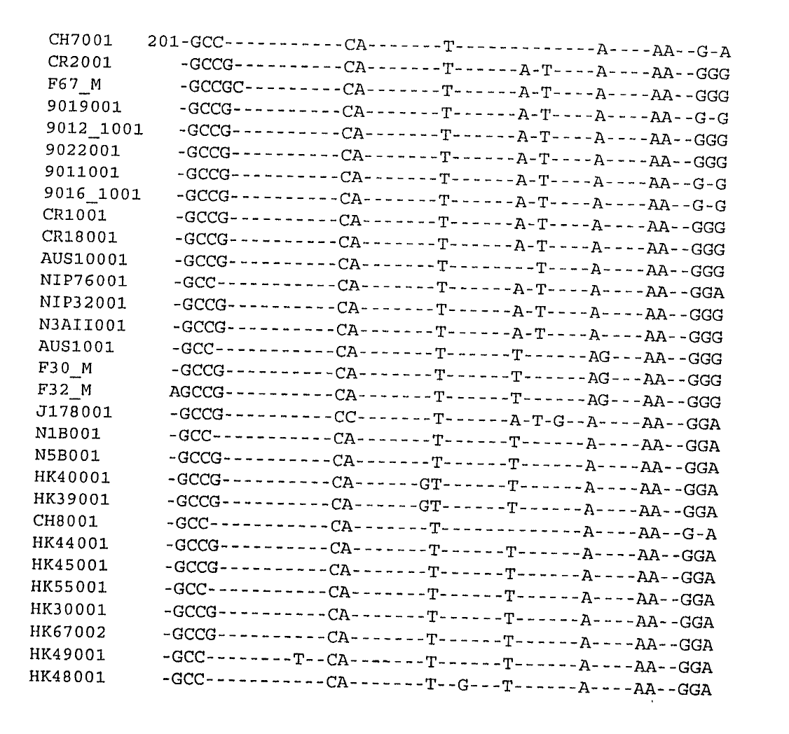

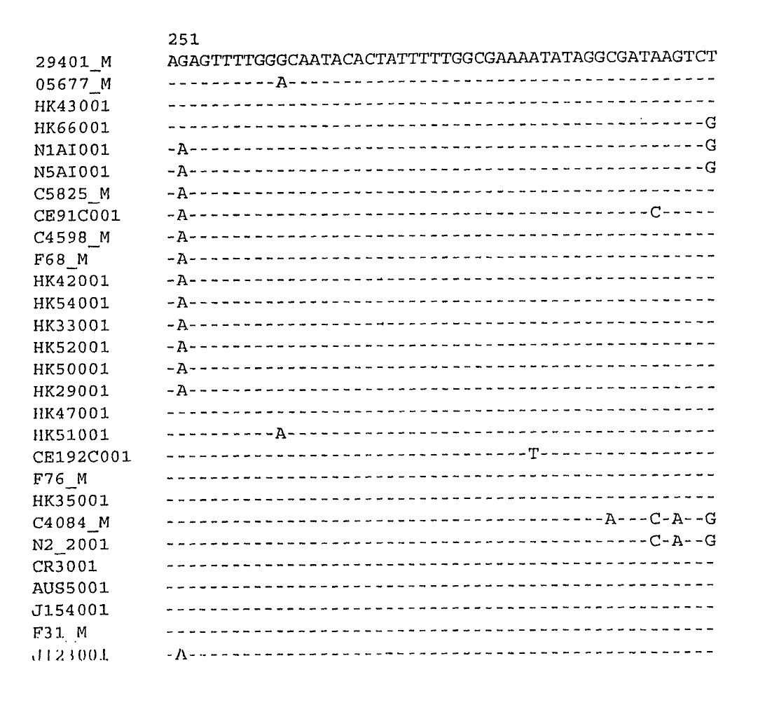

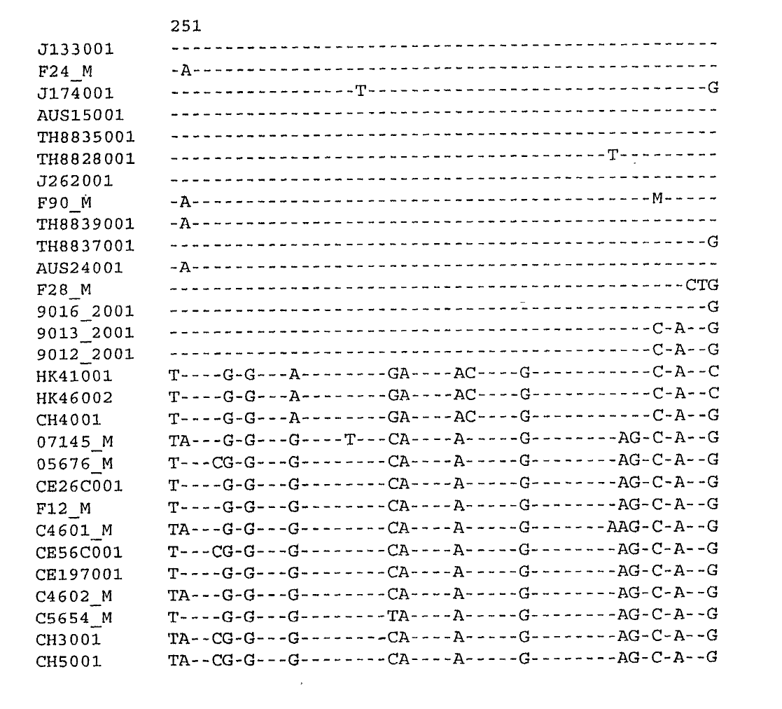

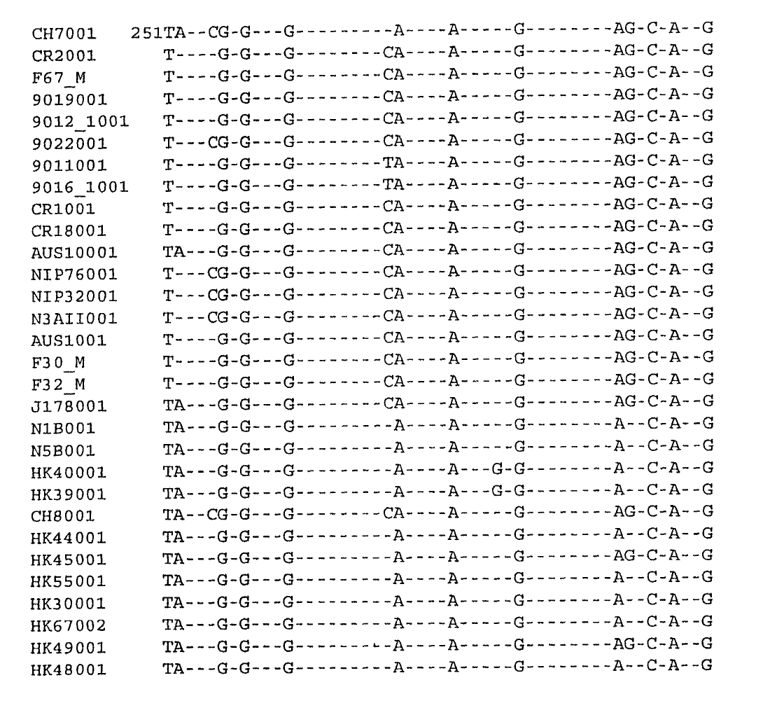

VacA s-region fragments were amplified from a large number of H. pylori isolates, using primers VA1-F and VA1-R (Atherton et al., 1995). This resulted in fragments of 176 bp for s1 and 203 bp for s2 types sequences. Parts of these fragments were sequenced, and the resulting alignment of 80 sequences (including 2 reference sequences U29401 and U07145) is shown m FIG. 12. Apart from the already known s1a and s1b type sequences, a third variant was observed, mainly in isolates from Far East Asia (Japan, China. Hong Kong), This variant is designated s1c. Type s1c has several highly consistent mutations as compared to type s1b and s1a. These mutations allow specific recognition of each of the s1 subtypes. Phylogenetic analysis, as shown in FIG. 13, reveals distinct clusters of s1a, s1b, s1c and s2 sequences. The N-terminal parts of the vacA protein can be deduced from the nucleic acid sequences of the s1a, s1b, s1c, and s2 variants by translation starting at codon CCT at position 2 in FIG. 12. This reveals the presence of a single conserved amino acid imitation (Lys) at position 22 in subtype s1c as compared to s1a and/or s1b sequences. All other nucleotide mutations appear to be silent.

New probes were designed to specifically detect the s1c variants:

| | |
|---|---|
| P3s1: 5′ GGGYTATTGGTYAGCATCAC 3′ | (positions 26-45) |
| P4s1: 5′ GCTTTAGTRGGGYTATTGGT 3′ | (positions 17-36) |

Thus, for optimal detection of the vacA s-region variants, the following probes were used:

| | |
|---|---|
| for s1a: | P1S1 and P22S1a |
| for s1b: | P1s1b and P2s1b |
| for s2: | P1S2 (VAS2) and P2S2 |
| for s1c: | P3s1 and P4s1 |

### Example 7

### Novel DNA Sequences of the m-region of the vacA Gene of H. pylori and Design of Probes Based Thereon.

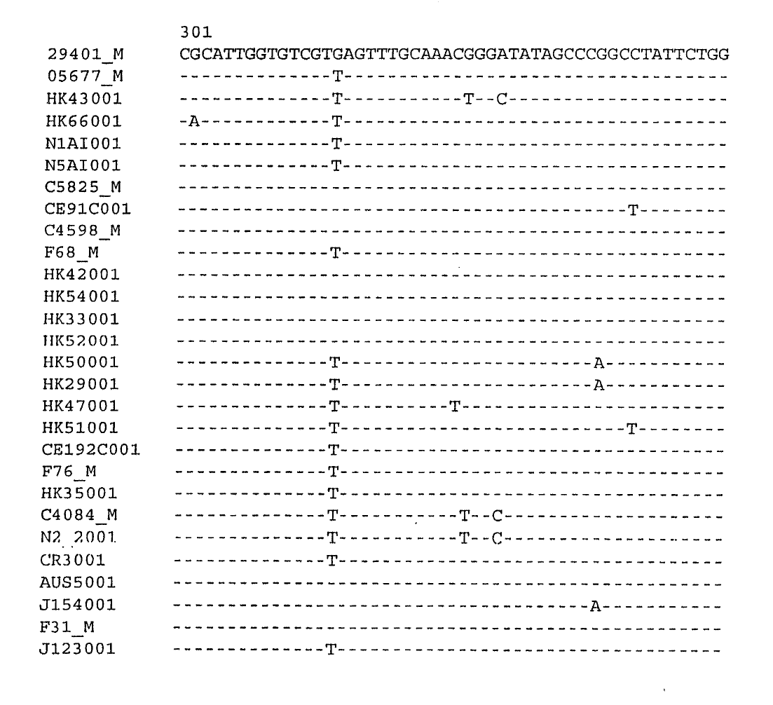

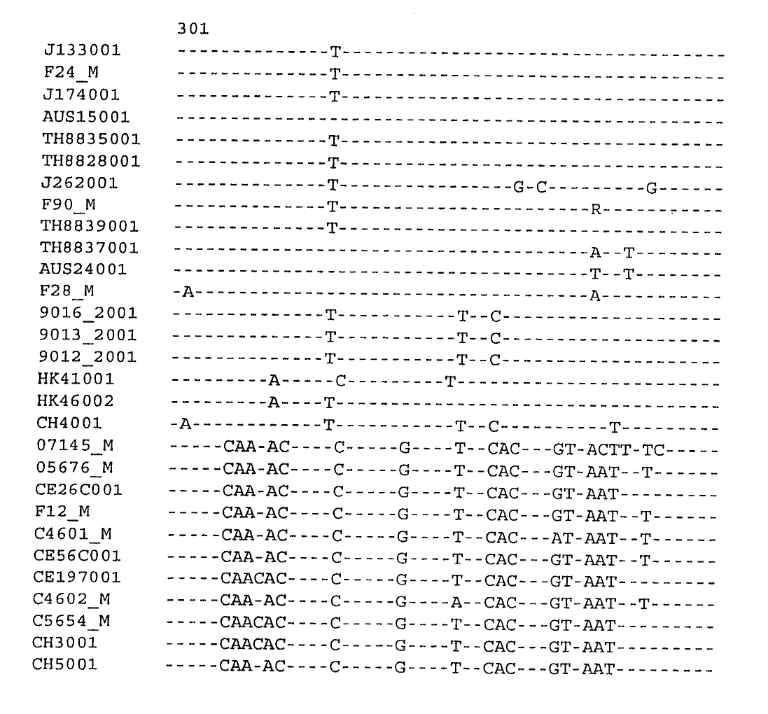

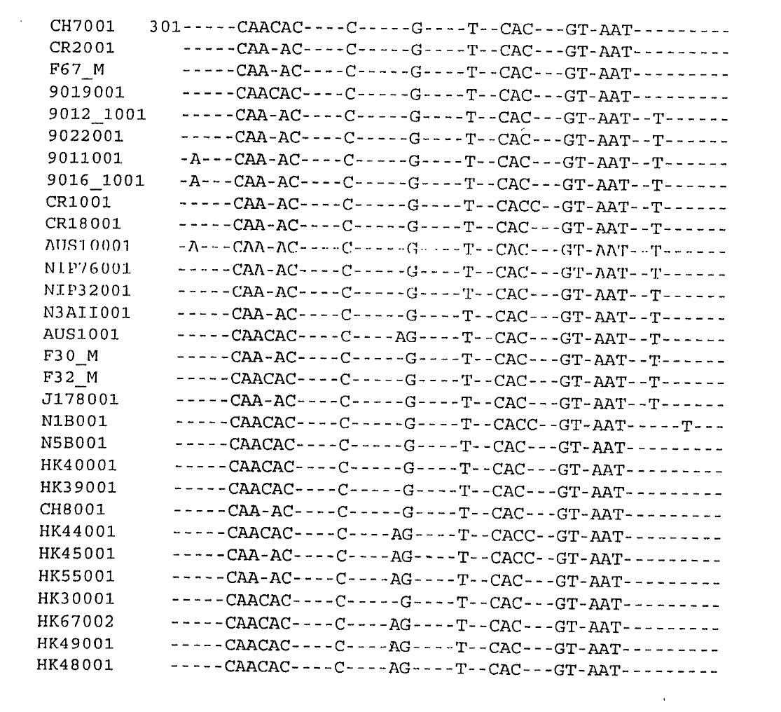

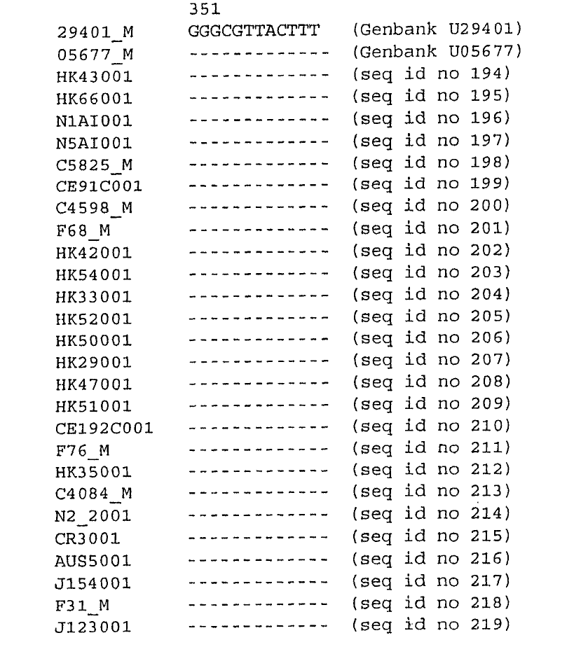

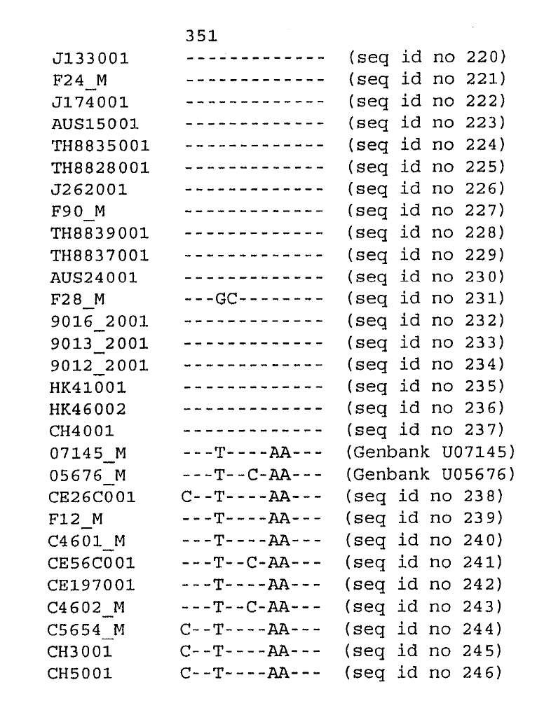

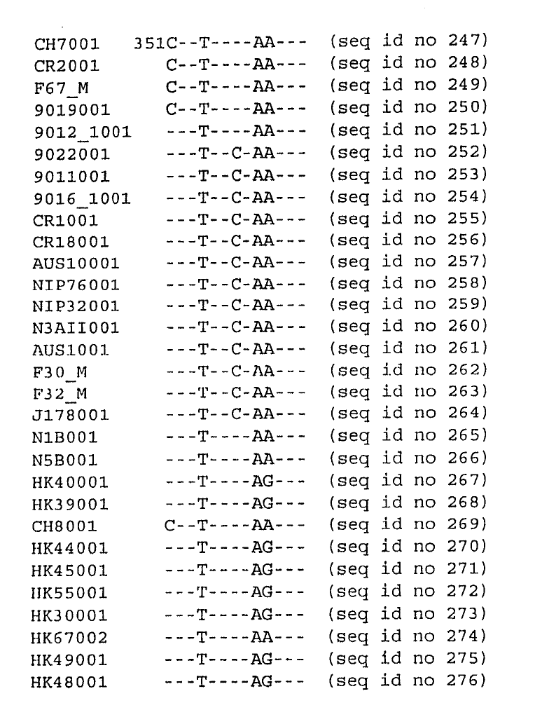

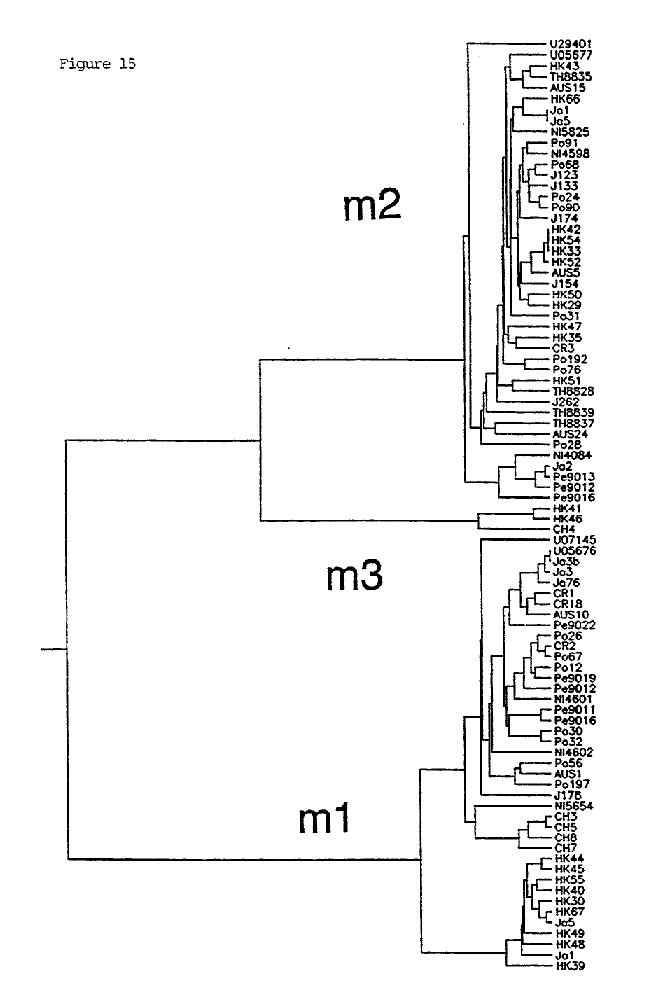

The vacA m-region was analyzed from a number of H. pylori isolates, by using primers BPMGF and HPMGR These primers allow general amplification of larger parts of the m-region sequences and generate fragments of 401 and 476 bp for m1 and m2 variants, respectively. Fragments were sequenced and the alignment of 86 m-region sequences (including reference sequences U05677, U07145, U05676 and U29401) is shown in FIG. 14. The phylogenetic tree is shown in FIG. 15. The alignment revealed the presence of 3 sequences (Ch4, Hk41, Hk46) that are different from the published m1 and m2 variants. These sequences nay represent another variant in the m-region. Said new variant may be denoted m3.

These alignments revealed that the target sequence for forward primer M1F (SEQ ID NO15) was not completely conserved among all isolates. The target sequence for reverse primer M1R appeared highly conserved among all isolates. As an alternative for forward primer M1F the following primers were designed, as shown in table 4.

TABLE 4
Novel forward primers for the vacA m-region

| primer | sequence 5′to 3′ | orientation |
|---|---|---|
| VAMSFb: | GTGGATGCCCATACGOCTAA | forward |
| VAMSFc | GTGGATGCTCATACAGCTWA | forward |
| VAMSFd | GTGGATGCCCATACGATCAA | forward |
| VAMSFe | GCGAGCGCTCATACGGTCAA | forward |

PCR amplification in the m-region of the vacA gene can thus be performed by use of VAMSFb,c,d, and e as forward primers and M1R as the reverse primer.

Novel probes were designed for specific hybridization to m1 and m2 variants. Their sequence is based on the above-mentioned probes P1m1, P2m1, P1m2 and P2 m2. In order to obtain reactivity with all sequences, a few degeneracies were included. The novel sequences are shown in table 5. For specific identification of mn3 variants, a single probe is added (P1m3).

TABLE 5
Novel probes for the vacA m-region

| probe | sequence 5′-3′ | positions |
|---|---|---|
| P1m1new | TTGATACKGGTAATGGTGG | as for P1m1 |
| P2m1new | KGGTAATGGTGGTTTCAACA | as for P2m1 |
| P1m2new | KGGTAATGGTGGTTTCAACA | as for P1m2 |
| P2m2new | AGAGCGATAAYGGKCTAAACA | as for P2m2 |
| P1m3 | AGGGTAGAAATGOTATCGACA | 1577-1597¹ |

¹The position of probe P1m3 is identical to the position of P1m2 and P2m2, although there is no reference sequence for this m-type available in the Genbank.

### Example 8

### Detection of H. pylori DNA by PCR and DNA Enzyme Immuno Assay (DEIA).

This method is used for rapid and specific detection of PCR products. PCR products are generated by a primerset, of which either the forward or the reverse primer contain biotin at the 5′ end. This allows binding of the biotinylated amplimers to streptavidin-coated microtiter wells. PCR products are denatured by sodium hydroxide, which allows removal of the non-biotinylated strand Specific digoxigenin(DIG)-labelled oligonucleotide probes are hybridized to the single-stranded immobilized PCR product and hybrids are detected by enzyme-labelled conjugate and colorimetric methods.

For detection of H. pylori DNA, the vacA s-region is used as a target. PCR primers VA1F and biotinylated VA1XR are used for PCR of the vacA s-region. A multiplex PCR can be performed on the vacA s and m-regions. The result of PCR is then tested by the DEIA, using probes aimed at the s-region. In case of a positive result the same PCR mixture, including amplimers from both the vacA s- and m-regions, can subsequently be used on a vacA LiPA

The PCR mixtures can be composed as follows:

| | | |
|---|---|---|
| 1 | μl | target DNA |
| 5 | μl | 10 × PCR buffer (final concentration 10 mM TrisHCl pH 8.3, |
| | | 50 mM KCl, 1-3 mM MgCl₂) |
| 10 | μl | 5 × dNTP's (1 nM) |
| 0.3 | μl | AmpliTaq Gold DNA polymerase (5 units/μl) |
| 1 | μl | VA1F (25 pmoles/μl) |
| 1 | μl | VA1Xr (25 pmoles/μl) |
| 1 | μl | VAMSFb (25 pmoles/μl) |
| 1 | μl | VAMSFc (25 pmoles/μl) |
| 1 | μl | VAMSFd (25 pmoles/μl) |
| 1 | μl | VAMSfe (25 pmoles/μl) |
| 1 | μl | M1R (25 pmoles/μl) |
| 26.3 | μl | water |
| 50 | μl | total |

The following PCR program can be used:

9 min pre-incubation at 94° C.

40 cycles of 1 min 94° C., 1 min 50° C., and 1 min 72° C.

final extension: 5 min at 72° C.

The mixture of probes used for detection of the vacA s-region is shown in table 6.

TABLE 6
Probes for detection of vacA s-region amplimers by
DEIA

| probe | sequence | target |
|---|---|---|
| HpdiaS1 | DIG-CATGCYGCCTTCTTTACAACCGT | s1 |
| HpdiaS2 | DIG-CATGCCGCCTTTTTCACRACCGT | s1 |
| HpdiaS3 | DIG-CATGCCGCTCTTTTTACAACCGT | s1 |
| HpdiaS4 | DIG-CATGCCGCCTTTTTTACAACCGT | s1 |
| HpdiaS5 | DIG-AGTCGCGCYTTTTTYACAACCGT | s2 |

Practically, microtiterplate we %s were precoated with streptavidin. Ten μl of PCR product was mixed with amplimer dilution buffer (1×SSC, 0.1% Tween-20, and 0.004% phenol red). After incubation at 42° C. for 30 minutes, the wells were washed 3 times with 400 μl washing solution (1×SSC, 0.1% Tween-20), The captured PCR products were denatured by addition of 100 μl of 0.1M NaOH into the well and incubated for 5 minutes at room temp. The fluid, containing the unbiotinylated eluted strand was removed. 100 μl hybridization solution containing 1×SSC, 0.1% Tween-20, 0.004% phenol red and 1 pmole of digoxigenin (DIG) labelled oligonucleotide probe(s) were added to the well and incubated for 45 minutes in a waterbath at 42° C. After washing the wells 3 times with washing solution, 100 μl of 75mU/ml anti-digoxigenin-peroxidase conjugate (Boehringer Mannheim) was added and incubated for 15 minutes in a waterbath at 42° C. The unbound conjugate was removed by washing the wells 5 times with washing solution 100 μl of substrate solution containing tetramethylbenzidine (TMB) was added to the wells. After incubation for 15 minutes at room temperature the colour reaction was stopped by addition of 100 μl 0.5M sulphuric acid. The optical density of the wells was read at 450 nm in a microtiter plate reader.

For interpretation of the results, optical densities of the samples were compared with negative controls and borderline positive controls. Table 7 shows the result of a DEIA analysis of 6 samples Sample 1 and 5 yield an optical density that is higher than that of the borderline positive control, these samples are therefore considered positive. The optical density of the other samples is lower than the borderline positive control; they are considered negative.

TABLE 7
Results of a DEIA test

| Sample | OD | conclusion negative |
|---|---|---|
| positive control | 1.178 | |
| borderline pos. control | 0.214 | |
| negative control | 0.102 | |
| sample 1 | >4.0 | positive |
| sample 2 | 0.086 | negative |
| sample 3 | 0.098 | negative |
| sample 4 | 0.108 | negative |
| sample 5 | 2.146 | positive |
| sample 6 | 0.096 | negative |

TABLE 1
Nucleotide sequence of the primers:

| | |
|---|---|
| cag2F | |
| 5′-TTGACCAACAACCACAAACCGAAG-3′ | SEQ ID NO 12 |
| cagR | |
| 5′-CTTCCCTTAATTGCGAGATTCC-3′ | SEQ ID NO 13 |
| VA1-F | |
| 5′-ATGGAAATACAACAAACACAC-3′ | Atherton et al. |
| | 1995 |
| VA1XR | |
| 5′-CCTGARACCGTTCCTACAGC-3′ | SEQ ID NO 14 |
| M1F | |
| 5′-GTGGATGCYCATACRGCTWA-3′ | SEQ ID NO 15 |
| M1R | |
| 5′-RTGAGCTTGTTGATATTGAC-3′ | SEQ ID No 16 |
| HPMGF | |
| 5′-CACAGCCACTTTCAATAACGA-3′ | SEQ ID No 17 |
| HPMGR | |
| 5′-CGTCAAAATAATTCCAAGGG-3′ | SEQ ID No 18 |
| cagSF | |
| 5′-CAACAACCACAAACCGAAG-3′ | SEQ ID No 19 |
| cagSR | |
| 5′-GATTGGTTTTTGATCAGGATC-3′ | SEQ ID No 20 |
| cagFN1 | |
| 5′-GATAAGAAYGATAGGGATAA-3′ | SEQ ID No 21 |
| cagRN1 | |
| 5′-AATACTGATTCTTTTTGG-3 | SEQ ID No 22 |
| VAMSFb | |
| GTGGATGCCCATACGGCTAA | SEQ ID No 23 |
| VAMSFc | |
| GTGGATGCTCATACAGCTWA | SEQ ID No 24 |
| VAMSFd | |
| GTGGATGCCCATACGATCAA | SEQ ID No 25 |
| VAMSFe | |
| GCGAGCGCTCATACGGTCAA | SEQ ID No 26 |

TABLE 2
Nudcotide sequence of the probes:

| | | | |
|---|---|---|---|
| cagApro | SEQ ID NO 1 | GTTGATAACGCTGTCGCTTC | (pos. 94-113) |
| P1S1 | SEQ ID NO 2 | GGAGCRTTRGTCAGCATCAC | (pos. 61-80 of vacA ORF of strain 60190 (Genbank |
| | | | Acc. U05676)) |
| P22S1a | SEQ ID NO 3 | GCTTTAGTAGGAGCRTTRGTC | (pos. 52-72 of vacA ORF of strain 60190 (Genbank |
| | | | Acc. U05676)) |
| P1S1b | SEQ ID NO 4 | GGAGCGTTGATTAGYKCCAT | (pos 61-80) |
| P2S1b | SEQ ID NO 5 | GTTTTAGCAGGAGCGTTGA | (pos 52-72) |
| P1S2(VAS2) | SEQ ID NO 6 | GCTAAYACGCCAAAYGATCC | (pos. 88-107 of vacA ORF of strain Tx30a (Genbank |
| | | | Acc. U29401)) |
| P2S2 | SEQ ID NO 7 | GATCCCATACACAGCGAGAG | (pos. 103-122 of vacA ORF of strain Tx30a (Genbank |
| | | | Acc. U29401)) |
| P1M1 | SEQ ID NO 8 | TTGATACGGGTAATGGTGG | (pos. 1526-1544 of vacA ORF of strain 60190 (Genbank |
| | | | Acc. U05676)) |
| P2M1 | SEQ ID NO 9 | GGGTAATGGTGGTTTCAACA | (pos. 1533-1552 of vacA ORF of strain 60190 (Genbank |
| | | | Acc. U05676)) |
| P1M2 | SEQ ID NO 10 | ACGAATTTAAGAGTGAATGGC | (pos. 1522-1542 of vacA ORF of strain Tx30a (Genbank |
| | | | Acc. U29401)) |
| P2M2 | SEQ ID NO 11 | AGAGCGATAACGGGCTAAACA | (pos. 1577-1597 of vacA ORF of strain Tx30a (Genbank |
| | | | Acc. U29401)) |
| cagprobe3 | SEQ ID NO 27 | GGATTTTTGATCGCTTTATT | (pos. 219-227) |
| P3S1 | SEQ ID NO 28 | GGGYTATTGGTYAGCATCAC | (pos. 26-45) |
| P4S1 | SEQ ID NO 29 | GCTTTAGTRGGGYTATTGGT | (pos. 17-36) |
| P1M1new | SEQ ID NO 30 | TTGATACKGGTAATGGTGG | |
| P2M1new | SEQ ID NO 31 | KGGTAATGGTGGTTTCAACA | |
| P1M2new | SEQ ID NO 32 | KGGTAATGGTGGTTTCAACA | |
| P2M2new | SEQ ID NO 33 | AGAGCGATAAYGGKCTAAACA | |
| P1M3 | SEQ ID NO 34 | AGGGTAGAAATGGTATCGACA | |
| HpdiaS1 | SEQ ID NO 35 | DIG-CATGCYGCCTTCTTTACAACCGT | |
| HpdiaS2 | SEQ ID NO 36 | DIG-CATGCCGCCTTTTTCACRACCGT | |
| HpdiaS3 | SEQ ID NO 37 | DIG-CATGCCGCTCTTTTTACAACCGT | |
| HpdiaS4 | SEQ ID NO 38 | DIG-CATGCCGCCTTTTTTACAACCGT | |
| HpdiaS5 | SEQ ID NO 39 | DTG-AGTCGCGCYTTTTTYACAACCGT | |

### REFERENCES

Akopyanz, N., Bukanov, N. O., Westblom, T. R, Kresovich, S., and Berg, D. E. (1992) Nuc. Acids Res. 20, 5137-5142.

Covacci A., Censini, S., Bugnoli M., Petracca, R., Burroni, D., Macchia, G., Massone, A., Papini, E., Xiang, Z., Figura, N., and Rappuoli R. (1993) Proc. Natl. Acad. Sci. U.S.A 90, 5791-5795.

Cover, T. L., and Blaser, M. J. (1995) Adv. Intern. Med. 41, in press.

Cover, T. L., and Blaser, M. J. (1992) J. Biol. Chen 267, 10570-10575.

Foxall P. A., Hu, L-T., and Mobley, H. L. T. (1992) J. Clin. Microbiol. 30, 739-741.

Hentschel, E., Brandstatter, G., Dragosics, B., Hirschl, A. M., Nemec, H., Schutze, K., Taufer, M., and Wurzer, H. (1993) N. Engl. J. Med. 328, 308-312.

Leunk, R. D., Johnson, P. T., David B. C., Kraft, W. G., and Morgan, D. R. (1988) J. Med. Microbiol. 26, 93-99.

Parsonnet, J., Friedman, G. D., Vandersteen, D. P., Chang, Y., Vogelman, J. H., Orentreich, N., and Sibley, R. K. (1991) N. Engl. J. Med 325, 1127-1131.

Phadnis, S. H. Ilver, D., Janzon, L., Normark, S., and Westblom, T. U. (1994) Infect. Immun. 62, 1557-1565.

Schmitt, W., and Haas, R (1994) Mol. Microbiol. 12, 307-319.

Telford, J. L., Ghiara, P., Dell'Orco, M., Comanducci M., Burrori, D., Bugnoli, M., Tecce, M. F., Cenusi, S., Covacci, A., Xiang, Z., Papini, E., Montecucco, C., Parente, L., and Rappuoli, R. (1994) J. Exp. Med. 179, 1653-1658.

Tummuru, M. K. R., Cover, T. L., and Blaser. M. J. (1993) Infect. Immun. 61,11799-1809.

What is claimed is:

1. A method for the detection of Helicobacter pylori (H. pylori) present in a sample comprising the steps of:
amplifying the polynucleic acids of the m and s regions of the vacA gene with a pair of primers, wherein one of said primers is selected from the group consisting of: SEQ ID NOS: 14-18 and, another of said primers is selected from the group consisting of SEQ ID NOS: 23-26 and 277;
hybridizing the polynucleic acids obtained with at least one probe hybridizing to a conserved region of the vacA gene and at least one probe hybridizing to a variable region of the vacA gene, thus forming hybrids;
detecting the hybrids formed; and
determining the presence or absence of H. pylori in a sample from the hybridization signals obtained.

2. A method according to claim 1 wherein said primer pair for the amplification step comprises VA1F (SEQ ID NO:277) and VA1XR (SEQ ID NO:14).

3. A method according to claim 1, wherein the hybridization step comprises hybridizing the polynucleic acids obtained in the amplification step with a set of probes, under hybridization and wash conditions, comprises at least one probe hybridizing to a conserved region of the vacA gene of H. pylori, and at least one vacA-derived probe selected from the group consisting of SEQ ID NOS:35-39.

4. A probe for use in a method of detecting the presence of H. pylori, said probe comprising a sequence selected from the group consisting of SEQ ID NOS:35-39.

5. The method of claim 1 additionally comprising the step of releasing, isolating, or concentrating the H. pylori polynucleic acids in the original sample.

6. The method according to claim 1, wherein the hybridization step is a reverse hybridization step, wherein the probes are immobilized on a solid support.

7. The method according to claim 6, wherein said probes are immobilized as parallel lines on a solid support.

8. The method according to claim 6, wherein said solid support is a membrane strip.

9. A kit for detecting and/or typing H. pylori strains in a sample liable to contain it, comprising the following components:
at least one probe selected from the group consisting of: SEQ ID NOS:1-11 and 27-39 or variants thereof, with said probe and/or other probes applied;
a buffer or components necessary to produce the buffer enabling an amplification or a hybridization reaction between said probes and the amplified products; and
a means for detecting the hybrids resulting from said hybridization.

10. The method according to claim 9, wherein said solid support is a microtiter plate.

11. The method of claim 1, further comprising amplifying the polynucleic acids of the cagA gene of H. pylori with a primer pair that amplifies a conserved region of the cagA gene of all H.pylori strains.

12. The method of claim 5, wherein each primer from said primer pair comprises a primer selected from the group consisting of: SEQ ID NOS: 1, 12-13, 19-22, and 27.

13. A method according to claim 1 for the detection and/or typing of alleles of the cagA and vacA gene of H.pylori present in a sample using a set of probes and/or primers specially designed to detect and/or to amplify and/or to type the said alleles, with said probes selected from the group consisting of: SEQ ID NOS: 1-11 and 27-39 and primers being selected from the group consisting of: SEQ ID NOS: 12-26 and variants thereof that can amplify said vacA or cagA region of all strains of H.pylori.

14. An isolated vacA polynucleotide sequence selected from the group consisting of: SEQ ID NOS: 40-91 and SEQ ID NOS: 115-276.

15. A method for the detection and/or typing of Helicobacter pylori (H.pylori) strains present in a sample comprising the steps of:
amplifying the polynucleic acids of the m and s regions of the vacA gene and a conserved region of the cagA gene, with a pair of primers, wherein said vacA primers are selected from the group consisting of: SEQ ID NOS: 14-18, 23-26, and 277;
hybridizing the polynucleic acids obtained with at least one probe hybridizing to a conserved region of the cagA gene and at least one probe hybridizing to a variable region of the vacA gene, thus forming hybrids;
detecting the hybrids formed;
detecting and/or typing H.pylori strains present in a sample from the differential hybridization signals obtained; wherein said typing comprises the allele-specific detection of a strain according to the vacA polynucleic acid alleles.

16. The method of claim 11, wherein said cagA primers are selected from the group consisting of SEQ ID NOS:12-13, and 19-22.

17. The method of claim 1, wherein said probes has compatible hybridization and wash conditions.

18. The method of claim 11 wherein the hybridization step is a reverse hybridization step, wherein said probes are immobilized on a solid support.

19. The method according to claim 11 wherein the polynucleic acids obtained in the amplification step are immobilized on a solid support and the subsequent hybridization step is carried out on said solid support.

20. A probe for use in a method according to claim 11, wherein said vacA probe is selected from the group consisting of: SEQ ID NOS: 2-11 and 28-34.

21. An oligonucleotide primer, wherein said vacA primer is selected from the group consisting of SEQ ID NOS: 14-18, and 23-26.

22. A probe for detection or typing of vacA, said probe selected from the group consisting of SEQ ID NOS:2-11, and 28-39.

23. A vacA-specific oligonucleotide selected from the group consisting of SEQ ID NOS: 14-18 and 23-26.

24. A method according to claim 11 wherein at least one of said probes is selected from the group consisting of SEQ ID NOS:2-11 and 28-39 and wherein said primers are selected from the group consisting of SEQ ID NOS:14-18 and 23-26.
